(19) United States
(12) Patent Application Publication
(10) Pub. No.: US 2008/0262021 A1
(43) Pub. Date: Oct. 23, 2008

(54) 1H-Imidazoquinoline Derivatives as Prote In Kinase Inhibitors

(75) Inventors: Hans-Georg Capraro, Rheinfelden (DE); Giorgio Caravatti, Bottmingen (CH); Pascal Furet, Thann (FR); Carlos Garcia-Echeverria, Basel (CH); Patricia Imbach, Kaiseraugst (CH); Frederic Stauffer, Reinach (CH)

Correspondence Address: NOVARTIS;CORPORATE INTELLECTUAL PROPERTY, ONE HEALTH PLAZA 104/3, EAST HANOVER, NJ, 07936-1080, US

(73) Assignee: NOVARTIS AG, Basel (CH)

(21) Appl. No.: 10/579,587

(22) PCT Filed: Nov. 19, 2004
(86) PCT No.: PCT/EP2004/013178

§ 371 Date: Jul. 14, 2008

Related U.S. Application Data
(60) Provisional application No. 60/524,229, filed on Nov. 21, 2003.

(51) Int. Cl. A61K 31/4745; C07D 471/04; A61P 35/04

(52) U.S. Cl. 514/293; 546/82

(57) ABSTRACT

The invention relates to imidazoquinolines of formula (I) for use in the treatment of protein kinase dependent diseases; pharmaceutical preparations comprising an imidazoquinoline, especially for the treatment of a protein kinase dependent disease; novel imidazoquinolines; and a process for the preparation of the novel imidazoquinolines.

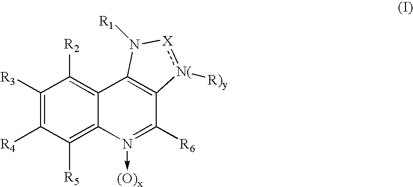

### BACKGROUND OF THE INVENTION

The invention relates to the use of imidazoquinolines and salts thereof in the treatment of protein kinase dependent diseases and for the manufacture of pharmaceutical preparations for the treatment of said diseases; imidazoquinolines for use in the treatment of protein kinase dependent diseases; a method of treatment against said diseases, comprising administering the imidazoquinolines to a warm-blooded animal, especially a human; pharmaceutical preparations comprising an imidazoquinoline, especially for the treatment of a protein kinase dependent disease; novel imidazoquinolines; and a process for the preparation of the novel imidazoquinolines.

### SUMMARY OF THE INVENTION

Recently, the concept of treating proliferative diseases by using drugs designed specifically against abnormally active protein kinases has been definitely proven in the treatment of chronic myeloic leukemia (CML) where a first product has now been approved for successful treatment. Clinical studies showed that the drug (N-{5-[4-(4-methyl-piperazino-methyl)-benzoylamido]-2-methylphenyl}-4-(3-pyridyl)-2-pyrimidine-amine, especially in the form of the methane sulfonate (monomesylate) salt called STI571, which is sold, e.g., under the tradename Gleevec®, has impressive activity against chronic phase CML. Typical for CML is a characteristic t(9; 22) translocation that juxtaposes the 5′ end of the bcr gene with the 3′ end of the abl gene, resulting in a unique 210 kDa fusion protein p210^{bcr/abl} with constitutive activity. The result is a p210^{bcr/abl}-induced transformation ultimately leading to CML. STI571 is a reversible inhibitor that occupies the ATP binding pocket of p210^{bcr/abl} and stabilizes the kinase in an inactive conformation. This inhibitory action appears to be the basis for its action against CML.

Over-expression or constitutive-expression (activity) of protein kinases appears to be a general principle for transformations that finally lead to proliferative growth of cells and thus cancer, psoriasis or other proliferative diseases. Protein kinase B (PKB), also known as Akt, is a member of a conserved family of kinases that includes PKBα, PKBβ and PKBγ in humans. This serine/threonine kinase mediates the physiological effects of several peptide growth factors, including platelet-derived growth factor, insulin and insulin-like growth factor-I. PKB contains a pleckstrin homology (PH) domain in its amino-terminal domain, a kinase domain in the middle and a regulatory domain in the carboxy-terminal region. The binding of phosphoinositides to the PH domain of PKB recruits PKB to the plasma membrane where it is phosphorylated on threonine-308 (Thr308) and on serine-473 (Ser473). Activation of the PKB pathways results in cellular proliferative, as well as antiapoptotic tumor cell responses. PKBα is amplified in 20% of gastric adenocarcinoma and PKBα is amplified in 15% of ovarian cancers, 12% of pancreatic cancers and 3% of breast carcinomas. PKBα expression and activity is elevated in estrogen receptor negative breast cancer cells and in androgen-independent prostate cancer.

The tumor suppressor protein PTEN is a lipid phosphatase that converts PIP3 into PIP2, and therefore down-regulates all pathways that are PIP3 dependent See Cantley et al., Proc Nat. Acad Sci USA, Vol. 96, pp. 4240-4245 (1999); Vazquez et al., Biochimica et Biophysica Acta, Vol. 1470, pp. M21-M35 (2000); and Simpson et al., Exper Cell Res, Vol. 264, pp. 29-41 (2001). The PTEN tumor suppressor gene is frequently found mutated or deleted in gliobastoma [see Li et al., Science, Vol. 275, pp. 1943-1947 (1997); and Steck et al., Nat Genetics, Vol. 15, pp. 356-362 (1997)], endometrial carcinoma [see Nagase et al., Br J Cancer; Vol. 74, pp. 1979-1983 (1996)]; Peiffer et al., Cancer Res, Vol. 55, pp. 1922-1926 (1995), prostate adenocarcinoma [see Gray et al., Cancer Res, Vol. 55, No. 21, pp. 4800-4803 (1995); and Ittmann, Cancer Res, Vol. 56, pp. 2143-2147 (1996)], breast adenocarcinoma [see Perren et al., Am J Pathol, Vol. 155, No. 4, pp. 1253-1260 (1999)] and melanoma. See Robertson et al., Cancer Res, Vol. 59, No. 15, pp. 3596-3601 (1999). PTEN mutations have also been found in cancers of the bladder, lung and lymphatic systems. See Cairns et al., Oncogene, Vol. 16, pp. 3215-3218 (1998); Gronbaek et al., Blood, Vol. 91, pp. 4388-4390 (1998); and Kim et al., Oncogene, Vol. 16, pp. 89-93 (1998). Tumor cell lines with an inactive PTEN allele exhibit elevated PKB kinase activity. See Haas-Kogan et al., Curr Biol, Vol. 8, pp. 1195-1198 (1998); Whang et al., Proc Natl Acad Sci USA, Vol. 95, pp. 5246-5250 (1998); and Wu et al., Proc Natl Acad Sci USA, Vol. 95, pp. 15587-15591 (1998). Consequently, mutations in PTEN that inactivate its lipid phosphatase function allow unregulated PKB activity, a major player in the PI-3K pathway, leading to uncontrolled cell proliferation and increased survival.

Germline mutations of PTEN are also associated to Cowden syndrome, Lhermitte-Dudos disease and Bannayan-Zonana syndrome. See Maehama and Dixon, Trends Cell Biol, Vol. 9, pp. 125-128 (1990). Therefore, compounds that down-regulate the kinase activity of PKB may prove to be of clinical interest for single and combined anticancer treatment modalities.

S6K1, also known as p70 S6 kinase is a serine/threonine kinase that phosphorylates the ribosomal protein S6 (rpS6) in response to mitogen stimulation. Phosphorylation of rpS6 leads of increased expression of the mRNA family members characterized by an oligopyrimidine tract at their 5′ transcriptional start sites (5′TOP) and results in upregulation of protein synthesis because the 5′TOP family mRNAs encode components of the translational apparatus including ribosomal proteins and translational elongation factors. S6K1-deficient mice are protected against diet-induced weight gain and insulin resistance due to the increase in energy expenditure and fatty acid metabolism, suggesting that S6K1 is a potential target of metabolic diseases, e.g., type II diabetes, obesity, hyperlipidemia and atherosclerosis.

The compounds of the present invention also exhibit powerful inhibition of the tyrosine kinase activity of anaplastic lymphoma kinase (ALK) and the fusion protein of NPM-ALK. This protein tyrosine kinase results from a gene fusion of nucleophosmin (NPM) and the anaplastic lymphoma kinase (ALK), rendering the protein tyrosine kinase activity of ALK ligand-independent. NPM-ALK plays a key role in signal transmission in a number of hematopoetic and other human cells leading to hematological and neoplastic diseases, for example in anaplastic large-cell lymphoma (ALCL) and non-Hodgkin's lymphomas (NHL), specifically in ALK+NHL or Alkomas, in inflammatory myofibroblastic tumors (IMT) and neuroblastomas. See Duyster et al., Oncogene, Vol. 20, pp. 5623-5637 (2001). In addition to NPM-ALK, other gene fusions have been identified in human hematological and neoplastic diseases; mainly TPM3-ALK (a fusion of nonmuscle tropomyosin with ALK).

RET encodes a transmembrane receptor of the protein tyrosine kinase family. RET is the receptor of growth factors belonging to the glial cell line-derived neurotrophic factor (GDNF) family. This family is comprised of the GDNF, neurturin (NTN), persephin (PSP), and artemin, which all have trophic influences on a variety of neuronal populations. These ligands interact with multimeric receptors composed of high-affinity glycosyl-phosphatidylinositol (GPI)-linked receptors and the RET kinase. Ligand-dependent RET activation can promote neuronal cell survival and differentiation. Insufficient PTK signaling may be responsible for developmental diseases. Gain of function of the RET receptor PTK is associated with human cancer.

What is desirable from the point of view of possible treatments of proliferative diseases is to have a plethora of compound classes each tailored to specific protein kinases or protein kinase classes, thus allowing to come to specific treatments. Therefore, a strong need exists to find new classes of compounds allowing for such specific inhibitory effects.

### SUMMARY OF THE INVENTION

The class of imidazoquinoline compounds described herein, especially novel compounds falling under this class, has surprisingly been found to have pharmaceutically advantageous properties, inter alia, allowing for the inhibition of specific types or classes or groups of protein kinases, especially PKB, ALK, RET, S6K1 or any combinations of two or more of these. The class of imidazoquinoline compounds described herein further inhibit mutants of said kinases. In addition to this established activity, the imidazoquinolines have the advantage that their backbone in addition allows for a plethora of substitution patterns that offer a broad possibility to achieve a fine tuning for specific interaction with the ATP-binding site of the kinases, thus opening a new perspective and providing kinase inhibitors of various degrees of specificity.

### DETAILED DESCRIPTION OF THE INVENTION

The invention in particular relates to imidazoquinolines, especially compounds of the formula (I)

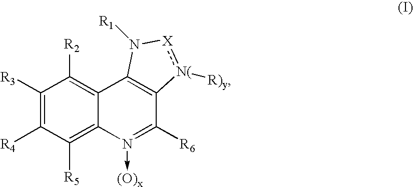

wherein
each of x and y is, independently of the other, 0 or 1;
R₁ is an organic moiety that can be bound to nitrogen;
X is C═O or C═S with the proviso that then the dashed line bonding X to N is absent, so that X is bound to the adjacent N via a single bond, and with the proviso that then y is 1 and R is hydrogen or an organic moiety that can be bound to nitrogen;
or X is (CR₇), wherein R₇ is hydrogen or an organic or inorganic moiety, with the proviso that then the dashed line bonding X to N is a bond, so that X is bound to the adjacent N via a double bond, and with the proviso that then y is zero or y is 1 and then —R is →O; and
each of R₂, R₃, R₄, R₅ and R₆, independently of the others, is an organic moiety or hydrogen or an inorganic moiety, with the proviso that R₃ cannot be unsubstituted phenyl unless R₁ is phenyl substituted with an heterocyclic ring;
   or a pharmaceutically acceptable salts thereof and use of compounds of formula (I) in the treatment of protein kinase dependent diseases or for the manufacture of pharmaceutical preparations for the treatment of protein kinase dependent diseases.

The present invention is also directed to methods of treating protein kinase dependent diseases comprising administering imidazoquinoline compounds of the formula (I) to a warm-blooded animal, especially a human; pharmaceutical preparations comprising an imidazoquinoline compound of the formula (I), especially for the treatment of a protein kinase dependent disease; novel imidazoquinoline compounds of the formula (I); a process for the manufacture of the novel imidazoquinoline compounds of the formula (I); the manufacture of a pharmaceutical preparation for the treatment of protein kinase diseases, and novel starting materials and intermediates for their manufacture.

The general terms used hereinbefore and hereinafter preferably have within the context of this disclosure the following meanings, unless otherwise indicated:

The prefix “lower” denotes a radical having 1 carbon atom up to and including a maximum of 7 carbon atoms, especially 1 carbon atom up to and including a maximum of 4 carbon atoms, the radicals in question being either linear or branched with single- or multiple-branching. Lower alkyl, e.g., is methyl, ethyl, n-propyl, sec-propyl, n-butyl, isobutyl, sec-butyl, tert-butyl, n-pentyl, n-hexyl or n-heptyl.

An organic moiety that can be bound to nitrogen is preferably unsubstituted or substituted alkyl, unsubstituted or substituted alkenyl, unsubstituted or substituted alkynyl, unsubstituted or substituted aryl, unsubstituted or substituted aryl-lower alkyl or aryl-lower alkoxy, unsubstituted or substituted heterocyclyl, unsubstituted or substituted heterocyclyl lower alkyl or lower alkoxy, unsubstituted or substituted cycloalkyl or unsubstituted or substituted cycloalkenyl.

An organic moiety is preferably unsubstituted or substituted alkyl, unsubstituted or substituted alkenyl, unsubstituted or substituted alkynyl, unsubstituted or substituted unsubstituted or substituted aryl, unsubstituted or substituted heterocyclyl, unsubstituted or substituted cycloalkyl or unsubstituted or substituted cycloalkenyl, unsubstituted or substituted arylcarbonylamino, amino substituted by one or two moieties selected from the group consisting of lower alkyl, substituted lower alkyl moieties, aryl, cycloalkyl and mercapto-lower alkyl, alkyloxy or cyano.

Halo or halogen is preferably fluoro, chloro, bromo or iodo, most preferably fluoro, chloro or bromo.

Alkyl preferably has up to 20 carbon atoms, more preferably up to 12 carbon atoms and is linear or branched one or more times; preferred is lower alkyl, especially C₁-C₄alkyl. Alkyl is linear or cyclic and unsubstituted or substituted, preferably by one or more substituents independently selected from those mentioned below under “substituted”. Unsubstituted alkyl, preferably lower alkyl, or hydroxyalkyl, especially hydroxy-lower alky, e.g., 2-hydroxyethyl; or cycloalkyl, e.g., cyclopropyl.

Among the moieties corresponding to substituted alkyl, unsubstituted or substituted aryl-lower alkyl (especially preferred), heterocyclyl-lower alkyl or cycloalkyl-lower alkyl are also preferred.

Aryl-lower alkyl is preferably lower alkyl that is substituted (preferably terminally or in 1-position) by unsubstituted or substituted aryl as defined below, especially phenyl-lower alkyl, such as benzyl or phenylethyl, especially 1-phenylethyl.

Heterocyclyl-lower alkyl is preferably lower alkyl that is substituted (preferably terminally) by unsubstituted or substituted haterocyclyl as defined below.

Cycloalkyl-lower alkyl is preferably lower alkyl that is substituted (preferably terminally) by unsubstituted or substituted cycloalkyl as defined below.

Alkenyl is preferably a moiety with one or more double bonds and preferably has 2-20 carbon atoms, more preferably up to 12 carbon atoms; it is linear or branched one or more times (as far as possible in view of the number of carbon atoms). Preferred is C₂-C₇alkenyl, especially C₃-C₄alkenyl, such as allyl or crotyl. Alkenyl can be unsubstituted or substituted, especially by one or more, more especially up to three of the substituents mentioned below under “substituted”. Substituents, such as amino or hydroxy (with free dissociable hydrogen) preferably are not bound to carbon atoms that participate at a double bond, and also other substituents that are not sufficiently stable are preferably excluded. Unsubstituted alkenyl, in particular, C₂-C₇alkenyl is preferred.

Alkynyl is preferably a moiety with one or more triple bonds and preferably has 2-20 carbon atoms, more preferably up to 12 carbon atoms; it is linear of branched one or more times (as far as possible in view of the number of carbon atoms). Preferred is C₂-C₇alkynyl, especially C₃-C₄alkynyl, such as ethynyl or propyn-2-yl. Alkynyl can be unsubstituted or substituted, especially by one or more, more especially up to three of the substituents mentioned below under “substituted”. Substituents, such as amino or hydroxy (with free dissociable hydrogen) preferably are not bound to carbon atoms that participate at a triple bond, and also other substituents that are not sufficiently stable are preferably excluded. Unsubstituted alkynyl, in particular, C₂-C₇alkynyl is preferred.

Aryl, preferably, has a ring system of not more than 20 carbon atoms, especially not more than 16 carbon atoms, is preferably mono-, bi- or tric-cyclic, and is unsubstituted or substituted preferably as defined below under “substituted”. For example, aryl is selected from phenyl, naphthyl, indenyl, azulenyl and anthryl, and is preferably in each case unsubstituted or halo, especially fluoro, chloro, bromo or iodo; halo-lower alkyl, especially trifluoromethyl; hydroxyl; amino, mono or disubstituted amino; cyclic amino; amino-lower alkyl, e.g., aminomethyl, 2-aminoethyl or 3-aminopropyl, which when bonded to the ring nitrogen, is connected by a bridge consisting of O, S or N; lower alkoxy, e.g., methoxy; hydroxy-lower alkyl, e.g., hydroxymethyl or 2-hydroxyethyl; lower alkyl, e.g., methyl or ethyl; cyano; cyano-lower alkyl, e.g., 2-cyanoethyl and 3-cyanopropyl; amidino; N-hydroxyamidino; amidino-lower alkyl, e.g., 2-amidino-ethyl; or N-hydroxyamidino-lower alkyl, e.g., 2-(N-hydroxyamidino)ethyl substituted phenyl or (especially 1- or 2-)naphthyl. The aryl group may also be substituted with a heterocycle, heterocyclyl lower alkyl, heteroaryl or heteroaryl lower alkyl as defined hereinbelow. Unsubstituted or substituted aryl, preferably phenyl; hydroxyphenyl, e.g., 4-hydroxyphenyl; or methoxyphenyl, e.g., 2-, 3- or 4-methoxyphenyl; benzo[1,3]dioxolo or lower alkyl, e.g., methyl or ethyl is especially preferred as organic moiety that can be bound to nitrogen or as organic moiety R₂ to R₇.

In arylcarbonylamino, aryl is preferably aryl as defined in the last paragraph, especially benzoylamino.

Heterocyclyl is preferably a heterocyclic radical that is unsaturated, saturated or partially saturated in the bonding ring and is preferably a monocyclic or in a broader aspect of the invention bicyclic or tricyclic ring; has 3-24 ring atoms, more preferably 4-16 ring atoms, wherein at least in the ring bonding to the radical of the molecule of formula (I) one or more, preferably 1-4 carbon ring atoms, especially one or two carbon ring atoms are replaced by a heteroatom selected from the group consisting of nitrogen, oxygen and sulfur; the bonding ring preferably having 4-12 ring atoms, especially 4-7 ring atoms; heteroaryl being unsubstituted or substituted by one or more, especially 1-4 substituents independently selected from the group consisting of the substituents defined below under “substituted”; especially being a heteroaryl radical selected from the group consisting of oxiranyl, azirinyl, 1,2-oxathiolanyl, imidazolyl, thienyl, furyl, tetrahydrofuryl, indolyl, azetidinyl, pyranyl, thiopyranyl, thianthrenyl, isobenzofuranyl, benzofuranyl, chromenyl, 2H-pyrrolyl, pyrrolyl, pyrrolinyl, pyrrolidinyl, imidazolyl, imidazolidinyl, benzimidazolyl, pyrazolyl, pyrazinyl, pyrazolidinyl, pyranyol, thiazolyl, isothiazolyl, dithiazolyl, oxazolyl, isoxazolyl, pyridyl, pyrazinyl, pyrimidinyl, piperidyl, piperazinyl, pyridazinyl, morpholinyl, thiomorpholinyl, indolizinyl, isoindolyl, 3H-indolyl, indolyl, benzimidazolyl, cumaryl, indazolyl, triazolyl, tetrazolyl, purinyl, 4H-quinolizinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, quinoxalyl, quinazolinyl, quinazolinyl, cinnolinyl, pteridinyl, carbazolyl, β-carbolinyl, phenanthridinyl, acridinyl, perimidinyl, phenanthrolinyl, furazanyl, phenazinyl, phenothiazinyl, phenoxazinyl, chromenyl, isochromanyl and chromanyl; each of these radicals being unsubstituted or substituted by one to two radicals selected from the group consisting of lower alkyl, especially methyl or tert-butyl; lower alkoxy, especially methoxy; and halo, especially fluoro or chloro. Unsubstituted or substituted heterocyclyl or lower alkyl heterocyclyl, e.g., benzo[1,3]dioxlo, indolyl, benzofuranyl, thienyl, pyridyl, imidazolinyl, lower alkyl imidazolinyl, morpholinyl, piperazinyl, lower alkyl piperazinyl, piperidino, piperidyl, pyrrolidinyl and azetidinyl, are preferred.

Cycloalkyl is preferably C₃-C₁₀cycloalkyl, especially cyclopropyl, dimethylcyclopropyl, cyclobutyl, cyclopentyl, cyclohexyl or cycloheptyl; cycloalkyl being unsubstituted or substituted by one or more substituents, especially 1-3 substituents independently selected from the group consisting of the substituents defined below under “substituted”.

Cycloalkenyl is preferably C₅-C₁₀cycloalkenyl, especially cyclopentenyl, cyclohexenyl or cycloheptenyl; cycloalkenyl being unsubstituted or substituted by one or more substituents, especially 1-3 substituents, independently selected from the group consisting of the substituents defined below under “substituted”.

An inorganic moiety R₂ to R₇ is, preferably halogen, especially fluoro, chloro, bromo or iodo; hydroxy; amino; or nitro.

An organic moiety R₂ to R₇ is selected from the organic moieties, mentioned above, for organic moieties that can be bound to nitrogen (for R₁) or is alternatively selected from the group consisting of unsubstituted or substituted alkyl, e.g., lower alkyl; unsubstituted or substituted alkoxy, e.g., lower alkoxy; or phenyl-lower alkoxy, e.g., methoxy; or lower alkanoyloxy, e.g., acetoxy; amino substituted by one or two moieties selected from the group consisting of lower alkyl, e.g., methyl or n-butyl; hydroxy-lower alkyl, e.g., 2-hydroxyethyl; mercapto-lower alkyl, e.g., 2-mercaptoethyl; unsubstituted or substituted C₅-C₁₄aryl as defined above, especially phenyl; a heteroaryl being unsubstituted or substituted by one or more, especially 1-3, substituents independently selected from the group consisting of the substituents defined below under “substituted”; especially being pyridyl (or an N-oxide of pyridyl) which is unsubstituted or substituted by one to two radicals selected from the group consisting of lower alkyl, e.g., methyl; lower alkoxy, e.g., methoxy; halo, e.g., fluoro; or —NR₈R₉, wherein R₈ and R₉ can be the same or different and are independently H; lower alkyl, e.g., methyl, ethyl or propyl; lower cycloalkyl, e.g., cyclopropyl; or the R₈ and R₉ can, with the N atom, form a 3- to 8-membered heterocyclic ring containing 1-4 nitrogen, oxygen or sulfur atoms, e.g., azetidinyl, pyrrolidinyl, piperidino, morpholinyl, imidazolinyl, piperazinyl or lower alkyl-piperazinyl; cycloalkyl as defined above, especially C₃-C₆cycloalkyl; lower alkanoyl, preferably as single amino substituent or in combination with another of the non-acyl moiety just mentioned; and benzoyl or phenyl-lower alkanoyl, preferably as single amino substituent or in combination with another of the non-acyl moiety just mentioned; amino; amino-lower alkyl; cyano; cyano-lower alkyl, e.g., cyanomethyl; amidino; N-hydroxyamidino; amidino-lower alkyl, e.g., -methyl; or N-hydroxyamidino-lower alkyl, e.g., -methyl.

Preferably, only up to five, more preferably up to three of R₂, R₃, R₄, R₅, R₆ and R₇ are/is other than hydrogen, i.e., an inorganic or organic moiety.

A very preferred group of compounds of formula (I) are those wherein R₃ is one of the organic moieties other than hydrogen, especially those mentioned as being preferred above.

“Substituted”, wherever used for a moiety, means that one or more hydrogen atoms in the respective moiety, especially up to 5 hydrogen atoms, more especially up to three of the hydrogen atoms are replaced independently of each other by the corresponding number of substituents, which preferably are independently selected from the group consisting of lower alkyl, e.g., methyl, ethyl or propyl; halo, e.g., F, Cl, Br or I; halo-lower alkyl, e.g., trifluoromethyl; hydroxy; carboxy; lower alkoxy, e.g., methoxy; phenyl-lower alkoxy; lower alkanoyloxy; lower alkanoyl; hydroxy-lower alkyl, e.g., hydroxymethyl or 2-hydroxyethyl; amino; mono- or di-substituted amino; cyclic amino; amino-lower alkyl, e.g., aminomethyl, 2-aminoethyl or 3-aminopropyl; N-lower alkylamino; N,N-di-lower alkylamino; N-phenyl-lower alkylamino; N,N-bis(phenyl-lower alkyl)-amino; amino lower alkoxy; lower alkanoylamino; benzoylamino; carbamoyl-lower alkoxy; N-lower alkylcarbamoyl-lower alkoxy or N,N-di-lower alkylcarbamoyl-lower alkoxy; amidino; N-hydroxy-amidino; guanidino; amino-lower alkyl, e.g., aminomethyl or 2-aminoethyl; amidino-lower alkyl, e.g., 2-amidinoethyl; N-hydroxyamidino-lower alkyl, e.g., N-hydroxy-amidino-methyl or -2-ethyl; carboxy; lower alkoxycarbonyl; phenyl-, naphthyl- or fluorenyl-lower alkoxycarbonyl, e.g., benzyloxycarbonyl; lower alkanoyl; sulfo; lower alkanesulfonyl, e.g., methanesulfonyl (CH₃—S(O)₂—); sulfonamide (NH₂—S(O)₂—); dioxolo; phosphono (—P(═O)(OH)₂); hydroxy-lower alkoxy phosphoryl or di-lower alkoxyphosphoryl; carbamoyl; mono- or di-lower alkylcarbamoyl; sulfamoyl; sulfamide; mono- or di-lower alkylaminosulfonyl; cyano-lower alkyl, e.g., cyanomethyl; and cyano; C₅-C₁₆aryl, e.g., phenyl or naphthyl, where C₅-C₁₆aryl is substituted with any of the substituents defined above, and especially is phenyl which is unsubstituted or substituted with up to 4 substituents, preferably up to 2 substituents, wherein the substituents are the same or different and are independently selected from halo, e.g., Cl or F; cyano; cyano lower alkyl, e.g., cyanomethyl, cyanoethyl and cyanopropyl; lower alkyl; lower alkoxy; amino-lower alkyl; amino-lower alkoxy; amino-lower alkyl sulfanyl; or thiol-lower alkyl, wherein the amino group can be mono- or di-substituted, e.g., —(C₁-C₇)ₘNR₈R₉; or —O—(C₁-C₇)ₘNR₈R₉,

wherein
m is 0 or 1; and
R₈ and R₉ can be the same or different and are independently H; lower alkyl, e.g., methyl, ethyl or propyl; lower cycloalkyl, e.g., cyclopropyl, or
R₈ and R₉, together with the N atom, form a 3- to 8-membered heterocyclic ring containing 1-4 nitrogen, oxygen or sulfur atoms, e.g., azetidinyl, pyrrolidinyl, piperidino, morpholinyl, imidazolinyl, piperazinyl or lower alkyl-piperazinyl.

“Substituted” also includes amino-carbonyl-lower alkyl, e.g., R₈R₉—N—C(O)—CH₂—, wherein R₈ and R₉ are as defined above; heterocyclyl; heterocyclyl-lower alkyl; heterocyclyl-lower alkoxy or heterocyclyl-lower alkanesulfanyl, wherein the heterocyclyl is a substituted or unsubstituted 3- to 8-membered heterocyclic ring containing 1-4 nitrogen, oxygen or sulfur atoms, e.g., imidazolyl, imidazolinyl, pyrrolidinyl, morpholinyl, azetidinyl, pyridyl, piperidino, piperidyl, piperazinyl or lower alkyl-piperazinyl; C₃-C₁₀cycloalkyl, e.g., cyclopropyl or cyclohexyl; hydroxyC₃-C₈cycloalkyl, e.g., hydroxy-cyclohexyl; heteroaryl with 4 or 6 ring atoms and 1-4 ring heteroatoms selected from O, N and S, especially furyl; 1,4 oxazinyl; or pyridyl; or —NR₈R₉, wherein R₈ and R₉ can be the same or different and are independently H; lower alkyl, e.g., methyl, ethyl or propyl; lower cycloalkyl, e.g., cyclopropyl; or the R₈ and R₉ can, with the N atom, form a 3- to 8-membered heterocyclic ring containing 1-4 nitrogen, oxygen or sulfur atoms, e.g., azetidinyl, pyrrolidinyl, piperidino, morpholinyl, imidazolinyl, piperazinyl or lower alkyl-piperazinyl. It goes without saying that substituents are only at positions where they are chemically possible, the person skilled in the art being able to decide (either experimentally or theoretically) without inappropriate effort which substitutions are possible and which are not. For example, amino or hydroxy groups with free hydrogen may be unstable if bound to carbon atoms with unsaturated, e.g., olefinic, bonds.

Salts are preferably the pharmaceutically acceptable salts of compounds of formula (I) if they are carrying salt-forming groups.

Salt-forming groups in a compound of formula (I) are groups or radicals having basic or acidic properties. Compounds having at least one basic group or at least one basic radical, e.g., amino; a secondary amino group not forming a peptide bond or a pyridyl radical, may form acid addition salts, e.g., with inorganic acids, such as hydrochloric acid, sulfuric acid or a phosphoric acid; or with suitable organic carboxylic or sulfonic acids, e.g., aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid; or amino acids, such as arginine or lysine; aromatic carboxylic acids, such as benzoic acid; 2-phenoxy-benzoic acid; 2-acetoxy-benzoic acid; salicylic acid; 4-aminosalicylic acid; aromatic-aliphatic carboxylic acids, such as mandelic acid or cinnamic acid; heteroaromatic carboxylic acids, such as nicotinic acid or isonicotinic acid; aliphatic sulfonic acids, such as methane-, ethane- or 2-hydroxyethanesulfonic acid; or aromatic sulfonic acids, e.g., benzene-, p-toluene- or naphthalene-2-sulfonic acid. When several basic groups are present mono- or poly-acid addition salts may be formed.

Compounds of formula (I) having acidic groups, a carboxy group or a phenolic hydroxy group, may form metal or ammonium salts, such as alkali metal or alkaline earth metal salts, e.g., sodium, potassium, magnesium or calcium salts; or ammonium salts with ammonia or suitable organic amines, such as tertiary monoamines, e.g., triethylamine or tri(2-hydroxyethyl)-amine, or heterocyclic bases, e.g., N-ethyl-piperidine or N,N′-dimethylpiperazine. Mixtures of salts are possible.

Compounds of formula (I) having both acidic and basic groups can form internal salts.

For the purposes of isolation or purification, as well as in the case of compounds that are used further as intermediates, it is also possible to use pharmaceutically-unacceptable salts, e.g., the picrates. Only pharmaceutically-acceptable, non-toxic salts may be used for therapeutic purposes, however, and those salts are therefore preferred.

Owing to the close relationship between the novel compounds in free form and in the form of their salts, including those salts that can be used as intermediates, e.g., in the purification of the novel compounds or for the identification thereof, any reference hereinbefore and hereinafter to the free compounds shall be understood as including the corresponding salts, where appropriate and expedient.

Where the plural form is used for compounds, salts, pharmaceutical preparations, diseases and the like, this is intended to mean also a single compound, salt or the like. Any asymmetric carbon atom may be present in the (R)-, (S)- or (R,S)-configuration, preferably in the (R)- or (S)-configuration. Substituents at a double bond or a ring may be present in cis-(=Z-) or trans (=E-) form. The compounds may thus be present as mixtures of isomers or preferably as pure isomers, preferably as enantiomer-pure diastereomers or pure enantiomers.

The present invention also relates to pro-drugs of a compound of formula (I) that convert in vivo to the compound of formula (I) as such. Any reference to a compound of formula (I) is therefore to be understood as referring also to the corresponding pro-drugs of the compound of formula (I), as appropriate and expedient.

The terms “treatment” or “therapy” refer to the prophylactic or preferably therapeutic including, but not limited to, palliative, curing, symptom-alleviating, symptom-reducing, kinase-regulating and/or kinase-inhibiting, treatment of said diseases, especially of the diseases mentioned below.

Where subsequently or above the term “use” is mentioned (as verb or noun) (relating to the use of a compound of the formula (I) or a pharmaceutically acceptable salt thereof), this includes any one or more of the following embodiments of the invention, respectively: the use in the treatment of a protein kinase dependent disease, the use for the manufacture of pharmaceutical compositions for use in the treatment of a protein kinase dependent disease, methods of use of one or more compounds of the formula (I) in the treatment of a protein kinase dependent disease, the use of pharmaceutical preparations comprising one or more compounds of the formula (I) for the treatment of a protein kinase dependent disease, and one or more compounds of the formula (I) for use in the treatment of a protein kinase dependent disease, as appropriate and expedient and if not stated otherwise. In particular, diseases to be treated and are thus preferred for “use” of a compound of formula (I) are selected from protein kinase dependent (“dependent” meaning also “supported”, not only “solely dependent”) diseases mentioned herein, especially proliferative diseases mentioned herein, more especially any one or more of these or other diseases that depend on one or more of PKB, ALK, S6K1 and RET or any combinations of two or more of these, or a mutant of any one or more of these, and a compound of the formula (I) can therefore be used in the treatment of a kinase dependent disease, especially a disease depending on one or more of the kinases mentioned above and below, where (especially in the case of aberrantly highly-expressed, constitutively activated and/or mutated kinases) said kinase-dependent disease is dependent on the activity of one or more of the said kinases or the pathways they are involved.

The compounds of formula (I) have valuable pharmacological properties and are useful in the treatment of protein kinase dependent diseases, e.g., as drugs to treat proliferative diseases.

### PREFERRED EMBODIMENTS OF THE INVENTION

With the groups of preferred compounds of formula (I) mentioned hereinafter, definitions of substituents from the general definitions mentioned hereinbefore may reasonably be used, e.g., to replace more general definitions with more specific definitions or especially with definitions characterized as being preferred. The invention relates especially to a compound of the formula (I)

wherein
each of x and y is, independently of the other, 0 or 1;
R₁ is an organic moiety that can be bound to nitrogen;
X is C═O (especially preferred) or C═S with the proviso that then the dashed line bonding X to N is absent, so that X is bound to the adjacent N via a single bond and with the proviso that then y is 1 and R is hydrogen or an organic moiety that can be bound to nitrogen; or
X is (CR₇), wherein R₇ is hydrogen or an organic or inorganic moiety with the proviso that then the dashed line bonding X to N is a bond, so that X is bound to the adjacent N via a double bond, and with the proviso that then y is zero or y is 1 and then —R is →O; and
each of R₂, R₃, R₄; R₅ and R₆, independently of the others, is an organic moiety or hydrogen or an inorganic moiety, with the proviso that R₃ cannot be unsubstituted phenyl unless R₁ is phenyl substituted with a heterocyclic ring;
   or a pharmaceutically acceptable salt thereof.

More preferred is a compound of the formula (I),

wherein
each of x and y is, independently of the other, 0 or 1;
R₁ is substituted or unsubstituted aryl or heteroaryl, especially phenyl, which is substituted with up to 4 substituents, preferably up to 3 substituents, where the substituents are the same or different and are independently selected from halo, e.g., F or Cl; C₁-C₇lower alkyl, which may be unsubstituted or substituted with halo, especially methyl, ethyl, propyl or trifluoromethyl; cyano; cyano-lower alkyl, e.g., cyanomethyl, cyanoethyl, or cyanopropyl; lower alkoxy; amino; amino-lower alkyl; amino-lower alkoxy; amino-lower alkyl sulfanyl or thiol-lower alkyl, wherein the amino group can be mono- or di-substituted, e.g., —(C₁-C₇)ₘNR₈R₉ or —O—(C₁-C₇)ₘNR₈R₉,
   wherein
m is 0 or 1;
R₈ and R₉ can be the same or different and are independently H; lower alkyl, e.g., methyl, ethyl or propyl; lower cycloalkyl, e.g., cyclopropyl, or
R₈ and R₉, together with the N atom, form a 3- to 8-membered heterocyclic ring containing 1-4 nitrogen, oxygen or sulfur atoms, e.g., azetidinyl, pyrrolidinyl, piperidino, morpholinyl, imidazolinyl, imidazolinyl-ethyl, piperazinyl or lower alkyl-piperazinyl; amino-carbonyl-lower alkyl, e.g., R₈R₉—N—C(O)—CH₂—, wherein R₈ and R₉ are as defined above; heterocyclyl; heterocyclyl-lower alkyl; heterocyclyl-lower alkoxy or heterocyclyl-lower alkanesulfanyl, wherein the heterocyclyl is a 3- to 8-membered heterocyclic ring containing 1-4 nitrogen, oxygen or sulfur atoms, e.g., imidazolyl, Imidazolinyl, pyrrolidinyl, morpholinyl, azetidinyl, pyridyl, piperidino, piperidyl, piperazinyl or lower alkyl-piperazinyl; substituted or unsubstituted amide; amide-lower alkyl, e.g., —CH₂—CH(NH₂)—C(O)—NH₂), wherein alkyl may be linear, or cyclic, e.g., cyclopropylene; and the alkyl in any of the substituents above may optionally be substituted with —NR₈R₉, wherein R₈ and R₉ are as defined above;
X is C═O or C═S, with the proviso that then the dashed line bonding X to N is absent, so that X is bound to the adjacent N via a single bond and with the proviso that then y is 1 and R is hydrogen or an organic moiety that can be bound to nitrogen, or
X is (CR₇), wherein R₇ is hydrogen or an organic moiety, such as C₁-C₇ lower alkyl; amino or amino lower alkyl, wherein the alkyl may be unsubsbtuted or substituted with halo, e.g., methyl, ethyl, propyl, trifluoromethyl; lower alkoxy, e.g., methoxy; or cycloalkyl, e.g., cyclopropyl, with the proviso that then the dashed line bonding X to N is a bond, so that X is bound to the adjacent N via a double bond, and with the proviso that then y is zero, or y is 1 and then —R is →O;
R₂ is hydrogen;
R₃ is halo, e.g., Cl or F; unsubstituted or substituted C₅-C₁₄heterocyclyl, e.g., thienyl, benzo[1,3]dioxolo, indolyl, benzofuranyl or pyridiyl; unsubstituted or substituted C₅-C₁₄-aryl, e.g., phenyl or phenyl substituted with up to 4 substituents, preferably up to 3 substituents, which are the same or different and are selected from halo, e.g., Cl or F; hydroxy; C₁-C₄lower alkoxy, e.g., methoxy; lower alkyl, e.g., methyl; or —(C₁-C₄)ₘNR₈R₉,
   wherein
m is 0 or 1; and
R₈ and R₉, are as defined above, e.g., piperazinyl, methylpiperazinyl, morpholinyl or pyrrolidinyl; a heteroaryl being unsubstituted or substituted by one or more, especially 1-3 substituents independently selected from the group consisting of the substituents defined above under “substituted”, especially being pyridyl (or an N-oxide of pyridyl) which is unsubstituted or substituted by one to two radicals selected from the group consisting of lower alkyl, e.g., methyl; lower alkoxy, e.g., methoxy; halo, e.g., fluoro; or —NR₈R₉,
   wherein
      R₈ and R₉ can be the same or different and are independently H; lower alkyl, e.g., methyl, ethyl or propyl; lower cycloalkyl, e.g., cyclopropyl, or
      R₈ and R₉ can, with the N atom, form a 3- to 8-membered heterocyclic ring containing 1-4 nitrogen, oxygen or sulfur atoms, e.g., azetidinyl, pyrrolidinyl, piperidino, morpholinyl, imidazolinyl, piperazinyl or lower alkyl-piperazinyl;
R₄ is hydrogen or halo, e.g., fluoro or chloro;
R₅ is hydrogen; and
R₆ is hydrogen, amino, amino-lower alkyl or alkylamido, e.g., methylamido —NHC(O)CH₃, with the proviso that R₃ cannot be unsubstituted phenyl unless R₁ is phenyl substituted with an heterocyclic ring;
   or a pharmaceutically acceptable salt thereof, as such or especially for use in the diagnostic or therapeutic treatment of a warm-blooded animal, especially a human.

Especially preferred is a compound of the formula (I),

wherein
each of x and y is, independently of the other, 0 or 1;
R₁ is substituted or unsubstituted aryl or heteroaryl, especially phenyl, which is substituted with up to 4 substituents, preferably up to 3 substituents, where the substituents are the same or different and are independently selected from halo, e.g., F or Cl; C₁-C₇lower alkyl which may be unsubstituted or substituted with halo, especially methyl, ethyl, propyl or trifluoromethyl; cyano; cyano-lower alkyl, e.g., cyanomethyl, cyanoethyl or cyanopropyl; amino; amino-lower alkyl, wherein the amino group can be mono- or di-substituted, e.g., —(C₁-C₇)ₘNR₈R₉ or —O—(C₁-C₇)ₘNR₈R₉,
   wherein
      m is 0 or 1;
      R₈ and R₉ can be the same or different and are independently H; lower alkyl, e.g., methyl, ethyl or propyl; lower cycloalkyl, e.g., cyclopropyl, or
      R₈ and R₉, together with the N atom, form a 3- to 8-membered heterocyclic ring containing 1-4 nitrogen, oxygen or sulfur atoms, e.g., imidazolinyl, imidazolinyl-ethyl, piperazinyl or lower alkyl-piperazinyl; amino-carbonyl-lower alkyl, e.g., R₈R₉—N—C(O)—CH₂—, wherein R₈ and R₉ are as defined above; heterocyclyl; heterocyclyl-lower alkyl; heterocyclyl-lower alkoxy or heterocyclyl-lower alkanesulfanyl, wherein the heterocyclyl is a 3- to 8-membered heterocyclic ring containing 1-4 nitrogen, oxygen or sulfur atoms, e.g., imidazolinyl, imidazolinyl, imidazolinyl-ethyl, piperazinyl or lower alkyl-piperazinyl; substituted or unsubstituted amide; amide-lower alkyl, e.g., —CH₂—CH(NH₂)—C(O)—NH₂), wherein alkyl may be linear or cyclic, e.g., cyclopropylene; and the alkyl in any of the substituents above may optionally be substituted with —NR₈R₉, wherein R₈ and R₉ are as defined above;
X is (CR₇), wherein R₇ is hydrogen; lower alkyl, e.g., methyl or ethyl; amino alkyl or amino, with the proviso that then the dashed line bonding X to N is a bond, so that X is bound to the adjacent N via a double bond, and with the proviso that then y is zero, or y is 1 and then —R is →O;
R₂ is hydrogen;
R₃ is halo, e.g., Cl or F; unsubstituted or substituted C₅-C₁₄heterocyclyl, e.g., thienyl, benzo[1,3]dioxolo, indolyl, benzofuranyl or pyridiyl; unsubstituted or substituted C₅-C₁₄aryl, e.g., phenyl or phenyl substituted with up to 4 substituents, preferably up to 3 substituents, which are the same or different and are selected from halo, e.g., Cl or F; hydroxy; C₁-C₄lower alkoxy, e.g., methoxy; lower alkyl, e.g., methyl; or —(C₁-C₄)ₘNR₈R₉,
wherein
   m is or 1;
   R₈ and R₉ are as defined above, e.g., piperazinyl, methylpiperazinyl, morpholinyl or pyrrolidinyl;
R₄ is hydrogen or halo, e.g., fluoro or chloro;
R₅ is hydrogen; and
R₆ is hydrogen, with the proviso that R₃ cannot be unsubstituted phenyl unless R₁ is phenyl substituted with an heterocyclic ring;
   or a pharmaceutically acceptable salt thereof, as such or especially for use in the diagnostic or therapeutic treatment of a warm-blooded animal, especially a human.

Also preferred are pharmaceutical preparations comprising an imidazoquinoline compound of the formula (I), or a pharmaceutically acceptable salt thereof, especially for the treatment of a protein kinase dependent disease; a process for the manufacture of the novel imidazoquinoline compounds of the formula (I), or a pharmaceutically acceptable salt thereof and novel starting materials and Intermediates for their manufacture. Especially preferred is the use of a compound of formula (I), or a pharmaceutically acceptable salt thereof, in the manufacture of a pharmaceutical preparation for the treatment of a protein kinase dependent disease.

Also preferred is a compound of the formula (I), or a pharmaceutically acceptable salt thereof, as shown above for use in the treatment of a protein kinase dependent disease, especially one depending on PKB, ALK, S6K1 or RET and (especially aberrantly highly expressed or activated) PKB, ALK, S6K1 or RET-dependent disease or disease dependent on the activation of the PKB, ALK, S6K1 or RET pathways or disease dependent on any two or more of the kinases just mentioned.

Especially preferred is a compound of the formula (I), or a pharmaceutically acceptable salt thereof,

wherein
X is C═O; and
the other moieties are as defined under formula (I),
   for use in the diagnostic or therapeutic treatment of a warm-blooded animal, especially a human.

In an alternative embodiment, the present invention is directed to a method of treating a protein kinase dependent disease, especially one depending on PKB, ALK, S6K1 or RET and (especially aberrantly highly expressed or activated) PKB, ALK, S6K1 or RET-dependent disease or disease dependent on the activation of the PKB, ALK, S6K1 or RET pathways or disease comprising administering a compound according to formula (I),

wherein
each of x and y is, independently of the other, 0 or 1;
R₁ is an organic moiety that can be bound to nitrogen;
X is C═O (especially preferred) or C═S with the proviso that then the dashed line bonding X to N is absent, so that X is bound to the adjacent N via a single bond and with the proviso that then y is 1 and R is hydrogen or an organic moiety that can be bound to nitrogen; or
X is (CR₇), wherein R₇ is hydrogen or an organic or inorganic moiety with the proviso that then the dashed line bonding X to N is a bond, so that X is bound to the adjacent N via a double bond, and with the proviso that then y is zero or y is 1 and then —R is →O; and
each of R₂, R₃, R₄; R₅ and R₆, independently of the others, is an organic moiety or hydrogen or an Inorganic moiety;
   or a pharmaceutically acceptable salt thereof.

Very preferred is a method of treating a protein kinase dependent disease comprising administering a compound of formula (I), where the disease to be treated is a proliferative disease, preferably a benign or especially malignant tumor, more preferably carcinoma of the brain, kidney, liver, adrenal gland, bladder, breast, stomach (especially gastric tumors), ovaries, colon, rectum, prostate, pancreas, lung, vagina, thyroid, sarcoma, glioblastomas, multiple myeloma or gastrointestinal cancer, especially colon carcinoma or colorectal adenoma; or a tumor of the neck and head, an epidermal hyperproliferation, especially psoriasis, prostate hyperplasia, a neoplasia, especially of epithelial character, preferably mammary carcinoma, or a leukemia. Also preferred is a method of treating metabolic diseases, e.g., type II diabetes, obesity, hyperlipidemia and atherosclerosis.

Most preferred is the use in accordance with the present invention of a compound of the formula (I), or a pharmaceutically acceptable salt thereof, as exemplified hereinbelow under “Examples”.

RET kinase inhibition is determined as follows: The baculovirus donor vector pFB-GSTX3 is used to generate a recombinant baculovirus that expresses the amino acid region 658-1072 (Swiss Prot No. Q9BTB0) of the intra-cytoplasmic kinase domain of human RET-Men2A which corresponds to the wild-type kinase domain of RET (wtRET) and RET-Men2B, which differs from the wtRET by the activating mutation in the activation loop M918T. The coding sequences for the cytoplasmic domain of wtRET and RET-Men2B are amplified by PCR from the plasmids pBABEpuro RET-Men2A and pBABEpuro RET-Men2B, respectively which are received from Dr. James Fagin, College of Medicine, University of Cincinnati. The amplified DNA fragments and the pFB-GSTX3 vector are made compatible for ligation by digestion with SaII and KpnI. Ligation of these DNA fragments results in the baculovirus donor plasmid pFB-GX3-RET-Men2A and pFB-GX3-RET-Men2B, respectively.

Production of virus: Transfer vectors containing the kinase domains are transfected into the DH10Bac cell line (GIBCO) and plated on selective agar plates. Colonies without insertion of the fusion sequence into the viral genome (carried by the bacteria) are blue. Single, white colonies are picked and viral DNA (bacmid) isolated from the bacteria by standard plasmid purification procedures. Sf9 cells or Sf21 (American Type Culture Collection) cells are then transfected in 25 cm² flasks with the viral DNA using Cellfectin reagent.

Determination of small scale protein expression in Sf9 cells: Virus-containing media is collected from the transfected cell culture and used for Infection to increase its titer. Virus-containing media obtained after two rounds of infection is used for large-scale protein expression. For large-scale protein expression 100 cm² round tissue culture plates are seeded with 5×10⁷ cells/plate and infected with 1 mL of virus-containing media (approximately 5 MOls). After 3 days, the cells are scraped off the plate and centrifuged at 500 rpm for 5 minutes. Cell pellets from 10-20, 100 cm² plates, are re-suspended in 50 mL of ice-cold lysis buffer (25 mM tris-HCl, pH 7.5, 2 mM EDTA, 1% NP-40, 1 mM DTT, 1 mM P MSF). The cells are stirred on ice for 15 minutes and then centrifuged at 5,000 rpms for 20 minutes.

Purffication of GST-tagged proteins: The centrifuged cell lysate is loaded onto a 2 mL glutathione-sepharose column (Pharmacia) and washed 3× with 10 mL of 25 mM tris-HCl, pH 7.5, 2 mM EDTA, 1 mM DTT, 200 mM NaCl. The GST-tagged proteins are then eluted by 10 applications (1 mL each) of 25 mM tris-HCl, pH 7.5, 10 mM reduced-glutathione, 100 mM NaCl, 1 mM DTT, 10% glycerol and stored at −70° C.

Measure of enzyme activity: Tyrosine protein kinase assays with either purified GST-wtRET or GST-RET-Men2B protein are carried out in a final volume of 30 μL containing 15 ng of either GST-wtRET or GST-RET-Men2B protein, 20 mM tris-HCl, pH 7.5, 1 mM MnCl₂, 10 mM MgCl₂, 1 mM DTT, 3 μg/mL poly(Glu, Tyr) 4:1, 1% dimethyl sulfoxide (DMSO), 2.0 μM ATP (γ-[³³P]-ATP 0.1 μCi). The activity is assayed in the presence or absence of inhibitors, by measuring the Incorporation of ³³P from [γ³³P] ATP into poly(Glu, Tyr) 4:1. The assay is carried out in 96-well plates at ambient temperature for 15 minutes under conditions described below and terminated by the addition of 20 μL of 125 mM EDTA. Subsequently, 40 μL of the reaction mixture are transferred onto Immobilon-PVDF membrane (Millipore) previously soaked for 5 minutes with methanol, rinsed with water, then soaked for 5 minutes with 0.5% H₃PO₄ and mounted on vacuum manifold with disconnected vacuum source. After spotting all samples, vacuum is connected and each well-rinsed with 200 μL 0.5% H₃PO₄. Membranes were removed and washed 4× on a shaker with 1.0% H₃PO₄, once with ethanol. Membranes are counted after drying at ambient temperature, mounting in Packard TopCount 96-well frame, and addition of 10 μL/well of Microscint™ (Packard). IC₅₀ values are calculated by linear regression analysis of the percentage inhibition of each compound in duplicate, at 4 concentrations (usually 0.01, 0.1, 1 and 10 μM). One unit of protein kinase activity is defined as 1 nmole of ³³P ATP transferred from [γ³³P] ATP to the substrate protein/minute/mg of protein at 37° C.

IC₅₀ calculations

| | |
|---|---|
| input | 3 × 4 μL stopped assay on Immobilon |
| | membrane, not washed |
| background (3 wells) | assay with H₂O instead of enzyme |
| positive control (4 wells) | 3% DMSO instead of compound |
| bath control (1 well) | no reaction mix |

IC₅₀ values are calculated by logarithmic regression analysis of the percentage inhibition of each compound at 4 concentrations (usually 3- or 10-fold dilution series starting at 10 μM). In each experiment, the actual inhibition by reference compound is used for normalization of IC₅₀ values to the basis of an average value of the reference inhibitor

Normalized IC₅₀=measured IC₅₀ average ref. IC₅₀/measured ref. IC₅₀

### Example: Reference Inhibitor in Experiment 0.4 μM, Average 0.3 μM

Test Compound in Experiment 1.0 μM, Normalization: 0.3/0.4=0.75 μM

For example, staurosporine or a synthetic staurosporine derivative are used as reference compounds.

Using this protocol, the compounds of the formula (I) are found to show IC₅₀ values for RET inhibition in the range from 0.001-20 μM, preferably in the range from 0.01-2 μM.

The efficacy of the compounds of formula (I) as inhibitors of PKB kinase activity can be demonstrated as follows: To fully activate PKB, the enzyme is exposed to catalytic amounts of PDK1. GST-PKB [100 ng, specific activity (SA): 0.2 nmole/mg/min.] is incubated for 30 minutes at room temperature (RT) with purified recombinant GST-PDK1 (1 ng, SA: 2 nmole/min./mg). The activation is performed as follows: 0.1 μg of GST-PDK1 (0.05 μL) and 10 μg of GST-PKB (0.45 μL) are mixed in a total volume of 0.75 μL containing 15 μM ATP, 3 mM MgCl₂, 20 mM hepes (pH 7.6) for 30 minutes at RT. The reaction is subsequently stopped by adding 0.25 μL containing 30% gycerol (w/w) and 0.06 μL of 500 mM EDTA. 100-500 ng (0.01-0.05 μL) activated GST-PKB is incubated in a final volume of 30 μL with 10 μM of the RRPRTRSFS peptide, 10 mM Mg-acetate, 50 mM MOPS (PH 7.5), 1 mM DTT and 300 μg/mL BSA, 20 μM ATP (0.1 μCi γ-³³P-ATP). The reaction is carried out for 30 minutes at RT in the presence of either 1% DMSO or the text compound of the formula (I) at the required concentration in 1% DMSO. The reaction is terminated by the addition of 20 μL 125 mM EDTA. Thirty (30) μL of each sample is spotted onto P81 Whatman and the paper squares processed. See Ferrari and Thomas, Methods Enzymol, Vol. 200, pp. 159-169 (1991). IC₅₀ values are calculated by linear regression analysis of the percentage inhibition of each compound in duplicate. IC₅₀ values for compounds of the formula (I) are in the range from 0.005-100 μM, for preferred compounds between 0.01 μM and 2 μM.

The efficacy of the compounds of formula (f) as inhibitors of S6K1 activity can be demonstrated as follows: The DNA fragment encoding ORF of human S6K1 (+28-1605, Genbank NM_{—}003161) is amplified from human muscle cDNA library and modified by PCR to generate a C-terminal truncated form (amino acid region 1-421) with an activating mutation of T412E, hereinafter S6K1ΔCT/T412E. The DNA fragment encoding S6K1ΔCT/T412E is subcloned to the baculovirus donor vector pFastBacHT A to generate a recombinant baculovirus that expresses S6K1ΔCT/T412E. Transfer vectors for S6K1ΔCT/T412E are transfected into the DH10Bac cell line (GIBCO). After selection of white colonies, they are used for viral DNA (bacmid) preparation. Sf9 cells (American Type Culture Collection) cells are then transfected with the viral DNA using Cellfectin reagent. Virus-containing media is collected from the transfected cell culture and is used for infection to increase its titer. For large-scale protein expression, Sf9 cells are co-infected with S6K1ΔCT/T412E and GST-PDK1 virus-containing media with MOI of 2.25 and 1, respectively. After 3 days, the cells expressing the recombinant enzyme are resuspended in the culture medium with a sterilized cell scraper and harvested by centrifugation. The cells are resuspended in 5 volumes of cell pellet of Extraction Buffer (50 mM MOPS pH 7.0, containing 300 mM NaCl, 0.05% Tween-20, 10 mM sodium pyrophosphate, 50 mM β-glycerophosphate, 0.2 mM cantharidic acid and 1× complete-EDTA protease inhibitor cocktail) and then disrupted with 15 strokes of a Dounce homogenizer followed by sonication on ice. The homogenate is centrifuged at 4° C. for 60 minutes at 48,000×g. The supernatant is clarified with a 5 μm filter and subjected to purification with TALON Superflow Resin (Clontech). First, 1 mL of TALON resin per 5 mL of the supernatant was equilibrated with the Extraction Buffer and resuspended in the supernatant followed by gentle agitation for 20 minutes at RT. After centrifugation at 700×g for 5 minutes, supernatant is removed. The resin is washed twice by adding 10 bed volumes of the Extraction Buffer, agitating for 10 minutes at RT, centrifugating at 700×g for 5 minutes, and discarding the supernatant. The resin is then resuspended with the same volume of the Extraction Buffer and transferred to an disposable chromatography column. The packed column is washed once with 10 bed volumes of the Extraction Buffer and additionally with the same volume of Wash Buffer (5 mM imidazole in the Extraction Buffer). Then, the His-tagged S6K1ΔCT/T412E is eluted with four bed volumes of Elution Buffer (150 mM imidazole in the Extraction Buffer).

Measure of enzyme activity: The kinase assay with purified S6K1ΔCT/T412E is performed with TR-FRET (Time-Resolved Fluorescene Resonance Energy Transfer) technology. First, ATP-dependent phosphorylation of the biotinylated substrate peptide corresponding to residues 229-242 of S6 ribosomal protein (Biot-AKRRRLSSLRASTS) is carried out for 1 hour at 37° C. in 382-well plates under humidified air. The kinase reaction mixture of the final volume of 9 μL contains 2.57 ng of purified S6K1ΔCT/T412E protein, 50 mM MOPS (pH 7.0), 0.1 mM EGTA, 0.01 mM Na₃VO₄, 0.05% Tween-20, 0.01% BSA, 1 mM DTT, 10 mM MgCl₂, 3 μM ATP, 0.5 μM biotinylated substrate peptide, 1% of DMSO or the text compounds of the formula (I) at the required concentration in 1% DMSO. For measurement of background, 20 mM of EDTA is added to the reaction mixture. The kinase reaction is terminated by addition of 6 μL of Detection Mixture [50 mM MOPS (pH 7.0), 25 mM EGTA, 200 mM NaCl, 0.05% Tween-20, 0.01% BSA, 5 μg/mL SureLight Allophycocyanin-streptavidin (Perkin Elmer), 2 nM LANCE Eu-W1024 anti-rabbit IgG (Perkin Elmer), 0.2 μg/mL Phospho-S6 Ribosomal Protein (Ser235/236) Antibody (Cell Signaling Technology)] and the plates are incubated for 2 hours. Phosphorylated peptide indicative of kinase activity is quantitated by exciting the sample at 330 nm and comparing donor emission at 615 nm and acceptor emission at 665 nm by 2101 EnVision HTS multilabel plate reader (Perkin Elmer). The ratio of acceptor counts divided by donor counts is used as a normalized parameter for both signal and background. IC₅₀ values are calculated by linear regression analysis of the percentage inhibition of each compound in triplicate, at 7 concentrations (usually 0.01, 0.001, 0.03, 0.1, 0.3, 1 and 10 μM).

The efficacy of the compounds of formula (I) as inhibitors of ALK tyrosine kinase activity can be demonstrated as follows: The inhibition of ALK tyrosine kinase activity can be demonstrated using known methods, e.g., using the recombinant kinase domain of the ALK in analogy to the VEGF-R kinase assay. See Wood et al., Cancer Res, Vol. 60, No. 8, pp. 2178-2189 (2000). In vitro enzyme assays using GST-ALK protein tyrosine kinase are performed in 96-well plates as a filter binding assay in 20 mM tris-HCl, pH=7.5, 3 mM MgCl₂, 10 mM MnCl₂, 1 mM DTT, 0.1 μCi/assay (=30 μL) [γ-³³P]-ATP, 2 μM ATP, 3 μg/mL poly (Glu, Tyr 4:1) Poly-EY (Sigma P-0275), 1% DMSO, 25 ng ALK enzyme. Assays are incubated for 10 minutes at ambient temperature. Reactions are terminated by adding 50 μL of 125 mM EDTA, and the reaction mixture is transferred onto a MAIP Multiscreen plate (Millipore, Bedford, Mass., USA), previously wet with methanol, and re-hydrated for 5 minutes with H₂O. Following washing (0.5% H₃PO₄), plates are counted in a liquid scintillation counter. IC₅₀ values are calculated by linear regression analysis of the percentage inhibition. Compared with the control without inhibitor, the compounds of formula (I) inhibit the enzyme activity by 50% (IC₅₀), e.g., in a concentration of from 0.001-0.5 μM, especially from 0.1-0.8 μM.

The compounds of formula (I) that inhibit the protein kinase activities mentioned, especially tyrosine and/or the serine/threonine protein kinases mentioned above, can therefore be used in the treatment of protein kinase dependent diseases, especially diseases depending on PKB, ALK, S6K1 or RET and (especially aberrantly highly-expressed or activated) PKB, ALK, S6K1 or RET-dependent disease, or disease dependent on the activation of the PKB, ALK, S6K1 or RET pathways or any combination of two or more of the mentioned kinases.

Protein kinase dependent diseases are especially proliferative diseases, preferably a benign or especially malignant tumor, more preferably carcinoma of the brain, kidney, liver, adrenal gland, bladder, breast, stomach (especially gastric tumors), ovaries, colon, rectum, prostate, pancreas, lung, vagina, thyroid, sarcoma, glioblastomas, multiple myeloma or gastrointestinal cancer, especially colon carcinoma or colorectal adenoma, or a tumor of the neck and head, an epidermal hyperproliferation, especially psoriasis, prostate hyperplasia, a neoplasia, especially of epithelial character, preferably mammary carcinoma, or a leukemia, especially as far as c-Met is involved. They are able to bring about the regression of tumors and to prevent the formation of tumor metastases and the growth of (also micro)metastases. In addition, they can be used in epidermal hyperproliferaflon, e.g., psoriasis; in prostate hyperplasia; in the treatment of neoplasias, especially of epithelial character, e.g., mammary carcinoma; and in leukemias. It is also possible to use the compounds of formula (I) in the treatment of diseases of the immune system insofar as several or, especially, Individual tyrosine protein kinases and/or (further) serine/threonine protein kinases are involved; furthermore, the compounds of formula (I) can be used also in the treatment of diseases of the central or peripheral nervous system where signal transmission by at least one tyrosine protein kinase and/or (further) serine/threonine protein kinase is involved.

There are also experiments to demonstrate the antitumor activity of compounds of the formula (I) in vivo.

Female Balb/c hairless mice with subcutaneously (s.c.) transplanted human bladder tumors T24 can be used to determine the anti-tumor activity. On day 0, with the animals under peroral forene narcosis, approximately 25 mg of a solid tumor are placed under the skin on the animals' left flank and the small incised wound is closed by means of suture clips. On day 6 after the transplantation, the mice are divided at random into groups of 6 animals and treatment commences. The treatment is carried out for 15 days with peroral, intravenous or intraperitoneal administration once daily (or less frequently) of a compound of formula (I) in DMSO/Tween80/sodium chloride solution in the various doses. The tumors are measured twice a week with a slide gauge and the volume of the tumors is calculated.

As an alternative to cell line A-431, other cell lines may also be used in the same manner, e.g.,
The MDA-MB 468 breast adenocarcinoma cell line [ATCC No. HTB 132; see also In Vitr, Vol. 14, pp. 911-915 (1978)];
The MDA-MB 231 breast carcinoma cell line [ATCC No. HTB-26; see also In Vitro, Vol. 12, p. 331 (1976)];
The Colo 205 colon carcinoma cell line [ATCC No. CCL 222; see also Cancer Res, Vol. 38, pp. 1345-1355 (1978)];
The DU145 prostate carcinoma cell line DU 145 [ATCC No. HTB 81; see also Cancer Res, Vol. 37, pp. 4049-4058 (1978)];
The PC-3 prostate carcinoma cell line PC-3 [especially preferred; ATCC No. CRL 1435; see also Cancer Res, Vol. 40, pp. 524-534 (1980)].
The A549 human lung adenocarcinoma [ATCC No. CCL 185; see also Int J Cancer, Vol. 17, pp. 62-70 (1976)];
The NCI—H596 cell line [ATCC No. HTB 178; see also Science, Vol. 246, pp. 491-494 (1989)]; and
The pancreatic cancer cell line SUIT-2 [see Tomioka et al., Cancer Res, Vol. 61, pp. 7518-7524 (2001)].

The compounds of the formula (I) can be prepared according to the methods:

In one preferred embodiment, a compound of formula (I) is prepared by reacting a compound of the formula (II)

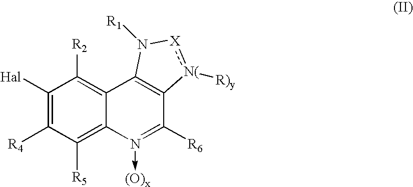

with a boronic acid,
wherein
Hal refers to halogen preferably bromine; and
x, y, X, R₁, R₂, R₄, R₅, R and R₆ are as defined above; and
   if desired, transforming an obtainable compound of formula (I) into a different compound of formula (I), transforming a salt of an obtainable compound of formula (I) into the free compound or a different salt, or an obtainable free compound of formula (I) into a salt; and/or separating an obtainable mixture of isomers of compounds of formula (I) into the individual isomers.

In the following, more detailed description of the preferred process conditions, x, y, R₁, R₂, R₃, R₄, R₅, R₆, R₇, X and R have the meanings given for compounds of the formula (I), if not indicated otherwise.

### Starting Materials

A compound of formula (II) of the first preferred embodiment is prepared by reacting a compound of formula (XVI)

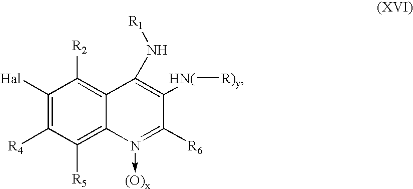

wherein
x, y, R₁, R₂, R₄, R₅ and R₆ are as mentioned for a compound of the formula (I); and
R are as defined below under a), b) or c), respectively,
   a) for the manufacture of a compound of the formula II wherein X is C═O and the dashed line in formula (I) bonding X to N is absent, y is 1 and R is hydrogen or an organic moiety that can be bound to nitrogen, with an active derivative of a compound of the formula (III)

A-X-A (III),
wherein
   X is C═O; and
   each A, independently of the other, is a carbonyl-activating group;
b) for the manufacture of a compound of the formula (II), wherein X is C═S and the dashed line in formula (I) bonding X to N is absent, y is 1 and R is hydrogen or an organic moiety that can be bound to nitrogen, with CS₂ or Cl—C(═S)—Cl; or
c) for the manufacture of a compound of the formula (II), wherein X is (CR₇), wherein R₇ is hydrogen or an organic or inorganic moiety with the proviso that then the dashed line bonding X to N is a bond, so that X is bound to the adjacent N via a double bond, with an activated derivative of a compound of formula (IVa), (IVb) or (IVc) or a derivative of one of these compounds:

R₇—COOH (IVa),

R₇—N (IVb), or

R₇—CHO (IVc),
wherein R₇ is hydrogen, an organic or inorganic moiety, e.g., C₁-C₇lower alkyl, amino or amino-lower alkyl;
   wherein functional groups which are present in the starting compounds in processes a) to c) and are not intended to take part in the reaction, are present in protected form if necessary, and protecting groups that are present are cleaved, wherein said starting compounds may also exist in the form of salts provided that a salt-forming group is present and a reaction in salt form is possible.

A compound of the formula (II),

wherein
R is hydrogen; and
y is 1 is preferably prepared by hydrogenation of a compound of the formula (V)

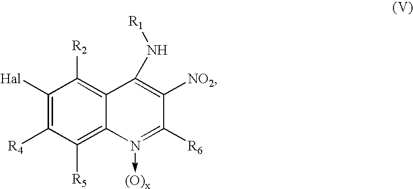

wherein the substituents and symbols are defined as for compounds of the formula (I) (x is preferably zero) in the presence of an appropriate catalyst, e.g., a skeleton based catalyst, such as Raney-Ni with hydrogen in an appropriate solvent, e.g., an alcohol, such as methanol; at preferred temperatures between 0° C. and 50° C., e.g., at RT.

The corresponding compounds of the formula (II), wherein R is an organic moiety that can be bound to nitrogen, especially a carbon-bound one, can be prepared by reaction of a compound of formula (II),

wherein
R is hydrogen; and
y is 1 (see preceding paragraph), with a compound of the formula (VI)

R-L (VI),

wherein
R is an organic moiety bound to L via a carbon atom; and
L is a leaving group, especially halo, such as chloro, bromo or iodo; or arylsulfonyl, e.g., toluenesulfonyl in an appropriate solvent; preferably in the presence of a tertiary nitrogen base, such as pyridine or triethylamine.

Alternatively, a compound of the formula (II),

wherein
R is hydrogen; and
y is 1 can be reacted with an aldehyde of the formula (VI*) or (VI**)

R*—CHO (VI*) or

R*—CO—R** (VI**),
wherein R* and R** are the same or different and each is as an organic moiety bound to the moiety —CHO via a carbon atom; followed by reduction of the resulting enamine with an appropriate reductant, e.g., a complex hydride, such as an alkalimetal cyanoborohydride, e.g., sodium-cyanoborohydride, e.g., in the same solvent and at temperatures between −10° C. and 40° C., e.g., at 10° C., the total reaction summing up to reductive amination.

A compound of formula (V) is preferably prepared by reacting a compound of the formula (VII)

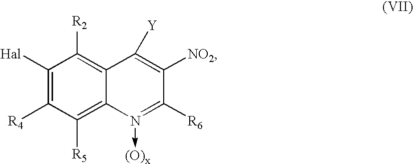

wherein
Y is halo, especially chloro; and
the other moieties and symbols have the meanings indicated for compounds of the formula (I) (x is preferably zero), with a compound of the formula (VIII)

R₁—NH₂ (VIII),
wherein R₁ is as defined for a compound of the formula (I) in an appropriate solvent; preferably a lower alkylcarboxylic acid, such as acetic acid; at preferred temperatures between 10° C. and reflux temperature of the reaction mixture, e.g., between 20° C. and 140° C.

A compound of the formula (VII) can be prepared by reacting a compound of the formula (IX)

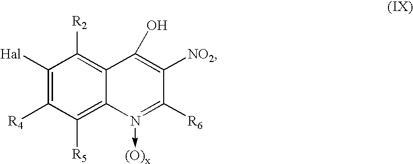

wherein the moieties and symbols have the meanings indicated for a compound of the formula (I) (x is preferably zero), with an inorganic acid halogenide, especially POCl₃ (preferably without solvent) at elevated temperatures, e.g., between 100° C. and 150° C. or under reflux.

A compound of the formula (IX) is known in the art, can be synthesized according to methods known in the art and/or is commercially-available. For example, it can be synthesized by reacting a compound of the formula (X)

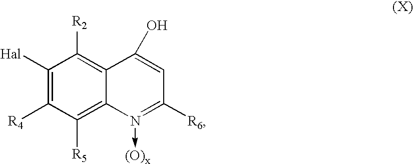

wherein the moieties and symbols have the meanings indicated for a compound of the formula (I) (x is preferably zero), with nitric acid (aqueous) at a preferred temperature between 50° C. and 100° C., e.g., at 85° C.

A compound of the formula (IX), can alternatively be synthesized by reacting a compound of the formula (XI)

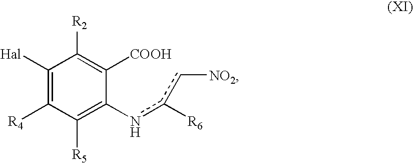

wherein the moieties and symbols have the meanings indicated for a compound of the formula (I), with an anhydride of a carbonic acid, especially acetic anhydride, preferably in the presence of an alkali metal salt of a carboxylic acid, e.g., potassium acetate, at a preferred temperature between 50-C and 150° C., e.g., at ca. 100-140° C.

A compound of the formula (XI) can be obtained, e.g., by converting a compound of the formula (XII)

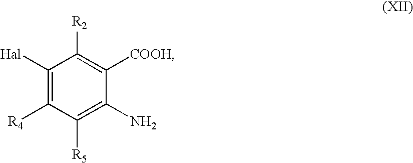

to the corresponding compound of the formula (XI) by reacting nitromethane in the presence of an alkali metal hydroxide, especially sodium hydroxide, at preferred temperatures between approximately 0° C. and 60° C., e.g., between 0° C. and RT; then pouring the product under cooling to approximately 0° C. into concentrated HCl and adding the compound of the formula (XII) and further concentrated HCl, subsequently allowing for further reaction at preferred temperatures between 0° C. and RT to result in the corresponding compound of formula (XI).

Other starting materials are either known in the art, can be prepared according to methods that are known in the art, e.g., in analogy to the methods described hereinabove or in the examples, and/or are commercially-available.

The present invention relates also to novel starting materials and/or intermediates and to processes for their preparation. The starting materials used and the reaction conditions selected are preferably those that result in the compounds described as being preferred.

Other starting materials are either known in the art, can be prepared according to methods that are known in the art, e.g., in analogy to the methods described hereinabove or in the examples, and/or are commercially-available.

The present invention relates also to novel starting materials and/or intermediates and to processes for their preparation. The starting materials used and the reaction conditions selected are preferably those that result in the compounds described as being preferred.

### DETAILED DESCRIPTION OF PREFERRED REACTION CONDITIONS

The reaction described under (a) preferably takes place under conditions known in the art, especially in an appropriate solvent, such as a halo-lower alkane, e.g., dichloromethane; or a lower alkylnitrile, such as acetonitrile and under elevated temperatures, preferably in the range from 40° C. to the reflux temperature of the reaction mixture, especially under reflux. In the compound of the formula (III), each A is, independently of the other, preferably halo, trichloromethyl, succinimido or 1-imidazolo. For example, if the compound of the formula (III) is trichloromethyl chloroformate, the reaction preferably takes place under anhydrous conditions in an appropriate aprotic solvent, e.g., a halogenated hydrocarbon, such as dichloromethane, at preferred temperatures between 0° C. and 50° C., e.g., at RT.

The reaction described under (b) with CS₂ or Cl—C(═S)—Cl preferably takes place in the presence of a base, especially a tertiary amine, such as tri-lower alkylamine, preferably triethylamine or pyridine; an alkalimetal carbonate or -bicarbonate, e.g., sodium bicarbonate; or a metal hydroxide, especially an alkali metal hydroxide, such as sodium or potassium hydroxide, in a polar organic solvent, especially an alcohol, at temperatures between 10° C. and the reflux temperature, more preferably between 20° C. and 100° C.

The reaction described under (c) preferably takes place in the presence of an active derivative of a compound of the formulae (IVa), (IVb) and (IVc) as solvent or other appropriate solvents or solvent mixtures at preferred temperatures between 30° C. and the reflux temperature of the reaction mixture, more preferably under reflux. An activated derivative of a compound of the formula (IVa) is especially a tri-lower alkyl orthoester of the carbonic acid of formula (IVa), especially a tri-ethyl derivative, such as triethylorthoformate or a tetramethyl derivative, such as tetramethyl orthocarbonate. Alternatively, the respective reactive derivative of an acid of the formula (IVa) is formed in situ, e.g., in the presence of polyphosphoric acid (also as solvent) at elevated temperatures, e.g., between 100° C. and 140° C. An activated derivative of a compound of formula (IVb) is especially a halo derivative, such as cyanogen bromide.

Compounds of formula (I) can be transformed into different compounds of formula (I).

Especially, the following transformations are of interest:

In compounds of the formula (I), wherein R₁ carries a cyano or cyano-lower alkyl substituent, this substituent can be converted into an aminomethyl or aminomethyl-lower alkyl group, respectively, by hydrogenation, e.g., with hydrogen in the presence of an appropriate catalyst, such as a Raney catalyst, especially Raney-Ni; In an appropriate solvent, such as an alcohol, especially methanol or ethanol; or a cyclic ether, such as tetrahydrofuran, or a mixture thereof, In the presence of ammonia, preferably at temperatures between 0° C. and 50° C., e.g., at RT.

In compounds of the formula (I), wherein R₁ carries a cyano or cyano-lower alkyl substituent or R₇ is any one of these substituents, this substituent can be converted into a N-hydroxyamidino or N-hydroxyamidino-lower alkyl group, respectively, by reaction with a hydroxylamine salt of an organic or inorganic acid, e.g., a hydroxylamine halogenide; in a polar solvent, e.g., a di-lower alkyl lower alkanolamide, especially dimethyl formamide; in the presence of water at preferred temperatures between 10° C. and 100° C., e.g., at 20-75° C.; in the presence of a base, especially an alkali metal carbonate, such as sodium carbonate.

In compounds of the formula (I), wherein R₁ is 2-haloaryl, e.g., 2-chlorophenyl; the halogen can be removed by hydrogenation with hydrogen in an appropriate solvent, e.g., in an alcohol, such as methanol; or a N,N-di-lower alkyl-loweralkanoylamide, such as dimethylformamide or a mixture thereof; and a catalyst, such as a noble metal on a carrier material, e.g., palladium on charcoal (Pd—C), at preferred temperatures between 0° C. and 50° C., e.g., at RT, to the corresponding compound wherein R₁ is aryl, e.g., phenyl.

In a compound of the formula (I), wherein a hydroxyamidino substituent is present, e.g., as mentioned in the last paragraph; this substituent can be converted into the corresponding amidino substituent by hydrogenation in the presence of an acid, such as HCl; and a catalyst, preferably a Raney metal catalyst, such as Raney-Ni; preferably at elevated temperatures, e.g., between 30° C. and 70° C., e.g., at 50° C.

Compounds of the formula (I), wherein x and y or one of them are zero can be converted into the corresponding N-oxide compounds (x, y or both=1, R=→O) by oxidation in the presence of a peroxide, especially a peroxybenzoic acid derivative, such as 3-chloroperoxybenzoic acid; in the presence of a base, e.g., an alkali metal carbonate, such as sodium carbonate; and in an appropriate solvent, e.g., a halogenated hydrocarbon, such as chloroform or methylene chloride.

A compound of the formula (I),

wherein
x is 1; and
R₆ is hydrogen,
   can be transformed into the corresponding compound,
   wherein
x is zero; and
R₆ is arylcarbonylamino,
   by reaction with the corresponding aryl isocyanate, especially benzoyl isocyanate; in an appropriate solvent, e.g., a halogenated hydrocarbon, such as methylene chloride or chloroform; preferably at elevated temperatures, e.g., under reflux.

A compound of the formula (I), wherein R₆ is arylcarbonylamino can be converted into the corresponding compound of the formula (I), wherein R₆ is amino by reaction with an alkali metal alcoholate in the corresponding alcohol, e.g., sodium methanolate in methanol, at elevated temperatures, e.g., under reflux.

A compound of the formula (I),

wherein
x is 1; and
R₆ is hydrogen,
   can be transformed into the corresponding compound,
   wherein
x is zero; and
R₆ is cyano,
   by reaction with an metal cyanide, e.g., an alkali metal cyanide, especially potassium cyanide; in the presence of a base, e.g., a tertiary nitrogen base, such as a tri-lower alkylamine, e.g., triethylamine; in a polar solvent, e.g., a di-lower alkyl alkanoylamide, such as dimethylformamide; at elevated temperatures, e.g., between 80° C. and 120° C., e.g., between 100° C. and 110° C.

Compound of formula (I),

wherein
X is CR₇; and
R₇ is NH₂,
   is prepared from the corresponding di-amino compound and cyanogen bromide in an appropriate solvent, e.g. ethanol; at temperatures between 0° C. and 50° C., e.g., RT.

A compound of formula (I),

wherein
X is CR₇; and
R₇ is OCH₃,
   is prepared from the corresponding di-amino compound and tetramethyl orthocarbonate in the presence of an appropriate solvent, e.g., acetic acid; at elevated temperatures, e.g., 75° C.

A compound of formula (I),

wherein
X is CR₇; and
R₇ is CF₃,
   is prepared from the di-amino compound and trifluoroacetic acid in the presence of an appropriate solvent, e.g., 4 N HCl; at elevated temperatures, e.g., 100° C.

A compound of formula (I),

wherein
X is CR₇; and
R₇ is CH₃,
   is prepared from the corresponding di-amino compound and triethylorthoacetate at elevated temperatures, e.g., 130° C.

A compound of formula (I),

wherein
X is CR₇; and
R₇ is lower alkyl,
   is prepared from the corresponding di-amino compound and the corresponding aldehyde using catalytic amounts of acetic acid in an appropriate solvent, e.g., DCM; at temperatures between 0° C. and 50° C., e.g., RT.

A compound of formula (I), wherein R₃ is unsubstituted or substituted aryl or heterocyclyl is prepared by reacting the Br-derivative and the corresponding boronic acid in the presence of bis(triphenylphosphine)palladium (II) dichloride, 1 M solution of sodium carbonate in an appropriate solvent, e.g., DMF at elevated temperatures, e.g., 100° C. This is a Pd catalyzed cross-coupling reaction of aryl, alkynyl or vinyl halides with aryl or vinyl boronic acids. See Suzuki, Tetrahedron Lett, Vol. 20, p. 3437 (1979); or J Am Chem Soc, Vol. 107 p. 972 (1985).

A compound of the formula (I),

wherein
x is 1; and
R₆ is hydrogen,
   can be transformed into the corresponding compound,
   wherein
x is zero; and
R₆ is halo,
   by reaction with an inorganic halogenide, e.g., POCl₃; in an appropriate solvent, e.g., a mixture of a di-lower alkyl alkanoylamide, such as dimethylformamide; and an aromatic hydrocarbon, e.g., toluene; at elevated temperatures, e.g., between 50° C. and 90° C.

A compound of the formula (I), wherein R₆ is halo can be converted into a compound of the formula (I), wherein R₆ is amino substituted by one or two moieties selected from the group consisting of lower alkyl, substituted lower alkyl moieties, aryl, cycloalkyl and mercapto-lower alkyl by reaction with the corresponding primary or secondary amine, respectively; in an appropriate solvent, e.g., an alcohol, especially methanol or 2-ethoxyethanol; at temperatures between 100° C. and 130° C. (if necessary in a sealed reaction vessel, e.g., a sealed tube).

A compound of the formula (I),

wherein
X is (CR₇); and
R₇ is halogen,
   can be obtained from the corresponding compound, wherein R₇ is hydrogen by reaction with the corresponding halogen succinimide, especially N-bromosuccinimide, in the presence of the corresponding iron(III)halogenide, especially FeBr₃; in the absence or presence of an appropriate solvent at elevated temperatures, preferably under reflux.

A compound of the formula (I),

swherein
X is (CR₇); and
R₇ is cyano,
   can be obtained from the corresponding compound, wherein R₇ is —CONH₂ by reaction with an inorganic acid halogenide, especially POCl₃; in an appropriate base, especially pyridine; preferably at elevated temperatures; more preferably between 25° C. and 80° C. Alternatively, the compound can be obtained from a compound of the formula (I), wherein R₇ is bromo (as obtainable in the last paragraph); by reaction in the presence of CuCN and a catalyst, especially tris(dibenzylideneacetone)dipalladium chloroform adduct and 1,1′-bis(diphenylphosphino)ferrocene and of tetraethylammonium cyanide; in an appropriate solvent, e.g., a cyclic ether, such as dioxane; at preferred temperatures (if necessary in a sealed tube) between 100° C. and 150° C., e.g., at 140° C.

A compound of the formula (I),

wherein
X is C═O;
y is 1; and
R is unsubstituted or substituted alkyl, especially lower alkyl,
   can be obtained by converting the corresponding compound of the formula (I), wherein R is H with a halogenide, especially iodide, such as lower alkyl iodide; in the presence of a strong base, especially an alkali metal hydride, e.g., sodium hydride; in an appropriate aprotic solvent, e.g., a N,N-di-lower alkyl-lower alkanoylamide; at preferred temperatures in the range from 0-50° C., e.g., at RT, into said compound.

A compound of the formula (I),

wherein
X is C═O;
y is 1; and
R is aryl, especially phenyl,
   can be obtained by converting the corresponding compound of the formula (I), wherein R is H with an arylboronic acid, especially phenylboronic acid; in the presence of anhydrous cupric acetate and a tertiary amine, e.g., a tri-lower alkylamine, such as triethylamine; in an appropriate aprotic solvent, especially a halogenated hydrocarbon, such as dichloromethylene; at preferred temperatures between 0° C. and 50° C., e.g., at RT, into said compound.

Salts of compounds of formula (I) having at least one salt-forming group may be prepared in a manner known per se. For example, salts of compounds of formula (I) having acid groups may be formed, e.g., by treating the compounds with metal compounds, such as alkali metal salts of suitable organic carboxylic acids, e.g., the sodium salt of 2-ethylhexanoic acid; with organic alkali metal or alkaline earth metal compounds, such as the corresponding hydroxides, carbonates or hydrogen carbonates, such as sodium or potassium hydroxide, carbonate or hydrogen carbonate; with corresponding calcium compounds or with ammonia or a suitable organic amine, stoichiometric amounts or only a small excess of the salt-forming agent preferably being used. Acid addition salts of compounds of formula (I) are obtained in customary manner, e.g., by treating the compounds with an acid or a suitable anion exchange reagent. Internal salts of compounds of formula (I) containing acid and basic salt-forming groups, e.g., a free carboxy group and a free amino group, may be formed, e.g., by the neutralization of salts, such as acid addition salts, to the isoelectric point, e.g., with weak bases, or by treatment with ion exchangers.

Salts can be converted in customary manner into the free compounds; metal and ammonium salts can be converted, e.g., by treatment with suitable acids; and acid addition salts, e.g., by treatment with a suitable basic agent.

Mixtures of isomers obtainable according to the invention can be separated in a manner known per se into the individual isomers; diastereoisomers can be separated, e.g., by partitioning between polyphasic solvent mixtures, recrystallization and/or chromatographic separation, e.g., over silica gel or by, e.g., medium pressure liquid chromatography over a reversed phase column; and racemates can be separated, e.g., by the formation of salts with optically pure salt-forming reagents and separation of the mixture of diastereoisomers so obtainable, e.g., by means of fractional crystallization, or by chromatography over optically active column materials.

Intermediates and final products can be worked up and/or purified according to standard methods, e.g., using chromatographic methods, distribution methods, re-crystallization and the like.

### Additional Process Steps

In the additional process steps, carried out as desired, functional groups of the starting compounds which should not take part in the reaction may be present in unprotected form or may be protected, e.g., by one or more protecting groups. The protecting groups are then wholly or partly removed according to one of the known methods.

Protecting groups, and the manner in which they are introduced and removed are described, e.g., Protective Groups in Organic Chemistry, Plenum Press, London, N.Y. (1973); Methoden der organischen Chemie, Houben-Weyl, 4^{th} Edition, Vol. 15/1, Georg-Thieme-Verlag, Stuttgart (1974); and Theodora W. Greene, Protective Groups in Organic Synthesis, John Wiley & Sons, NY (1981). A characteristic of protecting groups is that they can be removed readily, i.e., without the occurrence of undesired secondary reactions, e.g., by solvolysis, reduction, photolysis or alternatively under physiological conditions.

The end products of formula (I) may however also contain substituents that can also be used as protecting groups in starting materials for the preparation of other end products of formula (I). Thus, within the scope of this text, only a readily removable group that is not a constituent of the particular desired end product of formula (I) is designated a “protecting group”, unless the context indicates otherwise.

### General Process Conditions

The following applies in general to all processes mentioned hereinbefore and hereinafter, while reaction conditions specifically mentioned above or below are preferred:

All the above-mentioned process steps can be carried out under reaction conditions that are known per se, preferably those mentioned specifically, in the absence or, customarily, in the presence of solvents or diluents, preferably solvents or diluents that are inert towards the reagents used and dissolve them, in the absence or presence of catalysts, condensation or neutralizing agents, e.g., Ion exchangers, such as cation exchangers, e.g., in the H⁺ form; depending on the nature of the reaction and/or of the reactants at reduced, normal or elevated temperature, e.g., in a temperature range of from about −100° C. to about 190° C.; preferably from approximately −80° C. to approximately 150° C., e.g., at from −80° C. to −60° C. at RT, at from −20° C. to 40° C. or at reflux temperature; under atmospheric pressure or in a closed vessel, where appropriate under pressure and/or in an inert atmosphere, e.g., under an argon or nitrogen atmosphere.

At all stages of the reactions, mixtures of isomers that are formed can be separated into the individual isomers, e.g., diastereoisomers or enantiomers; or into any desired mixtures of isomers, e.g., racemates or mixtures of diastereoisomers, e.g., analogously to the methods described under “additional process steps”.

The solvents from which those solvents that are suitable for any particular reaction may be selected include those mentioned specifically or, e.g., water, esters, such as lower alkyl-lower alkanoates, e.g., ethyl acetate; ethers, such as aliphatic ethers, e.g., diethyl ether; or cyclic ethers, e.g., tetrahydrofuran or dioxane; liquid aromatic hydrocarbons, such as benzene or toluene; alcohols, such as methanol, ethanol or 1- or 2-propanol; nitrites, such as acetonitrile; halogenated hydrocarbons, such as methylene chloride or chloroform; acid amides, such as dimethylformamide or dimethyl acetamide; bases, such as heterocyclic nitrogen bases, e.g., pyridine or N-methylpyrrolidin-2-one; carboxylic acid anhydrides, such as lower alkanoic acid anhydrides, e.g., acetic anhydride; cyclic, linear or branched hydrocarbons, such as cyclohexane, hexane or isopentane; or mixtures of those solvents, e.g., aqueous solutions, unless otherwise indicated in the description of the processes. Such solvent mixtures may also be used in working up, e.g., by chromatography or partitioning.

The compounds, including their salts, may also be obtained in the form of hydrates, or their crystals may, e.g., include the solvent used for crystallization. Different crystalline forms may be present.

The invention relates also to those forms of the process in which a compound obtainable as intermediate at any stage of the process is used as starting material and the remaining process steps are carried out, or in which a starting material is formed under the reaction conditions or is used in the form of a derivative, e.g., in protected form or in the form of a salt, or a compound obtainable by the process according to the invention is produced under the process conditions and processed further in situ. In the process of the present invention those starting materials are preferably used which result in new compounds of formula (I) described at the beginning as being especially valuable. Special preference is given to reaction conditions that are analogous to those mentioned in the examples.

### Pharmaceutical Compositions

The invention relates also to pharmaceutical compositions comprising a compound of formula (I), to their use in the therapeutic (in a broader aspect of the invention also prophylactic) treatment or a method of treatment of a protein kinase dependent disease, especially the preferred diseases mentioned above, to the compounds for said use and to the preparation of pharmaceutical preparations, especially for said uses.

The present invention also relates to pro-drugs of a compound of formula (I) that convert in vivo to the compound of formula (I) as such. Any reference to a compound of formula (I) is therefore to be understood as referring also to the corresponding pro-drugs of the compound of formula (I), as appropriate and expedient. The pharmacologically acceptable compounds of the present invention may be used, e.g., for the preparation of pharmaceutical compositions that comprise an effective amount of a compound of the formula (I), or a pharmaceutically acceptable salt thereof, as active ingredient together or in admixture with a significant amount of one or more inorganic or organic, solid or liquid, pharmaceutically acceptable carriers.

The invention relates also to a pharmaceutical composition that is suitable for administration to a warm-blooded animal, especially a human (or to cells or cell lines derived from a warm-blooded animal, especially a human, e.g., lymphocytes), for the treatment or, in a broader aspect of the invention, prevention of (=prophylaxis against) a disease that responds to inhibition of protein kinase activity, comprising an amount of a compound of formula (I) or a pharmaceutically acceptable salt thereof, which is effective for said inhibition, especially the in, together with at least one pharmaceutically acceptable carrier.

The pharmaceutical compositions according to the invention are those for enteral, such as nasal; rectal or oral; or parenteral, such as intramuscular or intravenous, administration to warm-blooded animals (especially a human), that comprise an effective dose of the pharmacologically active ingredient, alone or together with a significant amount of a pharmaceutically acceptable carrier. The dose of the active ingredient depends on the species of warm-blooded animal, the body weight, the age and the individual condition, individual pharmacokinetic data, the disease to be treated and the mode of administration.

The invention relates also to a method of treatment for a disease that responds to inhibition of a protein kinase, which comprises administering an (against the mentioned disease) prophylactically or especially therapeutically effective amount of a compound of formula (I) according to the invention, especially to a warm-blooded animal, e.g., a human, that, on account of one of the mentioned diseases, requires such treatment.

The dose of a compound of the formula (I) or a pharmaceutically acceptable salt thereof to be administered to warm-blooded animals, e.g., humans of approximately 70 kg body weight, is preferably from approximately 3 mg to approximately 10 g, more preferably from approximately 10 mg to approximately 1.5 g, most preferably from about 100 mg to about 1000 mg/person/day, divided preferably into 1-3 single doses which may, e.g., be of the same size. Usually, children receive half of the adult dose.

The pharmaceutical compositions comprise from approximately 1% to approximately 95%, preferably from approximately 20% to approximately 90%, active ingredient. Pharmaceutical compositions according to the invention may be, e.g., in unit dose form, such as in the form of ampoules, vials, suppositories, dragées, tablets or capsules.

The pharmaceutical compositions of the present invention are prepared in a manner known per se, e.g., by means of conventional dissolving, lyophilizing, mixing, granulating or confectioning processes.

Solutions of the active ingredient, and also suspensions, and especially isotonic aqueous solutions or suspensions, are preferably used, it being possible, e.g., in the case of lyophilized compositions that comprise the active ingredient alone or together with a carrier, e.g., mannitol, for such solutions or suspensions to be produced prior to use. The pharmaceutical compositions may be sterilized and/or may comprise excipients, e.g., preservatives, stabilizers, wetting and/or emulsifying agents, solubilizers, salts for regulating the osmotic pressure and/or buffers; and are prepared in a manner known per se, e.g., by means of conventional dissolving or lyophilizing processes. The said solutions or suspensions may comprise viscosity-increasing substances, such as sodium carboxymethylcellulose, carboxymethylcellulose, dextran, polyvinylpyrrolidone or gelatin.

Suspensions in oil comprise as the oil component the vegetable, synthetic or semi-synthetic oils customary for injection purposes. There may be mentioned as such especially liquid fatty acid esters that contain as the acid component a long-chained fatty acid having from 8-22 carbon atoms, especially from 12-22 carbon atoms, e.g., lauric acid, tridecylic acid, myristic acid, pentadecylic acid, palmitic acid, margaric acid, stearic acid, arachidic acid, behenic acid or corresponding unsaturated acids, e.g., oleic acid, elaidic acid, erucic acid, brasidic acid or linoleic acid, if desired with the addition of antioxidants, e.g., vitamin E, β-carotene or 3,5-di-tert-butyl-4-hydroxytoluene. The alcohol component of those fatty acid esters has a maximum of 6 carbon atoms and is a mono- or poly-hydroxy, e.g., a mono-, di- or tri-hydroxy; alcohol, e.g., methanol, ethanol, propanol, butanol or pentanol; or the isomers thereof, but especially glycol and glycerol. The following examples of fatty acid esters are therefore to be mentioned: ethyl oleate, isopropyl myristate, isopropyl palmitate, “Labrafil M 2375” (polyoxyethylene glycerol trioleate, Gattefossé, Paris), “Miglyol 812” (triglyceride of saturated fatty acids with a chain length of C₈-C₁₂, Hüls A G, Germany), but especially vegetable oils, such as cottonseed oil, almond oil, olive oil, castor oil, sesame oil, soybean oil and more especially groundnut oil.

The injection compositions are prepared in customary manner under sterile conditions; the same applies also to introducing the compositions into ampoules or vials and sealing the containers.

Pharmaceutical compositions for oral administration can be obtained by combining the active ingredient with solid carriers, if desired granulating a resulting mixture, and processing the mixture, if desired or necessary, after the addition of appropriate excipients, into tablets, dragée cores or capsules. It is also possible for them to be incorporated into plastics carriers that allow the active ingredients to diffuse or be released in measured amounts. Suitable carriers are especially fillers, such as sugars, e.g., lactose, saccharose, mannitol or sorbitol; cellulose preparations and/or calcium phosphates, e.g., tricalcium phosphate or calcium hydrogen phosphate; and binders, such as starch pastes using, e.g., corn, wheat, rice or potato starch, gelatin, tragacanth, methylcellulose, hydroxypropylmethylcellulose, sodium carboxymethylcellulose and/or polyvinylpyrrolidone; and/or, if desired, disintegrators, such as the above-mentioned starches; and/or carboxymethyl starch, crosslinked polyvinylpyrrolidone, agar, alginic acid or a salt thereof, such as sodium alginate. Excipients are especially flow conditioners and lubricants, e.g., silicic acid, talc, stearic acid or salts thereof, such as magnesium or calcium stearate; and/or polyethylene glycol. Dragée cores are provided with suitable, optionally enteric, coatings, there being used, inter alia, concentrated sugar solutions which may comprise gum arabic, talc, polyvinylpyrrolidone, polyethylene glycol and/or titanium dioxide; or coating solutions in suitable organic solvents, or, for the preparation of enteric coatings, solutions of suitable cellulose preparations, such as ethylcellulose phthalate or hydroxypropylmethylcellulose phthalate. Capsules are dry-filled capsules made of gelatin and soft sealed capsules made of gelatin and a plasticizer, such as glycerol or sorbitol. The dry-filled capsules may comprise the active ingredient in the form of granules, e.g., with fillers, such as lactose; binders, such as starches; and/or glidants, such as talc or magnesium stearate; and if desired with stabilizers. In soft capsules the active ingredient is preferably dissolved or suspended in suitable oily excipients, such as fatty oils, paraffin oil or liquid polyethylene glycols, it being possible also for stabilizers and/or antibacterial agents to be added. Dyes or pigments may be added to the tablets or dragée coatings or the capsule casings, e.g., for identification purposes or to indicate different doses of active ingredient.

### Combinations

A compound of the formula (I) may also be used to advantage in combination with other antiproliferative agents. Such antiproliferative agents include, but are not limited to, aromatase inhibitors; antiestrogens; topoisomerase I inhibitors; topoisomerase II inhibitors; microtubule active agents; alkylating agents; histone deacetylase inhibitors; compounds, which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; bisphosphonates; biological response modifiers; antiproliferative antibodies; heparanase inhibitors; inhibitors of Ras oncogenic isoforms; telomerase inhibitors; proteasome inhibitors; agents used in the treatment of hematologic malignancies; compounds which target, decrease or inhibit the activity of Flt-3; Hsp90 inhibitors; temozolomide (TEMODAL®); and leucovorin.

The term “aromatase inhibitor”, as used herein, relates to a compound which inhibits the estrogen production, i.e., the conversion of the substrates androstenedione and testosterone to estrone and estradiol, respectively. The term includes, but is not limited to, steroids, especially atamestane, exemestane and formestane; and, in particular, non-steroids, especially aminoglutethimide, roglethimide, pyridoglutethimide, trilostane, testolactone, ketokonazole, vorozole, fadrozole, anastrozole and letrozole. Exemestane can be administered, e.g., in the form as it is marketed, e.g., under the trademark AROMASIN. Formestane can be administered, e.g., in the form as it is marketed, e.g., under the trademark LENTARON. Fadrozole can be administered, e.g., in the form as it is marketed, e.g., under the trademark AFEMA. Anastrozole can be administered, e.g., in the form as it is marketed, e.g., under the trademark ARIMIDEX. Letrozole can be administered, e.g., in the form as it is marketed, e.g., under the trademark FEMARA or FEMAR. Aminoglutethimide can be administered, e.g., in the form as it is marketed, e.g., under the trademark ORIMETEN. A combination of the invention comprising a chemotherapeutic agent which is an aromatase inhibitor is particularly useful for the treatment of hormone receptor positive tumors, e.g., breast tumors.

The term “ant-estrogen”, as used herein, relates to a compound which antagonizes the effect of estrogens at the estrogen receptor level. The term includes, but is not limited to, tamoxifen, fulvestrant, raloxifene and raloxifene hydrochloride. Tamoxifen can be administered, e.g., in the form as it is marketed, e.g., under the trademark NOLVADEX. Raloxifene hydrochloride can be administered, e.g., in the form as it is marketed, e.g., under the trademark EVISTA. Fulvestrant can be formulated as disclosed in U.S. Pat. No. 4,659,516 or it can be administered, e.g., in the form as it is marketed, e.g., under the trademark FASLODEX. A combination of the invention comprising a chemotherapeutic agent which is an antiestrogen is particularly useful for the treatment of estrogen receptor positive tumors, e.g., breast tumors.

The term “anti-androgen”, as used herein, relates to any substance which is capable of inhibiting the biological effects of androgenic hormones and Includes, but is not limited to, bicalutamide (CASODEX), which can be formulated, e.g., as disclosed in U.S. Pat. No. 4,636,505.

The term “gonadorelin agonist”, as used herein, includes, but is not limited to, abarelix, goserelin and goserelin acetate. Goserelin is disclosed in U.S. Pat. No. 4,100,274 and can be administered, e.g., in the form as it is marketed, e.g., under the trademark ZOLADEX. Abarelix can be formulated, e.g., as disclosed in U.S. Pat. No. 5,843,901.

The term “topoisomerase I inhibitor”, as used herein, includes, but is not limited to, topotecan, gimatecan, irinotecan, camptothecian and its analogues, 9-nitrocamptothecin and the macromolecular camptothecin conjugate PNU-166148 (compound A1 in WO 99/17804). Irinotecan can be administered, e.g., in the form as it is marketed, e.g., under the trademark CAMPTOSAR. Topotecan can be administered, e.g., in the form as it is marketed, e.g., under the trademark HYCAMTIN.

The term “topoisomerase II inhibitor”, as used herein, includes, but is not limited to, the anthracyclines, such as doxorubicin, including liposomal formulation, e.g., CAELYX; daunorubicin; epirubicin; idarubicin; nemorubicin; the anthraquinones mitoxantrone and losoxantrone; and the podophillotoxines etoposide and teniposide. Etoposide can be administered, e.g., in the form as it is marketed, e.g., under the trademark ETOPOPHOS. Teniposide can be administered, e.g., in the form as it is marketed, e.g., under the trademark VM 26-BRISTOL. Doxorubicin can be administered, e.g., in the form as it is marketed, e.g., under the trademark ADRIBLASTIN or ADRIAMYCIN. Epirubicin can be administered, e.g., in the form as it is marketed, e.g., under the trademark FARMORUBICIN. Idarubicin can be administered, e.g., in the form as it is marketed, e.g., under the trademark ZAVEDOS. Mitoxantrone can be administered, e.g., in the form as it is marketed, e.g., under the trademark NOVANTRON.

The term “microtubule active agent” relates to microtubule stabilizing, microtubule destabilizing agents and microtublin polymerization inhibitors including, but not limited to, taxanes, e.g., paclitaxel and docetaxel; vinca alkaloids, e.g., vinblastine, especially vinblastine sulfate; vincristine, especially vincristine sulfate and vinorelbine; discodermolides; cochicine; and epothilones and derivatives thereof, e.g., epothilone B or D or derivatives thereof. Paclitaxel may be administered, e.g., in the form as it is marketed, e.g., TAXOL. Docetaxel can be administered, e.g., in the form as it is marketed, e.g., under the trademark TAXOTERE. Vinblastine sulfate can be administered, e.g., in the form as it is marketed, e.g., under the trademark VINBLASTIN R.P. Vincristine sulfate can be administered, e.g., in the form as it is marketed, e.g., under the trademark FARMISTIN. Discodermolide can be obtained, e.g., as disclosed in U.S. Pat. No. 5,010,099. Also included are epothilone derivatives which are disclosed in WO 98/10121, U.S. Pat. No. 6,194,181, WO 98/25929, WO 98/08849, WO 99/43653, WO 98/22461 and WO 00/31247. Especially preferred are epothilone A and/or B.

The term “alkylating agent”, as used herein, includes, but is not limited to, cyclophosphamide, ifosfamide, melphalan or nitrosourea (BCNU or Gliadel). Cyclophosphamide can be administered, e.g., in the form as it is marketed, e.g., under the trademark CYCLOSTIN. Ifosfamide can be administered, e.g., in the form as it is marketed, e.g., under the trademark HOLOXAN.

The term “histone deacetylase inhibitors” or “HDAC inhibitors” relates to compounds which inhibit the histone deacetylase and which possess antiproliferative activity. This includes compounds disclosed in WO 02/22577, especially N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(2-methyl-1H-indol-3-yl)-ethyl]-amino]methyl]phenyl]-2E-2-propenamide and pharmaceutically acceptable salts thereof. It further especially includes suberoylanilide hydroxamic acid (SAHA).

The term “antineoplastic antimetabolite” includes, but is not limited to, 5-fluorouracil or 5-FU; capecitabine; gemcitabine; DNA demethylating agents, such as 5-azacytidine and decitabine; methotrexate and edatrexate; and folic acid antagonists, such as pemetrexed. Capecitabine can be administered, e.g., in the form as it is marketed, e.g., under the trademark XELODA. Gemcitabine can be administered, e.g., in the form as it is marketed, e.g., under the trademark GEMZAR. Also included is the monoclonal antibody trastuzumab which can be administered, e.g., in the form as it is marketed, e.g., under the trademark HERCEPTIN.

The term “platin compound”, as used herein, includes, but is not limited to, carboplatin, cis-platin, cisplatinum and oxaliplatin. Carboplatin can be administered, e.g., in the form as it is marketed, e.g., under the trademark CARBOPLAT. Oxaliplatin can be administered, e.g., in the form as it is marketed, e.g., under the trademark ELOXATIN.

The term “compounds targeting/decreasing a protein or lipid kinase activity; or a protein or lipid phosphatase activity; or further anti-angiogenic compounds”, as used herein, includes, but is not limited to, protein tyrosine kinase and/or serine and/or threonine kinase inhibitors or lipid kinase inhibitors, e.g.,
a) compounds targeting, decreasing or Inhibiting the activity of the platelet-derived growth factor-receptors (PDGFR), such as compounds which target, decrease or inhibit the activity of PDGFR, especially compounds which inhibit the PDGF receptor, e.g., a N-phenyl-2-pyrimidine-amine derivative, e.g., imatinib, SU101, SU6668 and GFB-111;
b) compounds targeting, decreasing or inhibiting the activity of the fibroblast growth factor-receptors (FGFR);
c) compounds targeting, decreasing or inhibiting the activity of the insulin-like growth factor receptor I (IGF-IR), such as compounds which target, decrease or inhibit the activity of IGF-IR, especially compounds which inhibit the IGF-IR receptor, such as those compounds disclosed in WO 02/092599;
d) compounds targeting, decreasing or inhibiting the activity of the Trk receptor tyrosine kinase family;
e) compounds targeting, decreasing or Inhibiting the activity of the Axl receptor tyrosine kinase family;
f) compounds targeting, decreasing or inhibiting the activity of the c-Met receptor;
g) compounds targeting, decreasing or inhibiting the activity of the Kit/SCFR receptor tyrosine kinase;
h) compounds targeting, decreasing or inhibiting the activity of the C-kit receptor tyrosine kinases—(part of the PDGFR family), such as compounds which target, decrease or inhibit the activity of the c-Kit receptor tyrosine kinase family, especially compounds which inhibit the c-Kit receptor, e.g., imatinib;
i) compounds targeting, decreasing or inhibiting the activity of members of the c-Abl family and their gene-fusion products, e.g., BCR-Abl kinase, such as compounds which target decrease or inhibit the activity of c-Abl family members and their gene fusion products, e.g., a N-phenyl-2-pyrimidine-amine derivative, e.g., imatinib, PD180970, AG957, NSC 680410 or PD173955 from ParkeDavis;
j) compounds targeting, decreasing or inhibiting the activity of members of the protein kinase C (PKC) and Raf family of serine/threonine kinases, members of the MEK, SRC, JAK, FAK, PDK and Ras/MAPK family members, or PI(3) kinase family, or of the PI(3)-kinase-related kinase family, and/or members of the cyclin-dependent kinase family (CDK) and are especially those staurosporine derivatives disclosed in U.S. Pat. No. 5,093,330, e.g., midostaurin; examples of further compounds include, e.g., UCN-01; safingol; BAY 43-9006; Bryostatin 1; Perifosine; Ilmofosine; RO 318220 and RO 320432; GO 6976; Isis 3521; LY333531/LY379196; isochinoline compounds, such as those disclosed in WO 00/09495; FTIs; PD184352; or QAN697 (a P13K inhibitor);
k) compounds targeting, decreasing or inhibiting the activity of protein-tyrosine kinase inhibitors, such as compounds which target, decrease or inhibit the activity of protein-tyrosine kinase inhibitors include imatinib mesylate (GLEEVEC) or tyrphostin. A tyrphostin is preferably a low molecular weight (Mr <1500) compound, or a pharmaceutically acceptable salt thereof, especially a compound selected from the benzylidenemalonitrile class or the S-arylbenzenemalonirile or bisubstrate quinoline class of compounds, more especially any compound selected from the group consisting of Tyrphostin A23/RG-50810, AG 99, Tyrphostin AG 213, Tyrphostin AG 1748, Tyrphostin AG 490, Tyrphostin B44, Tyrphostin B44 (+) enantiomer, Tyrphostin AG 555, AG 494, Tyrphostin AG 556, AG957 and adaphostin (4-{[(2,5-dihydroxyphenyl)methyl]amino}-benzoic acid adamantyl ester, NSC 680410, adaphostin; and
l) compounds targeting, decreasing or Inhibiting the activity of the epidermal growth factor family of receptor tyrosine kinases (EGFR, ErbB2, ErbB3, ErbB4 as homo- or hetero-dimers), such as compounds which target, decrease or inhibit the activity of the epidermal growth factor receptor family are especially compounds, proteins or antibodies which inhibit members of the EGF receptor tyrosine kinase family, e.g., EGF receptor, ErbB2, ErbB3 and ErbB4 or bind to EGF or EGF related ligands, and are in particular those compounds, proteins or monoclonal antibodies generically and specifically disclosed in WO 97/02266, e.g., the compound of Example 39, or in EP 0 564 409; WO 99/03854; EP 0520722; EP 0 566 226; EP 0 787 722; EP 0 837 063; U.S. Pat. No. 5,747,498; WO 98/10767; WO 97/30034; WO 97/49688; WO 97/38983 and, especially, WO 96/30347, e.g., compound known as CP 358774; WO 96/33980, e.g., compound ZD 1839; and WO 95/03283, e.g., compound ZM105180, e.g., trastuzumab (HERCEPTIN), cetuximab, Iressa, Tarceva, OSI-774, CI-1033, EKB-569, GW-2016, E1.1, E2.4, E2.5, E6.2, E6.4, E2.11, E6.3 or E7.6.3; and 7H-pyrrolo-[2,3-o]pyrimidine derivatives which are disclosed in WO 03/013541.

Further anti-angiogenic compounds include compounds having another mechanism for their activity, e.g., unrelated to protein or lipid kinase inhibition, e.g., thalidomide (THALOMID) and TNP-470.

Compounds which target, decrease or inhibit the activity of a protein or lipid phosphatase are, e.g., inhibitors of phosphatase 1, phosphatase 2A, PTEN or CDC25, e.g., okadaic acid or a derivative thereof.

Compounds which induce cell differentiation processes are e.g. retinoic acid, α- γ- or δ-tocopherol or α- γ- or δ-tocotrienol.

The term cyclooxygenase inhibitor, as used herein, includes, but is not limited to, e.g., Cox-2 inhibitors, 5-alkyl substituted 2-arylaminophenylacetic acid and derivatives, such as celecoxib (CELEBREX), rofecoxib (VIOXX), etoricoxib, valdecoxib or a 5-alkyl-2-arylaminophenylacetic acid, e.g., 5-methyl-2-(2′-chloro-6′-fluoroanilino)phenyl acetic acid or lumiracoxib.

The term “bisphosphonates”, as used herein, includes, but is not limited to, etridonic, clodronic, tiludronic, pamidronic, alendronic, ibandronic, risedronic and zoledronic acid. “Etridonic acid” can be administered, e.g., in the form as it is marketed, e.g., under the trademark DIDRONEL. “Clodronic acid” can be administered, e.g., in the form as it is marketed, e.g., under the trademark BONEFOS. “Tiludronic acid” can be administered, e.g., in the form as it is marketed, e.g., under the trademark SKELID. “Pamidronic acid” can be administered, e.g., in the form as it is marketed, e.g., under the trademark AREDIA™. “Alendronic acid” can be administered, e.g., in the form as it is marketed, e.g., under the trademark FOSAMAX. “Ibandronic acid” can be administered, e.g., in the form as it is marketed, e.g., under the trademark BONDRANAT. “Risedronic acid” can be administered, e.g., in the form as it is marketed, e.g., under the trademark ACTONEL. “Zoledronic acid” can be administered, e.g., in the form as it is marketed, e.g., under the trademark ZOMETA.

The term “mTOR inhibitors” relates to compounds which inhibit the mammalian target of rapamycin (mTOR) and which possess antiproliferative activity, such as sirolimus (Rapamune®), everolimus (Certican™), CCl-779 and ABT578.

The term “heparanase inhibitor”, as used herein, refers to compounds which target, decrease or inhibit heparin sulphate degradation. The term includes, but is not limited to, PI-88.

The term “biological response modifier”, as used herein, refers to a lymphokine or interferons, e.g., interferon γ.

The term “inhibitor of Ras oncogenic isoforms”, e.g., H-Ras, K-Ras or N-Ras, as used herein, refers to compounds which target, decrease or inhibit the oncogenic activity of Ras, e.g., a “farnesyl transferase inhibitor”, e.g., L-744832, DK8G557 or R115777 (Zamestra).

The term “telomerase inhibitor”, as used herein, refers to compounds which target, decrease or inhibit the activity of telomerase. Compounds which target, decrease or inhibit the activity of telomerase are especially compounds which inhibit the telornerase receptor, e.g., telomestatin.

The term “methionine aminopeptidase inhibitor”, as used herein, refers to compounds which target, decrease or inhibit the activity of methionine aminopeptidase. Compounds which target, decrease or inhibit the activity of methionine aminopeptidase are, e.g., bengamide or a derivative thereof.

The term “proteasome inhibitor”, as used herein, refers to compounds which target, decrease or inhibit the activity of the proteasome. Compounds which target, decrease or inhibit the activity of the proteasome include, e.g., PS-341 and MLN 341

The term “matrix metalloproteinase inhibitor” or “MMP inhibitor”, as used herein, includes, but is not limited to, collagen peptidomimetic and nonpeptidomimetic inhibitors, tetracycline derivatives, e.g., hydroxamate peptidomimefc inhibitor batimastat and its orally bioavailable analogue marimastat (BB-2516), prinomastat (AG3340), metastat (NSC 683551) BMS-279251, BAY 12-9566, TAA211, MMI270B or MJ996.

The term “agents used in the treatment of hematologic malignancies”, as used herein, Includes, but is not limited to, FMS-like tyrosine kinase inhibitors, e.g., compounds targeting, decreasing or inhibiting the activity of FMS-like tyrosine kinase receptors (Flt-3R); interferon, 1-b-D-arabinofuransylcytosine (ara-c) and bisulfan; and ALK inhibitors, e.g., compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, e.g., PKC412, midostaurin, a staurosporine derivative, SU11248 and MLN518.

The term “HSP90 inhibitors”, as used herein, includes, but is not limited to, compounds targeting, decreasing or inhibiting the intrinsic ATPase activity of HSP90; degrading, targeting, decreasing or inhibiting the HSP90 client proteins via the ubiquitin proteasome pathway. Compounds targeting, decreasing or inhibiting the intrinsic ATPase activity of HSP90 are especially compounds, proteins or antibodies which inhibit the ATPase activity of HSP90, e.g., 17-allylamino, 17-demethoxygeldanamycin (17AAG), a geldanamycin derivative, other geldanamycin related compounds, radicicol and HDAC inhibitors.

The term “antiproliferative antibodies”, as used herein, includes, but is not limited to, trastuzumab (Herceptin™), Trastuzumab-DM1, erlotinib (Tarceva™), bevacizumab (Avastin™), rituximab (Rituxan®), PRO64553 (anti-CD40) and 2C4 antibody. By antibodies is meant, e.g., intact monoclonal antibodies, polyclonal antibodies, multispecific antibodies formed from at least two intact antibodies, and antibodies fragments so long as they exhibit the desired biological activity.

For the treatment of acute myeloid leukemia (AML), compounds of formula (I) can be used in combination with standard leukemia therapies, especially in combination with therapies used for the treatment of AML. In particular, compounds of formula (I) can be administered in combination with, e.g., farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

The structure of the active agents identified by code nos., generic or trade names may be taken from the actual edition of the standard compendium “The Merck Index” or from databases, e.g., Patents International, e.g., IMS World Publications.

The above-mentioned compounds, which can be used in combination with a compound of the formula (I), can be prepared and administered as described in the art, such as in the documents cited above.

A compound of the formula (I) may also be used to advantage in combination with known therapeutic processes, e.g., the administration of hormones or especially radiation.

A compound of formula (I) may in particular be used as a radiosensitizer, especially for the treatment of tumors which exhibit poor sensitivity to radiotherapy.

By “combination”, there is meant either a fixed combination in one dosage unit form, or a kit of parts for the combined administration where a compound of the formula (I) and a combination partner may be administered independently at the same time or separately within time intervals that especially allow that the combination partners show a cooperative, e.g., synergistic, effect or any combination thereof.

The following examples are merely illustrative and not meant to limit the scope of the present claims in any manner.

### EXAMPLES

The following examples serve to illustrate the invention without limiting the scope thereof:

Abbreviations

| | |
|---|---|
| Boc | tert-butoxycarbonyl |
| DCM | dichloromethane |
| DMF | N,N-dimethylformamide |
| DMSO | dimethylsulfoxide |
| ES-MS | electrospray mass spectrometry |
| Grad | gradient |
| HCl | hydrochloric acid |
| HPLC | high-pressure liquid |
| | chromatography |
| mL | mililiter(s) |
| NMR | nuclear magnetic resonance |
| PS | polystyrene |
| RT | room temperature |
| t_{R} | HPLC retention time in minutes |
| TFA | trifluoroacetic acid |
| THF | tetrahydrofuran |

Where no temperatures are given, the reaction takes place at ambient (room) temperature. Ratios of solvents, e.g., in eluents or solvent mixtures, are given in volume by volume (v/v). The following agents were obtained from Fluka, Buchs, Switzerland: 4-amino-benzonitrile; 2-amino-5-bromo-benzoic acid; nitromethane; ethyl cyanoacetate; (R)-2-tert-butoxycarbonylamino-3-(4-nitro-phenyl)-propionic acid; N-chlorosuccinimide; 4-nitrophenethyl bromide; triethyl orthoacetate; triethyorthoacetate; 2-aminofluorobenzoic acid; 2-amino-chlorobenzoic acid; cyanogen bromide; 3-formylphenylboronic acid; aniline 4-fluoroaniline; and phenylboronic acid.

The following agents were obtained from Aldrich, Buchs, Switzerland: 3,4-methylenedioxyphenylboronic acid; 3,4-difluoro-1-nitrobenzene; 2-fluoro-5-nitrotoluene; thiophene-2-boronic acid; thiophene-3-boronic acid; benzo[b]furan-2-boronic acid; 2-fluoroaniline; 2,4-dimethoxyphenylboronic acid; 2,5-dimethoxyphenylboronic acid; 3,4-dimethoxyphenylboronic acid; phenylboronic acid; 2,3-dimethoxyphenylboronic acid; 2,3,4-trimethoxyphenylboronic acid; 3-methoxyphenylboronic acid; 3-fluorophenylboronic acid; and (4-amino-phenyl)-acetonitrile.

The following agents were obtained from Lancaster, Morecambe, U.K: benzo[b]thiophene-2-boronic acid; 4-hydroxyphenylboronic acid; 3,4,5-trimethoxyphenylboronic acid; and 4-hydroxyphenylboronic acid.

5-Indolylboronic acid was obtained from Frontier Scientific, inc., Lancashire, U.K.; pyridine-4-boronic acid was obtained from Maybridge, Cornwall, U.K.; and pyridine-3-boronic acid was obtained from Acros, Morris Plains, N.J., USA.

HPLC linear gradient between A=H₂O/TFA 1000:1 and B=acetonitrile/TFA 1000:1.

Grad 1: 2-100% B in 7 minutes and 3 minutes at 100% B; column: Nucleosil C₁₈ reverse phase; 250 mm×4.6 mm; particle size 5 μm, 100 Å; flow rate: 2.0 mL/min.; detection at 215 nm.

Grad 2: 20-100% B in 5 minutes and 1.5 minutes at 100% B; column: Nucleosil C₁₈ reverse phase; 70 mm×4 mm; particle size 3 μm, 100 Å; flow rate: 1.25 mL/min.; detection at 215 nm.

Grad 3: 2-100% B in 4.5 minutes and 1 minute at 100% B; column: Chromolith Performance; 100 mm×4.5 mm; flow rate: 2 mL/min.; detection at 215 nm.

Grad 4: 12-70% in 2.5 minutes; column: Chromolith SpeedROD RP18e; 500 mm×4.6 mm; flow rate: 4 mL/min.; detection at 210 nm.

Grad 5: 20-100% B in 5 minutes and 1 minute at 100% B; column: Nucleosil C₁₈ reverse phase; 250 mm×4.6 mm; particle size 5 μm, 100 Å; flow rate: 1.0 mL/min.; detection at 215 nm.

Grad 6: 20-100% B in 14 minutes and 5 minutes at 100%; column: Nucleosil C₁₈ reverse phase; 250 mm×4.6 mm; particle size 5 μm, 100 Å; flow rate: 1.0 mL/min., detection at 215 nm.

Grad 7: 20-100% B in 2.5 minutes; column: Chromolith SpeedROD RP18e; 500 mm×4.6 mm; flow rate: 4 mL/min.; detection at 210 nm.

### Example 1

### [4-(8-Benzo[1,3]dioxol-5-yl-imidazo[4,5-c]quinolin-1-yl)-phenyl]-acetonitrile

76 mg (0.45 mmol) of 3,4-methylenedioxyphenylboronic acid, 10 mg of bis(triphenylphosphine)palladium (II) dichloride and 0.75 mL of a 1 M solution of sodium carbonate are added to a solution of 109 mg (0.30 mmol) of [4-(8-bromo-imidazo[4,5-c]quinolin-1-yl)-phenyl]-acetonitrile (Example 1f) in 3 mL of DMF. The mixture is stirred for 1 hour at 100° C. After filtration, the solution is purified by medium-pressure liquid chromatography to provide [4-(8-benzo[1,3]dioxol-5-yl-imidazo[4,5-c]quinolin-1-yl)-phenyl]-acetonitrile, analytical HPLC: t_{R}=7.19 minutes (Grad 1); ES⁺-MS: m/eₒ=405.1.

### Example 1a

### 5-Bromo-2-(2-nitro-vinylamino)-benzoic acid

A suspension of 25 g (16 mmol) of 2-amino-5-bromo-benzoic acid in H₂O:HCl (37%) (10:1) is stirred for 8 hours and then filtered (Solution A). 8.17 g (255 mmol) of nitromethane are added over 10 minutes to an ice-bath cooled mixture of 35 g of ice and 15.3 g (382 mmol) of NaOH. After stirring for 1 hour at 0° C. and 1 hour at RT, the solution is added at 0° C. to 28 g of ice and 42 mL of HCl (37%) (Solution B). Solutions A and B are combined and the reaction mixture is stirred for 18 hours at RT. The yellow precipitate is filtered-off and washed with water. 5-Bromo-2-(2-nitro-vinylamino)-benzoic acid is dried in vacuo at 40° C., analytical HPLC: t_{R}=3.93 minutes (Grad 1); ES⁺-MS: m/eₒ=287.0, 289.0, Br pattern.

¹H NMR (DMSO-d₆): δ 13.7-14.6 (br, s, 1H), 12.94 (d, 1H), 8.07 (d, 1H), 8.03 (dd, 1H), 7.83 (dd, 1H), 7.71 (d, 1H), 6.76 (d, 1H).

### Example 1b

### 6-Bromo-3-nitro-quinolin-4-ol

29 g (101 mmol) of 5-bromo-2-(2-nitro-vinylamino)-benzoic acid (Example 1a) and 11.9 g (121 mmol) of potassium acetate in 129 mL (152 mmol) of acetic anhydride are stirred for 1.5 hours at 120° C. The precipitate is filtered-off and washed with acetic acid until the filtrate is colorless and then with water. 6-Bromo-3-nitro-quinolin-4-ol is dried in vacuo, analytical HPLC: t_{R}=3.01 minutes (Grad 1); ES⁺-MS: m/eₒ=269.0, 271.0.

### Example 1c

### 6-Bromo-4-chloro-3-nitro-quinoline

7.8 g (29 mmol) of 6-bromo-3-nitro-quinolin-4-ol (Example 1b) in 58 mL (230 mmol) of PO Cl₃ are stirred for 2 hours at 120° C. The mixture is cooled to RT and poured slowly into ice-water. The precipitate is filtered-off, washed with ice-cold water and dissolved in CH₂Cl₂. The organic phase is washed with cold brine, and the aqueous phase is discarded. After drying over MgSO₄, the organic solvent is evaporated to dryness to provide 6-bromo-4-chloro-3-nitro-quinoline, analytical HPLC: t_{R}=4.32 minutes (Grad 1).

¹H NMR (CDCl₃): δ 9.20 (s, 1H), 8.54 (d, 1H), 8.04 (d, 1H), 7.96 (dd, 1H).

### Example 1d

### [4-(6-Bromo-3-nitro-quinolin ylamino)-phenyl]-acetonitrile

0.38 g (3.11 mmol) of 4-amino-benzonitrile are added to a stirred solution of 0.8 g (2.78 mmol) of 6-bromo-4-chloro-3-nitro-quinoline (Example 1c) in 20 mL of acetic acid. The solution is stirred for 1 hour at RT, and after this time, 600 mL of water are added. The precipitate is filtered-off, washed with water and dried overnight to provide the [4-(6-bromo-3-nitro-quinolin-4-ylamino)-phenyl]-acetonitrile, analytical HPLC: t_{R}=8.25 minutes (Grad 1); ES⁺-MS: m/eₒ=369.2.

### Example 1e

### [4-(3-Amino-6-bromo-quinoline-4-ylamino)-phenyl]-acetonitrile

1 g (2.7 mmol) of [4-(6-bromo-3-nitro-quinolinylamino)-phenyl]-acetonitrile are dissolved in 30 mL of MeOH:THF (1:1) and hydrogenated at RT in the presence of 0.5 g of Ni-Raney. The catalyst is filtered-off and washed with methanol. The solvent is evaporated to dryness to provide [4-(3-amino-6-bromo-quinoline-4-ylamino)-phenyl]-acetonitrile, analytical HPLC: t_{R}=6.99 minutes (Grad 1); ES⁺-MS: m/eₒ=339.1, 341.1.

### Example 1f

### [4-(8-Bromo-imidazo[4,5-c]quinolin-1-yl)-phenyl]-acetonitrile

1.0 g (3.1 mmol) of 4-(3-amino-6-bromo-quinoline-4-ylamino-phenyl)-acetonitrile (Example 1e) in 60 mL of triethyl orthoformate are heated at reflux for 2 hours. The reaction mixture is cooled at RT. The precipitate is collected by filtration and purified by medium-pressure liquid chromatography to provide [4-(8-bromo-imidazo[4,5-c]quinolin-1-yl)-phenyl]-acetonitrile, analytical HPLC: t_{R}=6.72 minutes (Grad 1); ES⁺-MS: m/eₒ=349.1, 351.1.

### Example 2

### [4-(8-Thiophen-2-yl-imidazo[4,5-c]quinolin-1-yl)-phenyl]-acetonitrile

[4-(8-Thiophen-2-yl-imidazo[4,5-c]quinolin-1-yl)-phenyl]-acetonitrile is synthesized as described in Example 1 using thiophene-2-boronoc acid. [4-(8-Thiophen-2-yl-imidazo[4,5-c]quinolin-1-yl)phenyl]-acetonitrile, analytical HPLC: t_{R}=7.36 minutes (Grad 1); ES⁺-MS: m/eₒ=367.0.

### Example 3

### [4-(8-Benzofuran-2-yl-imidazo[4,5-c]quinolin-1-yl)-phenyl]-acetonitrile

[4-(8-Benzofuran-2-yl-imidazo[4,5-c]quinolin-1-yl)-phenyl]-acetonitrile is synthesized as described in Example 1 using benzo[b]furan-2-boronic acid. [4-(8-Benzofuran-2-yl-imidazo[4,5-c]quinolin-1-yl)-phenyl]-acetonitrile, analytical HPLC: t_{R}=8.34 minutes (Grad 1); ES⁺-MS: m/eₒ=401.1.

### Example 4

### 2-[4-(8-Benzo[1,3]dioxol-5-yl-Imidazo[4,5-c]quinolin-1-yl)-phenyl]-ethylamine

30 mg (0.074 mmol) of [4-(8-benzo[1,3]dioxol-5-yl-imidazo[4,5-c]quinolin-1-yl)-phenyl]-acetonitrile (Example 1) in 4-mL of 10% NH₃ in methanol:THF (1:1) are hydrogenated at 40° C. in the presence of 10 mg of Ni-Raney. The catalyst is filtered-off and washed with ethyl acetate. The organic solution is washed with water, dried over MgSO₄ and concentrated to dryness to provide 2-[4-(8-benzo[1,3]dioxol-5-yl-imidazo[4,5-c]quinolin-1-yl)-phenyl]-ethylamine, analytical HPLC: t_{R}=6.12 minutes (Grad 3); ES⁺-MS: m/eₒ=409.1.

### Example 5

### 2-[4-(8-Thiophen-2-yl-imidazo[4,5-c]quinolin-1-yl)-phenyl]-ethylamine

2-[4-(8-Thiophen-2-yl-imidazo[4,5-c]quinolin-1-yl)-phenyl]-ethylamine is obtained as described in Example 4 using [4-(8-thiophen-2-yl-imidazo[4,5c]quinolin-1-yl)-phenyl]-acetonitrile (Example 2) as starting material. 2-[4-(8-Thiophen-2-yl-imidazo[4,5-c]quinolin-1-yl)-phenyl]-ethylamine, analytical HPLC: t_{R}=6.35 minutes (Grad 1); ES⁺-MS: m/eₒ=371.3.

### Example 6

### 2-[4-(8-Benzofuran-2-yl-imidazo[4,5-c]quinolin-1-yl)-phenyl]-ethylamine

2-[4-(8-Benzofuran-2-yl-imidazo[4,5-c]quinolin-1-yl)-phenyl]-ethylamine is obtained as described in Example 4 using [4-(8-benzofuran-2-yl-imidazo[4,5-c]quinolin-1-yl)-phenyl]-acetonitrile (Example 3) as starting material. 2-[4-(8-Benzofuran-2-yl-imidazo[4,5-c]quinolin-1-yl)-phenyl]-ethylamine, analytical HPLC: t_{R}=6.95 minutes (Grad 1); ES⁺-MS: m/eₒ=405.2.

### Example 7

### [3-Chloro-4-(8-thiophen-2-yl-imidazo[4,5-c]quinolin-1-yl)-phenyl]-acetonitrile

[3-Chloro-4-(8-thiophen-2-yl-imidazo[4,5-c]quinolin-1-yl)-phenyl]-acetonitrile is synthesized as described in Example 1 using (4-amino-3-chloro-phenyl)-acetonitrile (Example 7a) and thiophene-2-boronoc acid. 3-Chloro-4-(8-thiophen-2-yl-imidazo[4,5-c]quinolin-1-yl)-phenyl]-acetonitrile, analytical HPLC: t_{R}=6.74 minutes (Grad 1); ES⁺-MS: m/eₒ=401.0.

### Example 7a

### (4-Amino-3-chloro-phenyl)-acetonitrile

2.86 g (21 mmol) of N-chlorosuccinimide are added to a stirred solution of 2.67 g (20 mmol) of 4-amino-benzonitrile in 30 mL of Isopropanol. The solution is refluxed for 1 hour and then the solvent is removed in vacuo. The crude product is dissolved in ethyl acetate and water. The layers are separated and the organic layer is washed with brine, dried over MgSO₄ and concentrated in vacuo. The crude residue is purified by chromatography on silica eluting with DCM to afford (4-amino-3-chloro-phenyl)-acetonitrile, analytical HPLC: t_{R}=6.54 minutes (Grad 1); ES⁺-MS: me/eₒ=167.2.

### Example 8

### {3-Chloro-4-[8-(1H-indol-5-yl)-imidazo[4,5-c]quinolin-1-yl]-phenyl}-acetonitrile

{3-Chloro-4-[8-(1H-indol-5-yl)-imidazo[4,5-c]quinolin-1-yl]-phenyl}-acetonitrile is synthesized as described in Example 1 using (4-amino-3-chloro-phenyl)-acetonitrile (Example 7a) and 5-indolylboronic acid. {3-Chloro-4-[8-(1H-indol-5-yl)-imidazo[4,5-c]quinolin-1-yl]-phenyl}acetonitrile, analytical HPLC: t_{R}=6.65 minutes (Grad 1); ES⁺-MS: m/eₒ=434.1.

### Example 9

### [3-Chloro-4-(8-thiophen-3-yl-imidazo[4,5-c]quinolin-1-yl)-phenyl]-acetonitrile

[3-Chloro-4-(8-thiophen-3-yl-imidazo[4,5-c]quinolin-1-yl)-phenyl]-acetonitrile is synthesized as described in Example 1 using (4-amino-3-chloro-phenyl)-acetonitrile (Example 7a) and thiophene-3-boronic acid. [3-Chloro-4-(8-thiophen-3-yl-imidazo[4,5-c]quinolin-1-yl)-phenyl]-acetonitrile, analytical HPLC: t_{R}=6.69 minutes (Grad 1); ES⁺-MS: m/eₒ=401.0.

### Example 10

### 2-[3-Chloro-4-(8-thiophen-2-yl-imidazo[4,5-c]quinolin-1-yl)-phenyl]-ethylamine

2-[3-Chloro-4-(8-thiophen-2-yl-imidazo[4,5-c]quinolin-1-yl)-phenyl]-ethylamine is obtained as described in Example 4 using 3-chloro-4-(8-thiophen-2-yl-imidazo[4,5-c]quinolin-1-yl)-phenyl]-acetonitrile (Example 7) as starting material. 2-[3-Chloro-4-(8-thiophen-2-yl-imidazo[4,5-c]quinolin-1-yl)-phenyl]-ethylamine, analytical HPLC: t_{R}=5.46 minutes (Grad 1); ES⁺-MS: m/eₒ=405.1.

### Example 11

### 2-{3-Chloro-4-[8-(1H-indol-5-yl)-imidazo[4,5-c]quinolin-1-yl]-phenyl}-ethylamine

2-{3-Chloro-4-[8-(1H-indol-5-yl)-imidazo[4,5-c]quinolin-1-yl]-phenyl}-ethylamine is obtained as described in Example 4 using {4-[8-(1H-indol-5-yl)-imidazo[4,5-c]quinolin-1-yl]-phenyl}-acetonitrile (Example 8) as starting material. 2-{3-Chloro-4-[8-(1H-indol-5-yl)-imidazo[4,5-c]quinolin-1-yl]-phenyl}-ethylamine, analytical HPLC: t_{R}=5.61 minutes (Grad 1); ES⁺-MS: m/eₒ=438.3.

### Example 12

### 2-[3-Chloro-4-(8-thiophen-3-yl-imidazo[4,5-c]quinolin-1-yl)-phenyl]-ethylamine

2-[3-Chloro-4-(8-thiophen-3-yl-imidazo[4,5-c]quinolin-1-yl)-phenyl]-ethylamine is obtained as described in Example 4 using [3-chloro(8-thiophen-3-yl-imidazo[4,5-c]quinolin-1-yl)phenyl]-acetonitrile (Example 9) as starting material. 2-[3-Chloro-4-(8-thiophen-3-yl-imidazo[4,5-b]quinolin-1-yl)-phenyl]-ethylamine, analytical HPLC: t_{R}=5.50 minutes (Grad 1); ES⁺-MS: m/eₒ=405.1.

### Example 13

### [2-Fluoro-4-(8-thiophen-2-yl-imidazo[4,5-c]quinolin-1-yl)-phenyl]-acetonitrile

[2-Fluoro-4-(8-thiophen-2-yl-imidazo[4,5-c]quinolin-1-yl)-phenyl]-acetonitrile is obtained as described in Example 1 using (4-amino-2-fluoro-phenyl)-acetonitrile (Example 13a) and thiophene-2-boronic acid. [2-Fluoro-4-(8-thiophen-2-yl-imidazo[4,5-c]quinolin-1-yl)-phenyl]-acetonitrile, analytical HPLC: t_{R}=6.64 minutes (Grad 1); ES⁺-MS: m/eₒ=385.1.

### Example 13a

### (4-Amino-2-fluoro-phenyl)-acetonitrile

1.55 g (8.6 mmol) of (2-fluoro-4-nitro-phenyl)-acetonitrile (Example 13b) and 160 mg of Pd 5% on charcoal are shacked in 45 mL of MeOH under 1.1 bar of H₂ for 4 hours. After completion of the reaction, the catalyst is filtered-off and the filtrate is evaporated in vacuo to dryness to provide (4-amino-2-fluoro-phenyl)-acetonitrile as a brown solid, analytical HPLC: t_{R}=1.76 minutes (Grad 3).

¹H NMR (CDCl₃): δ 7.15 (t, 1H), 6.40-6.48 (m, 2H), 3.88 (br, s, 2H), 3.64 (s, 2H).

### Example 13b

### (2-Fluoro-4-nitro-phenyl)-acetonitrile

1.59 g (10 mmol) of 3,4-difluoro-1-nitrobenzene, 1.9 g (13.8 mmol) of finely-powdered K₂CO₃, 16.6 mg (0.1 mmol) of KI and 1.24 g (11 mmol) of ethyl cyanoacetate in 10 mL DMF are stirred for 4 hours at RT, and then 1 hour at 50° C. and 1 hour at 100° C. The reaction mixture is quenched with aqueous 1 M citric acid and extracted with EtOAc. The combined organic layers are washed with brine, dried over MgSO₄, filtered and evaporated in vacuo. The residue is treated with 1 mL HCl (37%) in 10 mL H₂O:acetic acid (3:1) for 8 hours at 100° C. After this time, the reaction mixture is quenched with saturated aqueous NaHCO₃ and extracted with ether. The combined organic layers are washed with aqueous NaHCO₃, brine and dried over MgSO₄. The organic phase is evaporated in vacuo to dryness to give (2-fluoro-4-nitro-phenyl)-acetonitrile as a pale yellow solid, analytical HPLC: t_{R}=3.69 minutes (Grad 2); ES⁻-MS: m/eₒ=178.9.

### Example 14

### [4-(8-Benzofuran-2-yl-imidazo[4,5-c]quinolin-1-yl)-2-fluoro-phenyl]-acetonitrile

[4-(8-Benzofuran-2-yl-imidazo[4,5-c]quinolin-1-yl)-2-fluoro-phenyl]-acetonitrile is obtained as described in Example 1 using (4-amino-2-fluoro-phenyl)-acetonitrile (Example 13a) and benzo[b]furan-2-boronic acid. [4-(8-Benzofuran-2-yl-imidazo[4,5-c]quinolin-1-yl)-2-fluoro-phenyl]-acetonitrile, analytical HPLC: t_{R}=7.43 minutes (Grad 1); ES⁺-MS: m/eₒ=419.1.

### Example 15

### {2-Fluoro-4-[8-(1H-indol-5-yl)-imidazo[4,5-c]quinolin-1-yl]-phenyl}-acetonitrile

{2-Fluoro-4-[8-(1H-indol-5-yl)-imidazo[4,5-c]quinolin-1-yl]-phenyl}-acetonitrile is obtained as described in Example 1 using (4-amino-2-fluoro-phenyl)-acetonitrile (Example 13a) and 5-indolylboronic acid. {2-Fluoro-4-[8-(1H-indol-5-yl)-imidazo[4,5-c]quinolin-1-yl]-phenyl}-acetonitrile, analytical HPLC: t_{R}=6.57 minutes (Grad 1); ES⁺-MS: m/eₒ=418.1.

### Example 16

### 2-[2-Fluoro-4-(8-thiophen-2-yl-imidazo[4,5-c]quinolin-1-yl)-phenyl]-ethylamine

2-[2-Fluoro-4-(8-thiophen-2-yl-imidazo[4,5c]quinolin-1-yl)-phenyl]-ethylamine is obtained as described in Example 4 using [2-fluoro-4-(8-thiophen-2-yl-imidazo[4,5-c]quinolin-1-yl)-phenyl]-acetonitrile (Example 13) as starting material. 2-[2-Fluoro-4-(8-thiophen-2-yl-imidazo[4,5-c]quinolin-1-yl)-phenyl]-ethylamine, analytical HPLC: t_{R}=5.44 minutes (Grad 1); ES⁺-MS: m/eₒ=389.1.

### Example 17

### 2-[4-(8-Benzofuran-2-yl-imidazo[4,5-c]quinolin-1-yl)-2-fluoro-phenyl]-ethylamine

2-[4-(8-Benzofuran-2-yl-imidazo[4,5-c]quinolin-1-yl)-2-fluoro-phenyl]-ethylamine is obtained as described in Example 4 using [4-(8-benzofuran-2-yl-imidazo[4,5-c]quinolin-1-yl)-2-fluoro-phenyl]-acetonitrile (Example 14) as starting material. 2-[4-(8-Benzofuran-2-yl-imidazo[4,5-c]quinolin-1-yl)-2-fluoro-phenyl]-ethylamine, analytical HPLC: t_{R}=5.93 minutes (Grad 1); ES⁺-MS: m/eₒ=423.1.

### Example 18

### 2-{2-Fluoro-4-[8-(1H-indol-5-yl)-imidazo[4,5-c]quinolin-1-yl]-phenyl}-ethylamine

2-{2-Fluoro-4-[8-(1H-indol-5-yl)-imidazo[4,5-c]quinolin-1-yl]-phenyl}-ethylamine is obtained as described in Example 4 using {2-fluoro-4-[8-(1H-indol-5-yl)-imidazo[4,5-c]quinolin-1-yl]-phenyl}-acetonitrile (Example 15) as starting material. 2-{2-Fluoro-4-[8-(1H-indol-5-yl)-imidazo[4,5-c]quinolin-1-yl]-phenyl}-ethylamine, analytical HPLC: t_{R}=5.54 minutes (Grad 1); ES⁺-MS: m/eₒ=422.1.

### Example 19

### [3-Methyl-4-(8-thiophen-3-yl-imidazo[4,5-c]quinolin-1-yl)-phenyl]-acetonitrile

[3-Methyl-4-(8-thiophen-3-yl-imidazo[4,5-c]quinolin-1-yl)-phenyl]-acetonitrile is obtained as described in Example 1 using (4-amino-3-methyl-phenyl)-acetonitrile (Example 19a) and thiophene-3-boronic acid. [3-Methyl-4-(8-thiophen-3-yl-imidazo[4,5-c]quinolin-1-yl)-phenyl]-acetonitrile, analytical HPLC: t_{R}=1.81 minutes (Grad 4); ES⁺-MS: m/eₒ=381.2.

### Example 19a

### (4-Amino-3-methyl-phenyl)-acetonitrile

(4-Amino-3-methyl-phenyl)-acetonitrile is obtained as described in Example 13a from (2-methyl-4-nitro-phenyl)-acetonitrile (Example 19b). (4-Amino-3-methyl-phenyl)-acetonitrile, analytical HPLC: t_{R}=1.73 minutes (Grad 3); ES⁺-MS: m/eₒ=146.9.

### Example 19b

### (2-Methyl-4-nitro-phenyl)-acetonitrile

0.83 g (13 mmol) of KOH and 1.47 g (13 mmol) of ethyl cyanoacetate in 4 mL DMSO are stirred for 1 hour and then 1.55 g (10 mmol) of 2-fluoro-5-nitrotoluene are added. The reaction mixture is stirred for 8 hours. After this time, 2.5 mL of water, 2.8 mL of acetic acid and 2.5 mL of HCl 37% are added and the reaction mixture is stirred for 2 hours at 100° C. After this time, water is added and the suspension is extracted with ether. The combined organic layers are washed with brine, dried over MgSO₄, filtered and evaporated in vacuo. The residue is purified by flash chromatography on silica gel (hexane:ethyl acetate 10:1 to 2:1) to give (2-methyl-4-nitro-phenyl)-acetonitrile as a brown solid, analytical HPLC: t_{R}=3.92 minutes (Grad 2); ES⁺-MS: m/eₒ 174.9.

### Example 20

### {4-[8-(1H-Indol-5-yl)-imidazo[4,5-c]quinolin-1-yl]-3-methyl-phenyl}-acetonitrile

{4-[8-(1H-indol-5-yl)-imidazo[4,5-c]quinolin-1-yl]-3-methyl-phenyl}-acetonitrile is obtained as described in Example 1 using (4-amino-3-methyl-phenyl)-acetonitrile (Example 19a) and 5-indolylboronic acid. {4-[8-(1H-Indol-5-yl)-imidazo[4,5-c]quinolin-1-yl]-3-methyl-phenyl}-acetonitrile, analytical HPLC: t_{R}=1.81 minutes (Grad 4); ES⁺-MS: m/eₒ=414.3.

### Example 21

### 2-[3-Methyl-4-(8-thiophen-3-yl-imidazo[4,5-c]quinolin-1-yl)-phenyl]-ethylamine

2-[3-Methyl-4-(8-thiophen-3-yl-imidazo[4,5-c]quinolin-1-yl)-phenyl]-ethylamine is obtained as described in Example 4 using [3-methyl-4-(8-thiophen-3-yl-imidazo[4,5-c]quinolin-1-yl)-phenyl]-acetonitrile (Example 19) as starting material. 2-[3-Methyl-4-(8-thiophen-3-yl-Imidazo[4,5-c]quinolin-1-yl)-phenyl]-ethylamine, analytical HPLC: t_{R}=1.30 minutes (Grad 4); ES⁺-MS: m/eₒ=385.1 (M+H)⁺.

### Example 22

### 2-{4-[8-(1H-indol-5-yl)-Imidazo[4,5-c]quinolin-1-yl]-3-methyl-phenyl}-ethylamine

2-{4-[8-(1H-indol-5-yl)-imidazo[4,5-c]quinolin-1-yl]-3-methyl-phenyl}-ethylamine is obtained as described in Example 4 using {4-[8-(1H-indol-5-yl)-imidazo[4,5-c]quinolin-1-yl]-3-methyl-phenyl}-acetonitrile (Example 20) as starting material. 2-{4-[8-(1H-indol-5-yl)-imidazo[4,5-c]quinolin-1-yl]-3-methyl-phenyl}-ethylamine, analytical HPLC: t_{R}=1.41 minutes (Grad 4); ES⁺-MS: m/eₒ=418.3.

### Example 23

### (R)-2-Amino-3-[4-(8-thiophen-2-yl-imidazo[4,5-c]quinolin-1-yl)-phenyl]-propionamide

(R)-2-Amino-3-[4-(8-thiophen-2-yl-imidazo[4,5c]quinolin-1-yl)-phenyl]-propionamide is obtained as described in Example 1 using (R)-[2-(4-amino-phenyl)-1-carbamoyl-ethyl]-carbamic acid tert-butyl ester (Example 23a) and thiophene-2-boronic acid, followed by a subsequent treatment with TFA to remove the Boc-protecting group. (R)-2-Amino-3-[4-(8-thiophen-2-yl-imidazo[4,5-c]quinolin-1-yl)-phenyl]-propionamide, analytical HPLC: t_{R}=2.30 minutes (Grad 3); ES⁺-MS: m/eₒ32 414.0.

### Example 23a

### (R)-[2-(4-Amino-phenyl)-1-carbamoyl-ethyl]-carbamic acid tert-butyl ester

(R)-[2-(4-Amino-phenyl)-1-carbamoyl-ethyl]-carbamic acid tert-butyl ester is obtained by reduction of (R)-[1-carbamoyl-2-(4-nitro-phenyl)-ethyl]-carbamic acid tert-butyl ester (Example 23b) as described in Example 13a. (R)-[2-(4-Amino-phenyl)-1-carbamoyl-ethyl]-carbamic acid tert-butyl ester, analytical HPLC: t=2.08 minutes (Grad 3); ES⁺-MS: m/eₒ=280.1.

### Example 23b

### (R)-[1-Carbamoyl-2-(4-nitro-phenyl)-ethyl]-carbamic acid tert-butyl ester

To a solution of 1.0 g (3.2 mmol) of (R-2-tert-butoxycarbonylamino-3-(4-nitro-phenyl)-propionic acid in 8 mL of dimethylacetamide is added 905 mg (7 mmol) of diisopropylethylamine and 998 mg (3.36 mmol) of O-(1,2-dihydro-2-oxo-1-pyridyl)-N,N,N′,N′-tetramethyluronium tetrafluoroborate. Ater 5 minutes, NH₃ gas is bubbled for 3 minutes in the reaction mixture. The reaction mixture is stirred for 3 minutes, quenched with aqueous 1 M citric acid and extracted with EtOAc. The combined organic layers are washed with water and with brine, dried over MgSO₄, filtered and evaporated in vacuo. The residue is purified by flash chromatography on silica gel [hexane-EtOAc (2:1)] to give (R)-[1-carbamoyl-2-(4-nitro-phenyl)-ethyl]-carbamic acid tert-butyl ester as a white solid, analytical HPLC: t_{R}=3.59 minutes (Grad 2); ES⁺-MS: m/eₒ=310.0.

### Example 24

### (R)-2-Amino-3-[4-(8-benzo[b]thiophen-2-yl-imidazo[4,5-c]quinolin-1-yl)-phenyl]-propionamide

(R)-2-Amino-3-[4-(8-benzo[b]thiophen-2-yl-imidazo[4,5c]quinolin-1-yl)-phenyl]-propionamide is obtained as described in Example 1 using (R)-[2-(4-amino-phenyl)-1-carbamoyl-ethyl]-carbamic acid tert-butyl ester (Example 23a) and benzo[b]thiophene-2-boronic acid, followed by a subsequent treatment with TFA to remove the Boc-protecting group. 2-Amino-3-[4-(8-benzo[b]thiophen-2-yl-imidazo[4,5-c]quinolin-1-yl)-phenyl]-propionamide, analytical HPLC: t_{R}_{=2.83} minutes (Grad 2); ES⁺-MS: m/eₒ=464.0.

### Example 25

### [3,5-Dichloro-4-(8-thiophen-2-yl-imidazo[4,5-c]quinolin-1-yl)-phenyl]-acetonitrile

[3,5-Dichloro-4-(8-thiophen-2-yl-imidazo[4,5-c]quinolin-1-yl)-phenyl]-acetonitrile is prepared as described in Example 1 using (4-amino-3,5-dichloro-phenyl)-acetonitrile (Example 25a) and thiophene-2-boronic acid. [3,5-Dichloro-4-(8-thiophen-2-yl-imidazo[4,5-c]quinolin-1-yl)phenyl]-acetonitrile, analytical HPLC: t_{R}=3.94 minutes (Grad 2); ES⁺-MS: m/eₒ=434.8, 436.7, 438.8.

### Example 25a

### (4-Amino-3,5-dichloro-phenyl)-acetonitrile

2 g (15.1 mmol) of (4-amino-phenyl)-acetonitrile and N-chlorosuccinimide in 30 mL of methanol are stirred for 2 hours at 0° C. and 15 hours at RT. After this time, the reaction mixture is concentrated in vacuo, quenched with saturated aqueous NaHCO₃ and extracted with EtOAc. The organic layer is washed with brine, dried over MgSO₄, filtered and evaporated in vacuo. The residue is purified by flash chromatography on silica gel [hexane:EtOAc (4:1) to (3:1)] to give (4-amino-3,5-dichloro-phenyl)-acetonitrile as an off-white solid, 2 g (15.1 mmol) of (4-amino-phenyl)-acetonitrile, analytical HPLC: t_{R}=4.08 minutes (Grad 2).

¹H NMR (DMSO-d₆): δ 7.23 (s, 2H), 5.59 (br, s, 2H), 3.85 (s, 2H).

### Example 26

### [3,5-Dichloro-4-(8-thiophen-3-yl-Imidazo[4,5-c]quinolin-1-yl)-phenyl]-acetonitrile

[3,5-Dichloro-4-(8-thiophen-3-yl-imidazo[4,5-c]quinolin-1-yl)-phenyl]-acetonitrile is obtained as described in Example 1 using (4-amino-3,5-dichloro-phenyl)-acetonitrile (Example 25a) and thiophene-3-boronic acid. [3,5-Dichloro-4-(8-thiophen-3-yl-imidazo[4,5-c]quinolin-1-yl)-phenyl]-acetonitrile, analytical HPLC: t_{R}=3.86 minutes (Grad 2); ES⁺-MS: m/eₒ=434.8, 436.8, 438.8.

### Example 27

### 2-[3,5-Dichloro-4-(8-thiophen-2-yl-imidazo[4,5-c]quinolin-1-yl)-phenyl]-ethylamine

2-[3,5-Dichloro-(8-thiophen-2-yl-imidazo[4,5-c]quinolin-1-yl)-phenyl]-ethylamine is obtained as described in Example 4 using [3,5-dichloro-4-(8-thiophen-2-yl-imidazo[4,5-c]quinolin-1-yl)-phenyl]-acetonitrile (Example 25) as starting material. 2-[3,5-Dichloro-4-(8-thiophen-2-yl-Imidazo[4,5-c]quinolin-1-yl)-phenyl]-ethylamine, analytical HPLC: t_{R}=2.80 minutes (Grad 2); ES⁺-MS: m/eₒ=438.8, 440.8, 442.8.

### Example 28

### 2-[3,5-Dichloro-4-(8-thiophen-3-yl-imidazo[4,5-c]quinolin-1-yl)-phenyl]-ethylamine

2-[3,5-Dichloro-4-(8-thiophen-3-yl-imidazo[4,5-c]quinolin-1-yl)phenyl]-ethylamine is obtained as described in Example 4 using [3,5-dichloro-4-(8-thiophen-3-yl-imidazo[4,5-c]quinolin-1-yl)-phenyl]-acetonitrile (Example 26) as starting material. 2-[3,5-Dichloro-4-(8-thiophen-3-yl-imidazo[4,5-c]quinolin-1-yl)-phenyl]-ethylamine, analytical HPLC: t_{R}=2.74 minutes (Grad 2); ES⁺-MS: m/eₒ=438.8, 440.8, 442.8.

### Example 29

### {4-[8-(4-Hydroxy-phenyl)-imidazo[4,5-c]quinolin-1-yl]-3-trifluoromethyl-phenyl}-acetonitrile

{4-[8-(4-Hydroxy-phenyl)-imidazo[4,5-c]quinolin-1-yl]-3-trifluoromethyl-phenyl}-acetonitrile is obtained as described in Example 1 using (4-amino-3-trifluoromethyl-phenyl)-acetonitrile (prepared after a procedure in Eur J Med Chem, Vol. 31, p. 133 (1996) and 4-hydroxyphenylboronic acid. {4-[8-(4-Hydroxy-phenyl)imidazo[4,5-c]quinolin-1-yl]-3-trifluoromethyl-phenyl}-acetonitrile, analytical HPLC: t_{R}=2.48 minutes (Grad 5); ES⁺-MS: m/eₒ=445.0.

### Example 30

### 4-{1-[4-(2-Amino-ethyl)-2-trifluoromethyl-phenyl]-1H-imidazo[4,5-c]quinolin-8-yl}-phenol

4-{1-[4-(2-Amino-ethyl-2-trifluoromethyl-phenyl]-1H-imidazo[4,5-c]quinolin-8-yl}-phenol is obtained as described in Example 4 using {4-[8-(4-hydroxy-phenyl)-imidazo[4,5-c]quinolin-1-yl]-3-trifluoromethyl-phenyl}-acetonitrile (Example 29) as starting material. 4-{1-[4-(2-Amino-ethyl)-2-trifluoromethyl-phenyl]-1H-imidazo[4,5-c]quinolin-8-yl}-phenol, analytical HPLC: t_{R}=6.69 minutes (Grad 6); ES⁺-MS: m/eₒ=449.1.

### Example 31

### 3-[4-(8-Benzo[1,3]dioxol-5-yl-imidazo[4,5-c]quinolin-1-yl)-phenyl]-propionitrile

3-[4-(8-Benzo[1,3]dioxol-5-yl-imidazo[4,5-c]quinolin-1-yl)-phenyl]-propionitrile is obtained as described in Example 1 using 3-(4-amino-phenyl)propionitrile (Example 31a). 3-[4-(8-Benzo[1,3]dioxol-5-yl-imidazo[4,5-c]quinolin-1-yl)-phenyl]-propionitrile, analytical HPLC: t_{R}=7.70 minutes (Grad 1); ES⁺-MS: m/eₒ=419.2 (M+H)⁺.

### Example 31a

### 3-(4-Amino-phenyl)-propionitrile

0.78 g (4.4 mmol) of 3-(4-nitro-phenyl)-propionitrile (Example 31b) are dissolved in 40 mL of MeOH:THF (1:1) and hydrogenated at RT in the presence of 50 mg of Pd—C 10%. After completion of the reaction, the catalyst is filtered-off and washed with methanol. The organic solvent is evaporated to dryness to provide 3-(4-amino-phenyl)-propionitrile, analytical HPLC: t_{R}=4.81 minutes (Grad 1); ES⁺-MS: m/eₒ=147.3.

### Example 31b

### 3-(4-Nitro-phenyl)-propionitrile

3.45 of (15 mmol) of 4-nitrophenethyl bromide are dissolved in 50 mL of ethanol and 0.81 g (16.5 mmol) of sodium cyanide are added. The solution is stirred for 4 hours at RT and then evaporated to dryness. The crude compound is dissolved in 100 mL of ethyl acetate, and the organic solution is extracted with water, brine, dried over MgSO₄ and evaporated to dryness. The crude compound is purified by medium-pressure liquid chromatography to provide 3-(4-nitro-phenyl)-propionitrile, analytical HPLC: t_{R}=7.27 minutes (Grad 1); ES−MS: m/eₒ=175.3.

### Example 32

### 3-[4-(8-Thiophen-2-yl-imidazo[4,5-c]quinolin-1-yl)-phenyl]-propionitrile

3-[4-(8-Thiophen-2-yl-imidazo[4,5-c]quinolin-1-yl)-phenyl]-propionitrile is obtained as described in Example 1 using 3-(4-amino-phenyl)-propionitrile and thiophene-2-boronic acid. 3-[4-(8-Thiophen-2-yl-imidazo[4,5-c]quinolin-1-yl)-phenyl]-propionitrile, analytical HPLC: t_{R}=7.70 minutes (Grad 1); ES⁺-MS: m/eₒ=381.1.

### Example 33

### 3-[4-(8-Benzo[b]thiophen-2-yl-imidazo[4,5-c]quinolin-1-yl)-phenyl]-propionitrile

3-[4-(8-Benzo[b]thiophen-2-yl-imidazo[4,5-c]quinolin-1-yl)phenyl]-propionitrile is obtained as described in Example 1 using 3-(4-amino-phenyl)-propionitrile (Example 31a) and benzo[b]thiophene-2-boronic acid. 3-[4-(8-Benzo[b]thiophen-2-yl-imidazo[4,5-c]quinolin-1-yl)-phenyl]-propionitrile, analytical HPLC: t_{R}=8.23 minutes (Grad 1); ES⁺-MS: m/eₒ=431.1.

### Example 34

### 3-[4-(8-Thiophen-3-yl-imidazo[4,5-c]quinolin-1-yl)-phenyl]-propionitrile

3-[4-(8-Thiophen-3-yl-imidazo[4,5-c]quinolin-1-yl)-phenyl]-propionitrile is obtained as described in Example 1 using 3-(4-amino-phenyl)-propionitrile and thiophene-3-boronic acid. 3-[4-(8-Thiophen-3-yl-imidazo[4,5c]quinolin-1-yl)-phenyl]-propionitrile, analytical HPLC: t_{R}=7.63 minutes (Grad 1); ES⁺-MS: m/eₒ=381.3 (M+H)⁺.

### Example 35

### 3-[4-(8-Benzofuran-2-yl-imidazo[4,5-c]quinolin-1-yl)-phenyl]-propionitrile

3-[4-(8-Benzofuran-2-yl-imidazo[4,5-c]quinolin-1-yl)-phenyl]-propionitrile is obtained as described in Example 1 using 3-(4-amino-phenyl)propionitrile (Example 31a) and benzo[b]furan-2-boronic acid. 3-[4-(8-Benzofuran-2-yl-imidazo[4,5-c]quinolin-1-yl)-phenyl]-propionitrile, analytical HPLC: t_{R}=8.43 minutes (Grad 1); ES⁺-MS: m/eₒ=415.5.

### Example 36

### 3-[4-(8-Benzo[1,3]-dioxol-5-yl-imidazo[4,5-c]quinolin-1-yl)-phenyl]-propylamine

3-[4-(8-Benzo[1,3]dioxol-5-yl-imidazo[4,5-c]quinolin-1-yl)-phenyl]-propylamine is obtained as described in Example 4 using 3-[4-(8-benzo[1,3]dioxol-5-yl-imidazo[4,5-c]quinolin-1-yl)-phenyl]-propionitrile (Example 31) as starting material. 3-[4-(8-Benzo[1,3]dioxol-5-yl-imidazo[4,5-c]quinolin-1-yl)phenyl]-propylamine, analytical HPLC: t_{R}=7.02 minutes (Grad 1); ES⁺-MS: m/eₒ=423.1.

### Example 37

### 3-[4-(8-Thiophen-2-yl-imidazo[4,5-c]quinolin-1-yl)-phenyl]-propylamine

3-[4-(8-Thiophen-2-yl-imidazo[4,5-c]quinolin-1-yl)phenyl]-propylamine is obtained as described in Example 4 using 3-[4-(8-thiophen-2-yl-imidazo[4,5-c]quinolin-1-yl)-phenyl]-propionitrile (Example 32) as starting material. 3-[4-(8-Thiophen-2-yl-imidazo[4,5-c]quinolin-1-yl)-phenyl]-propylamine, analytical HPLC: t_{R}=6.99 minutes (Grad 1); ES⁺-MS: m/eₒ=385.2.

### Example 38

### 3-[4-(8-Benzo[b]thiophen-2-yl-imidazo[4,5-c]quinolin-1-yl)-phenyl]-propylamine

3-[4-(8-Benzo[b]thiophen-2-yl-imidazo[4,5-c]quinolin-1-yl)-phenyl]-propylamine is obtained as described in Example 4 using 3-[4-(8-benzo[b]thiophen-2-yl-imidazo[4,5-c]quinolin-1-yl)-phenyl]-propionitrile (Example 33) as starting material. 3-[4-(8-Benzo[b]thiophen-2-yl-imidazo[4,5-c]quinolin-1-yl)phenyl]-propylamine: analytical HPLC: t_{R}=7.20 minutes (Grad 1); ES⁺-MS: m/eₒ=435.1.

### Example 39

### 3-[4-(8-Thiophen-3-yl-imidazo[4,5-c]quinolin-1-yl)-phenyl]-propylamine

3-[4-(8-Thiophen-3-yl-imidazo[4,5-c]quinolin-1-yl)-phenyl]-propylamine is obtained as described in Example 4 using 3-[4-(8-thiophen-3-yl-imidazo[4,5-c]quinolin-1-yl)-phenyl]-propionitrile (Example 34) as starting material. 3-[4-(8-Thiophen-3-yl-imidazo[4,5-c]quinolin-1-yl)-phenyl]-propylamine: analytical HPLC: t_{R}=5.71 minutes (Grad 1); ES⁺-MS: m/eₒ=385.2.

### Example 40

### 3-[4-(8-Benzofuran-2-yl-imidazo[4,5-c]quinolin-1-yl)-phenyl]-propylamine

3-[4-(8-Benzofuran-2-yl-imidazo[4,5-c]quinine-1-yl)-phenyl]-propylamine is obtained as described in Example 4 using 3-[4-(8-benzofuran-2-yl-imidazo[4,5-c]quinolin-1-yl)-phenyl]-propionitrile (Example 35) as starting material. 3-[4-(8-Benzofuran-2-yl-imidazo[4,5-c]quinolin-1-yl)-phenyl]-propylamine, analytical HPLC: t_{R}=6.07 minutes (Grad 1); ES⁺-MS: m/eₒ=419.2.

### Example 41

### 3-[4-(8-Benzo[1,3]dioxol-5-yl imidazo[4,5-c]quinolin-1-yl)-3-chloro-phenyl]-propionitrile

3-[4-(8-Benzo[1,3]dioxol-5-yl-imidazo[4,5-c]quinolin-1-yl)-3-chloro-phenyl]-propionitrile is obtained as described in Example 1 using 3-(4-amino-3-chloro-phenyl)-propionitrile. 3-[4-(8-Benzo[1,3]dioxol-5-yl-imidazo[4,5-c]quinolin-1-yl)-3-chloro-phenyl]-propionitrile, analytical HPLC: t_{R}=6.90 minutes (Grad 1); ES⁺-MS: m/eₒ=453.3.

### Example 41a

### 3-(4-Amino-3-chloro-phenyl)-propionitrile

0.62 mg of 3-(4-amino-phenyl)-propionitrile (Example 31a) are dissolved in 7 mL of isopropanol and 0.6 g (4.48 mmol) of N-chlorosuccinimide are added. The solution is refluxed for 30 minutes and then the solvent is evaporated to dryness. The crude compound is purified by medium-pressure liquid chromatography to provide 3-(4-amino-3-chloro-phenyl)-propionitrile, analytical HPLC: t_{R}=6.42 minutes (Grad 1); ES⁺-MS: m/eₒ=180.9.

### Example 42

### 3-[3-Chloro-4-(8-thiophen-2-yl-imidazo[4,5-c]quinolin-1-yl)-phenyl]-propionitrile

3-[3-Chloro-4-(8-thiophen-2-yl-imidazo[4,5-c]quinolin-1-yl)-phenyl]-propionitrile is obtained as described in Example 1 using 3-(4-amino-3-chloro-phenyl)-propionitrile and thiophene-2-boronic acid. 3-[3-Chloro-(8-thiophen-2-yl-imidazo[4,5-c]quinolin-1-yl)-phenyl]-propionitrile, analytical HPLC: t_{R}=6.85 minutes (Grad 1); ES⁺-MS: m/eₒ=415.0.

### Example 43

### 3-[3-Chloro-4-(8-thiophen-3-yl-imidazo[4,5-c]quinolin-1-yl)-phenyl]-propionitrile

3-[3-Chloro-4-(8-thiophen-3-yl-imidazo[4,5-c]quinolin-1-yl)-phenyl]-propionitrile is obtained as described in Example 1 using 3-(4-amino-3-chloro-phenyl)-propionitrile and thiophene-3-boronic acid. 3-[3-Chloro-4-(8-thiophen-3-yl-imidazo[4,5-c]quinolin-1-yl)-phenyl]-propionitrile, analytical HPLC: t_{R}=6.76 minutes (Grad 1); ES⁺-MS: m/eₒ=415.0.

### Example 44

### 3-{3-Chloro-4-[8-(1H-indol-5-yl)-imidazo[4,5-c]quinolin-1-yl]-phenyl}-propionitrile

3-{3-Chloro-4-[8-(1H-indol-5-yl)-imidazo[4,5-c]quinolin-1-yl]-phenyl}-propionitrile is obtained as described in Example 1 using 3-(4-amino-3-chloro-phenyl) propionitrile and 5-indolylboronic acid. 3-{3-Chloro-4-[8-(1H-indol-5-yl)-imidazo[4,5-c]quinolin-1-yl]-phenyl}-propionitrile, analytical HPLC: t_{R}=6.73 minutes (Grad 1); ES⁺-MS: m/eₒ=448.1.

### Example 45

### 3-[4-(8-Benzo[1,3]dioxol-5-yl-imidazo[4,5-c]quinolin-1-yl)-3-chloro-phenyl]-propylamine

3-[4-(8-Benzo[1,3]dioxol-5-yl-imidazo[4,5-c]quinolin-1-yl)-3-chloro-phenyl]-propylamine is synthesized as described in Example 4 using 3-[4-(8-benzo[1,3]dioxol-5-yl-imidazo[4,5-c]quinolin-1-yl)-3-chloro-phenyl]-propionitrile (Example 41) as starting material. 3-[4-(8-Benzo[1,3]dioxol-5-yl-imidazo[4,5-c]quinolin-1-yl)-3-chloro-phenyl]-propylamine, analytical HPLC: t_{R}=5.71 minutes (Grad 1); ES⁺-MS: m/eₒ=457.3.

### Example 46

### 3-[3-Chloro-4-(8-thiophen-2-yl-Imidazo[4,5-c]quinolin-1-yl)-phenyl]-propylamine

3-[3-Chloro-4-(8-thiophen-2-yl-imidazo[4,5-c]quinolin-1-yl)-phenyl]-propylamine is synthesized as described in Example 4 using 3-[3-chloro-4-(8-thiophen-2-yl-imidazo[4,5-c]quinolin-1-yl)-phenyl]-propionitrile (Example 42) as starting material. 3-[3-Chloro-4-(8-thiophen-2-yl-imidazo[4,5-c]quinolin-1-yl)-phenyl]-propylamine, analytical HPLC: t_{R}=5.63 minutes (Grad 1); ES⁺-MS: m/eₒ=419.3.

### Example 47

### 3-[3-Chloro-4-(8-thiophen-3-yl-imidazo[4,5-c]quinolin-1-yl)-phenyl]-propylamine

3-[3-Chloro-4-(8-thiophen-3-yl-imidazo[4,5-c]quinolin-1-yl)-phenyl]-propylamine is synthesized as described in Example 4 using 3-[3-chloro-4-(8-thiophen-3-yl-imidazo[4,5-c]quinolin-1-yl)-phenyl]-propionitrile (Example 43) as starting material. 3-[3-Chloro-4-(8-thiophen-3-yl-imidazo[4,5-c]quinolin-1-yl)-phenyl]-propylamine, analytical HPLC: t_{R}=5.58 minutes (Grad 1); ES⁺-MS: m/eₒ=419.1.

### Example 48

### 3-{3-Chloro-4-[8-(1H-indol-5-yl)-imidazo[4,5-c]quinolin-1-yl]-phenyl}-propylamine

3-{3-Chloro-4-[8-(1H-indol-5-yl)-imidazo[4,5-c]quinolin-1-yl]-phenyl}-propylamine is obtained as described in Example 4 using 3-{3-chloro-4-[8-(1H-indol-5-yl)imidazo[4,5-c]quinolin-1-yl]-phenyl}propionitrile (Example 44) as starting material. 3-{3-Chloro-4-[8-(1H-indol-5-yl)-imidazo[4,5-c]quinolin-1-yl]-phenyl}-propylamine, analytical HPLC: t_{R}=5.69 minutes (Grad 1); ES⁺-MS: m/eₒ=452.1.

### Example 49

### 8-Benzo[1,3]dioxol-5-yl-1-{4-[2-(4,5-dihydro-1H-imidazol-2-yl)-ethyl]-phenyl}-1H-imidazo[4,5-c]quinoline

8-Benzo[1,3]dioxol-5-yl-1-{4-[2-(4,5-dihydro-1H-imidazol-2-yl)-ethyl]-phenyl}-1H-imidazo[4,5-c]quinoline is obtained as described in Example 1 using 4-[2-(4,5-dihydro-1H-imidazol-2-yl)-ethyl]-phenylamine (Example 49a). 8-Benzo[1,3]dioxol-5-yl-1-{4-[2-(4,5-dihydro-1H-imidazol-2-yl)-ethyl]-phenyl}-1H-imidazo[4,5-c]quinoline, analytical HPLC: t_{R}=6.35 minutes (Grad 1); ES⁺-MS: m/eₒ=462.2.

### Example 49a

### 4-[2-(4,5-Dihydro-1H-imidazol-2-yl)-ethyl]-phenylamine hydrogen chloride salt

0.66 g (2.6 mmol) of 2-[2-(4-nitrophenyl)-ethyl]-4,5-dihydro-1H-imidazole hydrogen chloride salt (Example 49c) are dissolved in 30 mL of methanol and hydrogenated at RT in the presence of 100 mg of Pd/C 10%. After completion of the reaction, the catalyst is filtered-off and washed with methanol. The organic solvent is concentrated to dryness to provide 4-[2-(4,5-dihydro-1H-imidazol-2-yl)-ethyl]-phenylamine hydrogen chloride salt, analytical HPLC: t_{R}=4.94 minutes (Grad 1); ES⁺-MS: m/eₒ=190.1.

### Example 49b

### 3-(4-Nitro-phenyl)-propionitrile

4.6 g (20 mmol) of 4-nitrophenethyl bromide and 0.98 g (20 mmol) of sodium cyanide are dissolved in 50 mL of ethanol and the solution is refluxed for 18 hours. After this time, the solution is concentrated to dryness and the crude compound is purified by chromatography on silica eluting with DCM to afford 3-(4-nitro-phenyl)-propionitrile, analytical HPLC: t_{R}=7.27 minutes (Grad 1); ES⁻-MS: m/eₒ=176.3.

### Example 49c

### 2-[2-(4-Nitro-phenyl)-ethyl]-4,5-dihydro-1H-imidazole hydrogen chloride salt

1.6 g (9.08 mmol) of 3-(4-nitro-phenyl)-propionitrile (Example 49b) are dissolved in 20.5 mL of a solution of DCM:ethanol (37:1) at 0° C. HCl-gas is bubbled in the reaction mixture for 20 minutes and then the solution is stirred for 18 hours at RT. After this time, 200 mL of diethyl ether are added and the precipitate is removed by filtration to provide 0.97 g (3.75 mmol) of 3-(4-nitro-phenyl)-propionimidic acid ethyl ester hydrogen chloride salt. This compound is dissolved in 30 mL of ethanol and 0.27 mL (4.01 mmol) of ethylendiamine are added. The solution is refluxed for 18 hours and then 20 mL of water are added. The pH of the solvent is adjusted to pH 1 with concentrated HCl. The solution is extracted with DCM and the organic phase is discarded. The pH of the aqueous phase is adjusted to pH 10 with 1 N NaOH, and extracted with DCM. The organic solution is extracted with brine, dried over MgSO₄ and evaporated to dryness to provide 2-[2-(4-nitro-phenyl)-ethyl]4,5-dihydro-1H-imidazole hydrogen chloride salt. The HCl salt is obtained by titration with 1.25 M HCl in methanol. 2-[2-(4-Nitro-phenyl)-ethyl]-4,5-dihydro-1H-imidazole hydrogen chloride salt, analytical HPLC: t_{R}=7.05 minutes (Grad 1); ES⁻-MS: m/eₒ=220.1.

### Example 50

### [3-Chloro-4-(2-methyl-8-thiophen-2-yl-imidazo[4,5-c]quinolin-1-yl)-phenyl]-acetonitrile

[3-Chloro-(2-methyl-8-thiophen-2-yl-imidazo[4,5-c]quinolin-1-yl)-phenyl]-acetonitrile is obtained as described in Example 1 using (4-amino-3-chloro-phenylyacetonitrile (Example 7a), triethyorthoacetate and thiophene-2-boronic acid. [3-Chloro-4-(2-methyl-8-thiophen-2-yl-imidazo[4,5-c]quinolin-1-yl)-phenyl]-acetonitrile, analytical HPLC: t_{R}=3.67 minutes (Grad 2); ES⁺-MS: m/eₒ=414.9.

### Example 51

### [3-Chloro-4-(2-methyl-8-thiophen-3-yl-imidazo[4,5-c]quinolin-1-yl)-phenyl]-acetonitrile

[3-Chloro-4-(2-methyl-8-thiophen-3-yl-imidazo[4,5-c]quinolin-1-yl)-phenyl]-acetonitrile is obtained as described in Example 1 using (4-amino-3-chloro-phenyl)-acetonitrile (Example 7a), triethyorthoacetate and thiophene-3-boronic acid. [3-Chloro-4-(2-methyl-8-thiophen-3-yl-imidazo[4,5-c]quinolin-1-yl)-phenyl]-acetonitrile, analytical HPLC: t_{R}=3.67 minutes (Grad 2); ES⁺-MS: m/eₒ=414.9.

### Example 52

### 2-[3-Chloro-4-(2-methyl-8-thiophen-2-yl-imidazo[4,5-c]quinolin-1-yl)-phenyl]-ethylamine

2-[3-Chloro-4-(2-methyl-8-thiophen-2-yl-imidazo[4,5-c]quinolin-1-yl)phenyl]-ethylamine is obtained as described in Example 4 using [3-chloro-4-(2-methyl-8-thiophen-2-yl-imidazo[4,5-c]quinolin-1-yl)-phenyl]-acetonitrile (Example 50) as starting material. 2-[3-Chloro-4-(2-methyl-8-thiophen-2-yl-imidazo[4,5-c]quinolin-1-yl)-phenyl]-ethylamine, analytical HPLC: t_{R}=2.61 minutes (Grad 2); ES⁺-MS: m/eₒ=418.9.

### Example 53

### 2-[3-Chloro-4-(2-methyl-8-thiophen-3-yl-imidazo[4,5-c]quinolin-1-yl)-phenyl]-ethylamine

2-[3-Chloro-4-(2-methyl-8-thiophen-3-yl-imidazo[4,5-c]quinolin-1-yl)phenyl]-ethylamine is obtained as described in Example 4 using [3-chloro-4-(2-methyl-8-thiophen-3-yl-imidazo[4,5-c]quinolin-1-yl)-phenyl]-acetonitrile (Example 51) as starting material. 2-[3-Chloro-4-(2-methyl-8-thiophen-3-yl-imidazo[4,5-c]quinolin-1-yl)-phenyl]-ethylamine, analytical HPLC: t_{R}=2.58 minutes (Grad 2); ES⁺-MS: m/eₒ=418.9.

### Example 54

### [4-(8-Benzo[1,3]dioxol-5-yl-7-fluoro-imidazo[4,5-c]quinolin-1-yl)-phenyl]-acetonitrile

[4-(8-Benzo[1,3]dioxol-5-yl-7-fluoro-imidazo[4,5-c]quinolin-1-yl)-phenyl]-acetonitrile is obtained as described in Example 1 using 6-bromo-4-chloro-7-fluoro-3-nitro-quinoline (Example 54a). [4-(8-Benzo[1,3]dioxol-5-yl-7-fluoro-imidazo[4,5c]quinolin-1-yl)-phenyl]-acetonitrile, analytical HPLC: t_{R}=7.01 minutes (Grad 1); ES⁺-MS: m/eₒ=423.2.

### Example 54a

### 6-Bromo-4-chloro-7-fluoro-3-nitro-quinoline

6-Bromo-4-chloro-7-fluoro-3-nitro-quinoline is obtained in analogy to 6-bromo-4-chloro-3-nitro-quinoline (Example 1c) starting from 2-amino-5-bromo-4-fluoro-benzoic acid (Example 54b). 6-Bromo-4-chloro-7-fluoro-3-nitro-quinoline, analytical HPLC: t_{R}=2.30 minutes (Grad 7); ES⁻-MS: m/eₒ=287.1.

### Example 54b

### 2-amino-5-bromo-4-fluoro-benzoic acid

20 g (128.9 mmol) of 2-amino-4-fluorobenzoic acid are dissolved in 470 mL of methanol and the solution is cooled at −70° C. To this stirred solution, 6.59 mL (128.2 mmol) of bromine dissolved in 130 mL of methanol are added slowly. After 3 hours, the solution is added to ice-water and the aqueous phase is extracted with ether. The combined organic portions are washed with water, brine, dried over MgSO₄ and concentrated in vacuo to provide 2-amino-5-bromo-4-fluoro-benzoic acid, analytical HPLC: t_{R}=2.96 minutes (Grad 5).

¹H NMR (DMSO-d₆): δ 7.85 (d, 1H), 6.64 (d, 1H).

### Example 55

### [4-(7-Fluoro-8-thiophen-2-yl-Imidazo[4,5-c]quinolin-1-yl)-phenyl]-acetonitrile

[4-(7-Fluoro-8-thiophen-2-yl-imidazo[4,5-c]quinolin-1-yl)-phenyl]-acetonitrile is obtained as described in Example 1 using 6-bromo-4-chloro-7-fluoro-3-nitro-quinoline (Example 54a) and thiophene-2-boronic acid. [4-(7-Fluoro-8-thiophen-2-yl-imidazo[4,5-c]quinolin-1-yl)-phenyl]-acetonitrile, analytical HPLC: t_{R}=7.20 minutes (Grad 1); ES⁺-MS: m/eₒ=385.1.

### Example 56

### [4-(7-Fluoro-8-thiophen-3-yl-imidazo[4,5-c]quinolin-1-yl)-phenyl]-acetonitrile

[4-(7-Fluoro-8-thiophen-3-yl-imidazo[4,5-c]quinolin-1-yl)-phenyl]-acetonitrile is obtained as described in Example 1 using 6-bromo-4-chloro-7-fluoro-3-nitro-quinoline (Example 54a) and thiophene-3-boronic acid. [4-(7-Fluoro-8-thiophen-3-yl-imidazo[4,5-c]quinolin-1-yl)-phenyl]-acetonitrile, analytical HPLC: t_{R}=7.06 minutes (Grad 1); ES⁺-MS: m/eₒ=385.0.

### Example 57

### [4-(8-Benzofuran-2-yl-7-fluoro-imidazo[4,5-c]quinolin-1-yl)-phenyl]-acetonitrile

[4-(8-Benzofuran-2-yl-7-fluoro-imidazo[4,5-c]quinolin-1-yl)-phenyl]-acetonitrile is obtained as described in Example 1 using 6-bromo-chloro-7-fluoro-3-nitro-quinoline (Example 54a) and benzo[b]furan-2-boronic acid. [4-(8-Benzofuran-2-yl-7-fluoro-imidazo[4,5-c]quinolin-1-yl)phenyl]-acetonitrile, analytical HPLC: t_{R}=8.41 minutes (Grad 1); ES⁺-MS: m/eₒ=419.1.

### Example 58

### {4-[7-Fluoro-8-(1H-indol-5-yl)-imidazo[4,5-c]quinolin-1-yl]-phenyl}-acetonitrile

{4-[7-Fluoro-8-(1H-indol-5-yl)-imidazo[4,5-c]quinolin-1-yl]-phenyl}-acetonitrile is obtained as described in Example 1 using 6-bromo-4-chloro-7-fluoro-3-nitro-quinoline (Example 54a) and 5-indolylboronic acid. {4-[7-Fluoro-8-(1H-indol-5-yl)-imidazo[4,5-c]quinolin-1-yl]-phenyl}-acetonitrile, analytical HPLC: t_{R}=6.79 minutes (Grad 1); ES⁺-MS: m/eₒ=418.2.

### Example 59

### [4-(8-Benzo[b]thiophen-2-yl-7-fluoro-imidazo[4,5-c]quinolin-1-yl)-phenyl]-acetonitrile

[4-(8-Benzo[b]thiophen-2-yl-7-fluoro-imidazo[4,5-c]quinolin-1-yl)-phenyl]-acetonitrile is obtained as described in Example 1 using 6-bromo-4-chloro-7-fluoro-3-nitro-quinoline (Example 54a) and benzo[b]thiophene-2-boronic acid. [4-(8-Benzo[b]thiophen-2-yl-7-fluoro-imidazo[4,5-c]quinolin-1-yl)-phenyl]-acetonitrile, analytical HPLC: t_{R}=8.75 minutes (Grad 1); ES⁺-MS: m/eₒ=435.0.

### Example 60

### 2-[4-(8-Benzo[1,3]dioxol-5-yl-7-fluoro-imidazo[4,5-c]quinolin-1-yl)-phenyl]-ethylamine

2-[4-(8-Benzo[1,3]dioxol-5-yl-7-fluoro-imidazo[4,5-c]quinolin-1-yl)phenyl]-ethylamine is obtained as described in Example 4 using [4-(8-benzo[1,3]dioxol-5-yl-7-fluoro-imidazo[4,5-c]quinolin-1-yl)-phenyl]-acetonitrile (Example 53) as starting material. 2-[4-(8-Benzo[1,3]dioxol-5-yl-7-fluoro-imidazo[4,5-c]quinolin-1-yl)-phenyl]-ethylamine, analytical HPLC: t_{R}=5.72 minutes (Grad 1); ES⁺-MS: m/eₒ=427.2.

### Example 61

### 2-[4-(7-Fluoro-8-thiophen-2-yl-imidazo[4,5-c]quinolin-1-yl)-phenyl]-ethylamine

2-[4-(7-Fluoro-8-thiophen-2-yl-imidazo[4,5-c]quinolin-1-yl)-phenyl]-ethylamine is obtained as described in Example 4 using [4-(7-fluoro-8-thiophen-2-yl-imidazo[4,5-c]quinolin-1-yl)-phenyl]-acetonitrile (Example 55) as starting material. 2-[4-(7-Fluoro-8-thiophen-2-yl-imidazo[4,5-c]quinolin-1-yl)-phenyl]-ethylamine, analytical HPLC: t_{R}=5.62 minutes (Grad 1); ES⁺-MS: m/eₒ=389.2.

### Example 62

### 2-[4-(7-Fluoro-8-thiophen-3-yl-imidazo[4,5-c]quinolin-1-yl)-phenyl]-ethylamine

2-[4-(7-Fluoro-8-thiophen-3-yl-imidazo[4,5-c]quinolin-1-yl)-phenyl]-ethylamine is obtained as described in Example 4 using [4-(7-fluoro-8-thiophen-3-yl-imidazo[4,5-c]quinolin-1-yl)-phenyl]-acetonitrile (Example 56) as starting material. 2-[4-(7-Fluoro-8-thiophen-3-yl-imidazo[4,5c]quinolin-1-yl)-phenyl]-ethylamine, analytical HPLC: t_{R}=5.73 minutes (Grad 1); ES⁺-MS: m/e, =389.3.

### Example 63

### 2-[4-(8-Benzofuran-2-yl-7-fluoro-imidazo[4,5-c]quinolin-1-yl)-phenyl]-ethylamine

2-[4-(8-Benzofuran-2-yl-7-fluoro-imidazo[4,5-c]quinolin-1-yl)-phenyl]-ethylamine is obtained as described in Example 4 using [4-(8-benzofuran-2-yl-7-fluoro-imidazo[4,5-c]quinolin-1-yl)-phenyl]-acetonitrile (Example 57) as starting material. 2-[4-(8-Benzofuran-2-yl-7-fluoro-imidazo[4,5-c]quinolin-1-yl)-phenyl]-ethylamine, analytical HPLC: t_{R}=6.35 minutes (Grad 1); ES⁺-MS: m/eₒ=423.3.

### Example 64

### 2-{4-[7-Fluoro-8-(1H-indol-5-yl)-imidazo[4,5-c]quinolin-1-yl]-phenyl}-ethylamine

2-{4-[7-Fluoro-8-(1H-indol-5-yl)-imidazo[4,5-c]quinolin-1-yl]-phenyl}-ethylamine is obtained as described in Example 4 using {4-[7-fluoro-8-(1H-indol-5-yl)-imidazo[4,5c]quinolin-1-yl]-phenyl}-acetonitrile (Example 58) as starting material. 2-{4-[7-Fluoro-8-(1H-indol-5-yl)-imidazo[4,5-c]quinolin-1-yl]-phenyl}-ethylamine, analytical HPLC: t_{R}=5.60 minutes (Grad 1); ES⁺-MS: m/eₒ=422.3.

### Example 65

### 2-[4-(8-Benzo[b]thiophen-2-yl-7-fluoro-imidazo[4,5-c]quinolin-1-yl)-phenyl]-ethylamine

2-[4-(8-Benzo[b]thiophen-2-yl-7-fluoro-imidazo[4,5-c]quinolin-1-yl)-phenyl]-ethylamine is obtained as described in Example 4 using [4-(8-benzo[b]thiophen-2-yl-7-fluoro-imidazo[4,5-c]quinolin-1-yl)-phenyl]-acetonitrile (Example 59) as starting material. 2-[4-(8-Benzo[b]thiophen-2-yl-7-fluoro-imidazo[4,5-c]quinolin-1-yl)-phenyl]-ethylamine, analytical HPLC: t_{R}=6.29 minutes (Grad 1); ES⁺-MS: m/eₒ=439.2.

### Example 66

### 3-[4-(8-Benzo[1,3]dioxol-5-yl-7-fluoro-imidazo[4,5-c]quinolin-1-yl)-phenyl]-propionitrile

3-[4-(8-Benzo[1,3]dioxol-5-yl-7-fluoro-imidazo[4,5-c]quinolin-1-yl)phenyl]-propionitrile is synthesized as described in Example 1 using 6-bromo-4 chloro-7-fluoro-3-nitro-quinoline (Example 54a) and 3-(4-amino-phenyl)-propionitrile (Example 31a). 3-[4-(8-Benzo[1,3]dioxol-5-yl-7-fluoro-imidazo[4,5-c]quinolin-1-yl)-phenyl]-propionitrile, analytical HPLC: t_{R}=6.93 minutes (Grad 1); ES⁺-MS: m/eₒ=437.1.

### Example 67

### 3-[4-(7-Fluoro-8-thiophen-2-yl-imidazo[4,5-c]quinolin-1-yl)-phenyl]-propionitrile

3-[4-(7-Fluoro-8-thiophen-2-yl-imidazo[4,5-c]quinolin-1-yl)-phenyl]-propionitrile is synthesized as described in Example 1 using 6-bromo-4-chloro-7-fluoro-3-nitro-quinoline (Example 54a), 3-(4-amino-phenyl)-propionitrile (Example 31a) and thiophene-2-boronic acid. 3-[4-(7-Fluoro-8-thiophen-2-yl-imidazo[4,5-c]quinolin-1-yl)-phenyl]-propionitrile, analytical HPLC: t_{R}=7.30 minutes (Grad 1); ES⁺-MS: m/eₒ=399.1.

### Example 68

### 3-[4-(7-Fluoro-8-thiophen-3-yl-imidazo[4,5-c]quinolin-1-yl)-phenyl]-propionitrile

3-[4-(7-Fluoro-8-thiophen-3-yl-imidazo[4,5-c]quinolin-1-yl)-phenyl]-propionitrile is obtained as described in Example 1 using 6-bromo-4-chloro-7-fluoro-3-nitro-quinoline (Example 54a), 3-(4-amino-phenyl)-propionitrile (Example 31a) and thiophene-3-boronic acid. 3-[4-(7-Fluoro-8-thiophen-3-yl-imidazo[4,5-c]quinolin-1-yl)-phenyl]-propionitrile, analytical HPLC: t_{R}=7.00 minutes (Grad 1); ES⁺-MS: m/eₒ=399.1.

### Example 69

### 3-[4-(8-Benzofuran-2-yl-7-fluoro-imidazo[4,5-c]quinolin-1-yl)-phenyl]-propionitrile

3-[4-(8-Benzofuran-2-yl-7-fluoro-imidazo[4,5-c]quinolin-1-yl)-phenyl]-propionitrile is obtained as described in Example 1 using 6-bromo-4-chloro-7-fluoro-3-nitro-quinoline (Example 54a), 3-(4-amino-phenyl)-propionitrile (Example 31a) and benzo[b]furan-2-boronic acid. 3-[4-(8-Benzofuran-2-yl-7-fluoro-imidazo[4,5-c]quinolin-1-yl)-phenyl]-propionitrile, analytical HPLC: t_{R}=8.38 minutes (Grad 1); ES⁺-MS: m/eₒ=433.0.

### Example 70

### 3-[4-(8-Benzo[b]thiophen-2-yl-7-fluoro-imidazo[4,5-c]quinolin-1-yl)-phenyl]-propionitrile

3-[4-(8-Benzo[b]thiophen-2-yl-7-fluoro-imidazo[4,5-c]quinolin-1-yl)-phenyl]-propionitrile is obtained as described in Example 1 using 6-bromo-4 chloro-7-fluoro-3-nitro-quinoline (Example 54a), 3-(4-amino-phenyl)-propionitrile (Example 31a) and benzo[b]thiophene-2-boronic acid. 3-[4-(8-Benzo[b]thiophen-2-yl-7-fluoro-imidazo[4,5-c]quinolin-1-yl)-phenyl]-propionitrile, analytical HPLC: t_{R}=8.58 minutes (Grad 1); ES⁺-MS: m/eₒ=449.1.

### Example 71

### 3-{4-[7-Fluoro-8-(H-indol-5-yl)-imidazo[4,5-c]quinolin-1-yl]-phenyl}-propionitrile

3-{4-[7-Fluoro-8-(1H-indol-5-yl)-imidazo[4,5-c]quinolin-1-yl]-phenyl}-propionitrile is obtained as described in Example 1 using 6-bromo-4-chloro-7-fluoro-3-nitro-quinoline (Example 54a), 3-(4-amino-phenyl)-propionitrile (Example 31a) and 5-indolylboronic acid. 3-{4-[7-Fluoro-8-(1H-indol-5-yl)-imidazo[4,5-c]quinolin-1-yl]-phenyl}-propionitrile, analytical HPLC: t_{R}=6.87 minutes (Grad 1); ES⁺-MS: m/eₒ=432.1.

### Example 72

### 3-[4-(8-Benzo[1,3]dioxol-5-yl-7-fluoro-imidazo[4,5-c]quinolin-1-yl)-phenyl]-propylamine

3-[4-(8-Benzo[1,3]dioxol-5-yl-7-fluoro-imidazo[4,5-c]quinolin-1-yl)-phenyl]-propylamine is obtained as described in Example 4 using 3-[4-(8-benzo[1,3]dioxol-5-yl-7-fluoro-imidazo[4,5-c]quinolin-1-yl)-phenyl]-propionitrile (Example 66) as starting material. 3-[4-(8-Benzo[1,3]dioxol-5-yl-7-fluoro-imidazo[4,5-c]quinolin-1-yl)-phenyl]-propylamine, analytical HPLC: t_{R}=6.36 minutes (Grad 1); ES⁺-MS: m/eₒ=441.5.

### Example 73

### 3-[4-(7-Fluoro-8-thiophen-2-yl-imidazo[4,5-c]quinolin-1-yl)-phenyl]-propylamine

3-[4-(7-Fluoro-4-thiophen-2-yl-imidazo[4,5-c]quinolin-1-yl)-phenyl]-propylamine is obtained as described in Example 4 using 3-[4-(7-fluoro-8-thiophen-2-yl-imidazo[4,5-c]quinolin-1-yl)-phenyl]-propionitrile (Example 67) as starting material. 3-[4-(7-Fluoro-thiophen-2-yl-imidazo[4,5-c]quinolin-1-yl)-phenyl]-propylamine, analytical HPLC: t_{R}=5.82 minutes (Grad 1); ES⁺-MS: m/eₒ=403.1.

### Example 74

### 3-[4-(7-Fluoro-8-thiophen-3-yl-imidazo[4,5-c]quinolin-1-yl)-phenyl]-propylamine

3-[4-(7-Fluoro-8-thiophen-3-yl-imidazo[4,5-c]quinolin-1-yl)-phenyl]-propylamine is obtained as described in Example 4 using 3-[4-(7-fluoro-8-thiophen-3-yl-imidazo[4,5-c]quinolin-1-yl)phenyl]-propionitrile (Example 68) as starting material. 3-[4-(7-Fluoro-8-thiophen-3-yl-imidazo[4,5-c]quinolin-1-yl)-phenyl]-propylamine, analytical HPLC: t_{R}=5.71 minutes (Grad 1); ES⁺-MS: m/eₒ=403.3.

### Example 75

### 3-[4-(8-Benzofuran-2-yl-7-fluoro-imidazo[4,5-c]quinolin-1-yl)-phenyl]-propylamine

3-[4-(8-Benzofuran-2-yl-7-fluoro-imidazo[4,5-c]quinolin-1-yl)-phenyl]-propylamine is obtained as described in Example 4 using 3-[4-(8-benzofuran-2-yl-7-fluoro-imidazo[4,5-c]quinolin-1-yl)-phenyl]-propionitrile (Example 69) as starting material. 3-[4-(8-Benzofuran-2-yl-7-fluoro-imidazo[4,5-c]quinolin-1-yl)-phenyl]-propylamine, analytical HPLC: t_{R}=6.38 minutes (Grad 1); ES⁺-MS: m/eₒ=437.3.

### Example 76

### 3-[4-(8-Benzo[b]thiophen-2-yl-7-fluoro-imidazo[4,5-c]quinolin-1-yl)-phenyl]-propylamine

3-[4-(8-Benzo[b]thiophen-2-yl-7-fluoro-imidazo[4,5-c]quinolin-1-yl)-phenyl]-propylamine is obtained as described in Example 4 using 3-[4-(8-benzo[b]thiophen-2-yl-7-fluoro-imidazo[4,5-c]quinolin-1-yl)-phenyl]-propionitrile (Example 70) as starting material. 3-[4-(8-Benzo[b]thiophen-2-yl-7-fluoro-imidazo[4,5-c]quinolin-1-yl)-phenyl]-propylamine, analytical HPLC: t_{R}=6.51 minutes (Grad 1); ES⁺-MS: m/eₒ=453.3.

### Example 77

### 3-{4-[7-Fluoro-8-(1H-indol-5-yl)-imidazo[4,5-c]quinolin-1-yl]-phenyl}-propylamine

3-{4-[7-Fluoro-8-(1H-indol-5-yl)-imidazo[4,5-c]quinolin-1-yl]-phenyl}-propylamine is obtained as described in Example 4 using 3-{4-[7-fluoro-8-(1H-indol-5-yl)-imidazo[4,5-c]quinolin-1-yl]-phenyl}-propionitrile (Example 71) as starting material. 3-(4-[7-Fluoro-8-(1H-indol-5-yl) imidazo[4,5-c]quinolin-1-yl]-phenyl)propylamine, analytical HPLC: t_{R}=5.82 minutes (Grad 1); ES⁺-MS: m/eₒ=436.2.

### Example 78

### [3-Chloro-4-(7-fluoro-8-thiophen-2-yl-imidazo[4,5-c]quinolin-1-yl)-phenyl]-acetonitrile

[3-Chloro-4-(7-fluoro-8-thiophen-2-yl-imidazo[4,5-c]quinolin-1-yl)-phenyl]-acetonitrile is obtained as described in Example 1 using 6-bromo-4-chloro-7-fluoro-3-nitro-quinoline (Example 54a), (4-amino-3-chloro-phenyl)-acetonitrile (Example 7a) and thiophene-2-boronic acid. [3-Chloro-4-(7-fluoro-8-thiophen-2-yl-imidazo[4,5-c]quinolin-1-yl)-phenyl]-acetonitrile, analytical HPLC: t_{R}=7.50 minutes (Grad 1); ES⁺-MS: m/eₒ=419.3.

### Example 79

### [3-Chloro-4-(7-fluoro-8-thiophen-3-yl-imidazo[4,5-c]quinolin-1-yl)-phenyl]-acetonitrile

[3-Chloro-4-(7-fluoro-8-thiophen-3-yl-imidazo[4,5-c]quinolin-1-yl)-phenyl]-acetonitrile is obtained as described in Example 1 using 6-bromo-4-chloro-7-fluoro-3-nitro-quinoline (Example 54a), (4-amino-3-chloro-phenyl)-acetonitrile (Example 7a) and thiophene-3-boronic acid. [3-Chloro-4-(7-fluoro-8-thiophen-3-yl-imidazo[4,5-c]quinolin-1-yl)-phenyl]-acetonitrile, analytical HPLC: t_{R}=7.35 minutes (Grad 1); ES⁺-MS: m/eₒ=419.2.

### Example 80

### {3-Chloro-4-[7-fluoro-8-(1H-indol-5-yl)-imidazo[4,5-c]quinolin-1-yl]-phenyl}-acetonitrile

{3-Chloro-[7-fluoro-8-(1H-indol-5-yl)-imidazo[4,5-c]quinolin-1-yl]-phenyl}-acetonitrile is obtained as described in Example 1 using 6-bromo-4-chloro-7-fluoro-3-nitro-quinoline (Example 54a), (4-amino-3-chloro-phenyl)-acetonitrile (Example 7a) and 5-indolylboronic acid. {3-Chloro-4-[7-fluoro-8-(1H-indol-5-yl)-imidazo[4,5-c]quinolin-1-yl]-phenyl}-acetonitrile, analytical HPLC: t_{R}=7.03 minutes (Grad 1); ES⁺-MS: m/eₒ=452.3.

### Example 81

### 2-[3-Chloro-4-(7-fluoro-8-thiophen-2-yl-imidazo[4,5-c]quinolin-1-yl)-phenyl]-ethylamine

2-[3-Chloro-4-(7-fluoro-8-thiophen-2-yl-imidazo[4,5-c]quinolin-1-yl)-phenyl]-ethylamine is obtained as described in Example 4 using [3-chloro-4-(7-fluoro-8-thiophen-2-yl-imidazo[4,5-c]quinolin-1-yl)-phenyl]-acetonitrile (Example 78) as starting material. 2-[3-Chloro-4-(7-fluoro-8-thiophen-2-yl-imidazo[4,5-c]quinolin-1-yl)-phenyl]-ethylamine, analytical HPLC: t_{R}=6.01 minutes (Grad 1); ES⁺-MS: m/eₒ=423.3.

### Example 82

### 2-[3-Chloro-4-(7-fluoro-8-thiophen-3-yl-imidazo[4,5-c]quinolin-1-yl)-phenyl]-ethylamine

2-[3-Chloro-4-(7-fluoro-8-thiophen-3-yl-imidazo[4,5-c]quinolin-1-yl)-phenyl]-ethylamine is obtained as described in Example 4 using [3-chloro-4-(7-fluoro-8-thiophen-3-yl-imidazo[4,5-c]quinolin-1-yl)-phenyl]-acetonitrile (Example 79) as starting material. 2-[3-Chloro-4-(7-fluoro-8-thiophen-3-yl-imidazo[4,5-c]quinolin-1-yl)-phenyl]-ethylamine, analytical HPLC: t_{R}=6.01 minutes (Grad 2); ES⁺-MS: m/eₒ=423.3.

### Example 83

### 2-{3-Chloro-4-[7-fluoro-8-(1H-indol-5-yl)-imidazo[4,5-c]quinolin-1-yl]-phenyl}-ethylamine

2-{3-Chloro-4-[7-fluoro-8-(1H-indol-5-yl)-imidazo[4,5-c]quinolin-1-yl]-phenyl}ethylamine is obtained as described in Example 4 using {3-chloro-4-[7-fluoro-8-(1H-indol-5-yl)-imidazo[4,5-c]quinolin-1-yl]-phenyl}-acetonitrile (Example 80) as starting material. 2-{3-Chloro-4-[7-fluoro-8-(1H-indol-5-yl)-imidazo[4,5-c]quinolin-1-yl]-phenyl}ethylamine, analytical HPLC: t_{R}=6.03 minutes (Grad 1); ES⁺-MS: m/eₒ=456.4.

### Example 84

### [4-(7-Chloro-8-thiophen-2-yl-imidazo[4,5-c]quinolin-1-yl)-phenyl]-acetonitrile

[4-(7-Chloro-8-thiophen-2-yl-imidazo[4,5-c]quinolin-1-yl)-phenyl]-acetonitrile is obtained as described in Example 1 using 6-bromo-4,7-dichloro-3-nitro-quinoline (Example 84a) and thiophene-2-boronic acid. [4-(7-Chloro-8-thiophen-2-yl-imidazo[4,5-c]quinolin-1-yl)-phenyl]-acetonitrile, analytical HPLC: t_{R}=7.44 minutes (Grad 1); ES⁺-MS: m/eₒ=401.1.

### Example 84a

### 6-Bromo-4,7-dichloro-3-nitro-quinoline

6-Bromo-4,7-dichloro-3-nitro-quinoline is obtained as described in analogy to 6-bromo-4-chloro-3-nitro-quinoline (Example 1c) starting from 2-amino-5-bromo-4-chloro-benzoic acid (Example 84b). 6-Bromo-4,7-dichloro-3-nitro-quinoline, analytical HPLC: t_{R}=9.48 minutes

(Grad 1); ES⁺-MS: m/eₒ=323.0.

### Example 84b

### 2-Amino-5-bromo-4-chloro-benzoic acid

### 2-Amino-5-bromo-4-chloro-benzoic acid is obtained as described in Example 54b starting with 2-amino-4-chlorobenzoic acid. 2-Amino-5-bromo-4-chloro-benzoic acid, analytical HPLC: t_{R}=3.21 minutes (Grad 5).

¹H NMR (DMSO-d₆): δ 7.85 (s, 1H), 6.95 (s, 1H).

### Example 85

### [4-(7-Chloro-8-thiophen-3-yl-imidazo[4,5-c]quinolin-1-yl)-phenyl]-acetonitrile

[4-(7-Chloro-8-thiophen-3-yl-imidazo[4,5-c]quinolin-1-yl)-phenyl]-acetonitrile is obtained as described in Example 1 using 6-bromo-4,7-dichloro-3-nitro-quinoline (Example 84a) and thiophene-3-boronic acid. [4-(7-Chloro-8-thiophen-3-yl-imidazo[4,5-c]quinolin-1-yl)-phenyl]-acetonitrile, analytical HPLC: t_{R}=7.23 minutes (Grad 1); ES⁺-MS: m/eₒ=401.1.

### Example 86

### [4-(8-Benzofuran-2-yl-7-chloro-imidazo[4,5-c]quinolin-1-yl)-phenyl]-acetonitrile

[4-(8-Benzofuran-2-yl-7-chloro-imidazo[4,5-c]quinolin-1-yl)-phenyl]-acetonitrile is obtained as described in Example 1 using 6-bromo-4,7-dichloro-3-nitro-quinoline (Example 84a) and benzo[b]furan-2-boronic acid. [4-(8-Benzofuran-2-yl-7-chloro-imidazo[4,5-c]quinolin-1-yl)-phenyl]-acetonitrile, analytical HPLC: t_{R}=7.44 minutes (Grad 1); ES⁺-MS: m/eₒ=401.1.

### Example 87

### {4-[7-Chloro-8-(1H-indol-5-yl)-imidazo[4,5-c]quinolin-1-yl]-phenyl}acetonitrile

{4-[7-Chloro-8-(1H-indol-5-yl)-imidazo[4,5-c]quinolin-1-yl]-phenyl}-acetonitrile is obtained as described in Example 1 using 6-bromo-4,7-dichloro-3-nitro-quinoline (Example 84a) and 5-indolylboronic acid. {4-[7-Chloro-8-(1H-indol-5-yl)-imidazo[4,5-c]quinolin-1-yl]-phenyl}acetonitrile, analytical HPLC: t_{R}=6.93 minutes (Grad 1); ES⁺-MS: m/eₒ=434.1.

### Example 88

### [4-(8-Benzo[b]thiophen-2-yl-7-chloro-imidazo[4,5-c]quinolin-1-yl)-phenyl]-acetonitrile

[4-(8-Benzo[b]thiophen-2-yl-7-chloro-imidazo[4,5c]quinolin-1-yl)-phenyl]-acetonitrile is obtained as described in Example 1 using 6-bromo-4,7-dichloro-3-nitro-quinoline (Example 84a) and benzo[b]thiophene-2-boronic acid. [4-(8-Benzo[b]thiophen-2-yl-7-chloro-imidazo[4,5-c]quinolin-1-yl)-phenyl]-acetonitrile, analytical HPLC: t_{R}=8.83 minutes (Grad 1); ES⁺-MS: m/eₒ=451.1.

### Example 89

### 2-[4-(7-Chloro-8-thiophen-2-yl-imidazo[4,5-c]quinolin-1-yl)-phenyl]-ethylamine

2-[4-(7-Chloro-8-thiophen-2-yl-imidazo[4,5-c]quinolin-1-yl)-phenyl]-ethylamine is obtained as described in Example 4 using [4-(7-chloro-8-thiophen-2-yl-imidazo[4,5-c]quinolin-1-yl)-phenyl]-acetonitrile (Example 84) as starting material. 2-[4-(7-Chloro-8-thiophen-2-yl-imidazo[4,5-c]quinolin-1-yl)-phenyl]-ethylamine, analytical HPLC: t_{R}=5.91 minutes (Grad 1); ES⁺-MS: m/eₒ=405.2.

### Example 90

### 2-[4-(7-Chloro-8-thiophen-3-yl-imidazo[4,5-c]quinolin-1-yl)-phenyl]-ethylamine

2-[4-(7-Chloro-8-thiophen-3-yl-imidazo[4,5-c]quinolin-1-yl)-phenyl]-ethylamine is obtained as described in Example 4 using [4-(7-chloro-8-thiophen-3-yl-imidazo[4,5-c]quinolin-1-yl)-phenyl]-acetonitrile (Example 85) as starting material. 2-[4-(7-Chloro-8-thiophen-3-yl-imidazo[4,5-c]quinolin-1-yl)-phenyl]-ethylamine, analytical HPLC: t_{R}=5.87 minutes (Grad 1); ES⁺-MS: m/eₒ=405.2.

### Example 91

### 2-[4-(8-Benzofuran-2-yl-7-chloro-imidazo[4,5-c]quinolin-1-yl)-phenyl]-ethylamine

2-[4-(8-Benzofuran-2-yl-7-chloro-imidazo[4,5-c]quinolin-1-yl)-phenyl]-ethylamine is obtained as described in Example 4 using [4-(8-benzofuran-2-yl-7-chloro-imidazo[4,5-c]quinolin-1-yl)-phenyl]-acetonitrile (Example 86) as starting material. 2-[4-(8-Benzofuran-2-yl-7-chloro-imidazo[4,5-c]quinolin-1-yl)-phenyl]-ethylamine, analytical HPLC: t_{R}=6.52 minutes (Grad 1); ES⁺-MS: m/eₒ=439.2.

### Example 92

### 2-{4-[7-Chloro-8-(1H-indol-5-yl)-imidazo[4,5-c]quinolin-1-yl]-phenyl}-ethylamine

2-{4-[7-Chloro-8-(1H-indol-5-yl)-imidazo[4,5-c]quinolin-1-yl]-phenyl}-ethylamine is obtained as described in Example 4 using (4-[7-chloro-8-(1H-indol-5-yl)-imidazo[4,5-c]quinolin-1-yl]-phenyl)acetonitrile (Example 87) as starting material. 2-{4-[7-Chloro-8-(1H-indol-5-yl)-imidazo[4,5-c]quinolin-1-yl]-phenyl}-ethylamine, analytical HPLC: t_{R}=5.83 minutes (Grad 1); ES⁺-MS: m/eₒ=438.2.

### Example 93

### 2-[4-(8-Benzo[b]thiophen-2-yl-7-chloro-imidazo[4,5-c]quinolin-1-yl)-phenyl]-ethylamine

2-[4-(8-Benzo[b]thiophen-2-yl-7-chloro-imidazo[4,5-c]quinolin-1-yl)-phenyl]-ethylamine is obtained as described in Example 4 using [4-(8-benzo[b]thiophen-2-yl-7-chloro-imidazo[4,5-c]quinolin-1-yl)-phenyl]-acetonitrile (Example 88) as starting material. 2-[4-(8-Benzo[b]thiophen-2-yl-7-chloro-imidazo[4,5-c]quinolin-1-yl)-phenyl]-ethylamine, analytical HPLC: t_{R}=6.55 minutes (Grad 1); ES⁺-MS: m/eₒ=455.3.

### Example 94

### 3-[4-(8-Benzo[1,3]dioxol-5-yl-7-chloro-imidazo[4,5-c]quinolin-1-yl)-phenyl]-propionitrile

3-[4-(8-Benzo[1,3]dioxol-5-yl-7-chloro-imidazo[4,5-c]quinolin-1-yl)-phenyl]-propionitrile is obtained as described in Example 4 using 6-bromo-4,7-dichloro-3-nitro-quinoline (Example 84a) and 3-(4-amino-phenyl)-propionitrile (Example 31a). 3-[4-(8-Benzo[1,3]dioxol-5-yl-7-chloro-imidazo[4,5c]quinolin-1-yl)-phenyl]-propionitrile, analytical HPLC: t_{R}=7.34 minutes (Grad 1); ES⁺-MS: m/eₒ=453.1.

### Example 95

### 3-[4-(7-Chloro-8-thiophen-2-yl-imidazo[4,5-c]quinolin-1-yl)-phenyl]-propionitrile

3-[4-(7-Chloro-8-thiophen-2-yl-imidazo[4,5-c]quinolin-1-yl)-phenyl]-propionitrile is obtained as described in Example 1 using 6-bromo-4,7-dichloro-3-nitro-quinoline (Example 84a), 3-(4-amino-phenyl)-propionitrile (Example 31a) and thiophene-2-boronic acid. 3-[4-(7-Chloro-8-thiophen-2-yl-imidazo[4,5-c]quinolin-1-yl)-phenyl]-propionitrile, analytical HPLC: t_{R}=7.54 minutes (Grad 1); ES⁺-MS: m/eₒ=415.1.

### Example 96

### 3-[4-(8-Benzofuran-2-yl-7-chloro-imidazo[4,5-c]quinolin-1-yl)-phenyl]-propionitrile

3-[4-(8-Benzofuran-2-yl-7-chloro-imidazo[4,5-c]quinolin-1-yl)-phenyl]-propionitrile is obtained as described in Example 1 using 6-bromo-4,7-dichloro-3-nitro-quinoline (Example 84a), 3-(4-amino-phenyl)-propionitrile (Example 31a) and benzo[b]furan-2-boronic acid. 3-[4-(8-Benzofuran-2-yl-7-chloro-imidazo[4,5-c]quinolin-1-yl)-phenyl]-propionitrile, analytical HPLC: t_{R}=8.93 minutes (Grad 1); ES⁺-MS: m/eₒ=449.1.

### Example 97

### 3-{4-[7-Chloro-8-(1H-indol-5-yl)-imidazo[4,5-c]quinolin-1-yl]-phenyl}-propionitrile

3-{4-[7-Chloro-8-(1H-indol-5-yl)-imidazo[4,5-c]quinolin-1-yl]-phenyl}-propionitrile is obtained as described in Example 1 using 6-bromo-4,7-dichloro-3-nitro-quinoline (Example 84a), 3-(4-amino-phenyl)-propionitrile (Example 31a) and 5-indolylboronic acid. 3-{4-[7-Chloro-8-(1H-indol-5-yl)-imidazo[4,5-c]quinolin-1-yl]-phenyl}-propionitrile, analytical HPLC: t_{R}=7.01 minutes (Grad 1); ES⁺-MS: m/eₒ=448.1.

### Example 98

### 3-[4-(8-Benzo[b]thiophen-2-yl-7-chloro-imidazo[4,5-c]quinolin-1-yl)-phenyl]-propionitrile

3-[4-(8-Benzo[b]thiophen-2-yl-7-chloro-imidazo[4,5-c]quinolin-1-yl)-phenyl]-propionitrile is obtained as described in Example 1 using 6-bromo-4,7-dichloro-3-nitro-quinoline (Example 84a), 3-(4-amino-phenyl)-propionitrile (Example 31a) and benzo[b]thiophene-2-boronic acid. 3-[4-(8-Benzo[b]thiophen-2-yl-7-chloro-imidazo[4,5-c]quinolin-1-yl)-phenyl]-propionitrile, analytical HPLC: t_{R}=8.74 minutes (Grad 1); ES⁺-MS: m/eₒ=465.0.

### Example 99

### 3-[4-(8-Benzo[1,3]dioxol-5-yl-7-chloro-imidazo[4,5-c]quinolin-1-yl)-phenyl]-propylamine

3-[4-(8-Benzo[1,3]dioxol-5-yl-7-chloro-imidazo[4,5-c]quinolin-1-yl)-phenyl]-propylamine is obtained as described in Example 4 using 3-[4-(8-benzo[1,3]dioxol-5-yl-7-chloro-imidazo[4,5-c]quinolin-1-yl)-phenyl]-propionitrile (Example 94) as starting material. 3-[4-(8-Benzo[1,3]dioxol-5-yl-7-chloro-imidazo[4,5-c]quinolin-1-yl)-phenyl]-propylamine, analytical HPLC: t_{R}=5.98 minutes (Grad 1); ES⁺-MS: m/eₒ=457.1.

### Example 100

### 3-[4-(7-Chloro-8-thiophen-2-yl-imidazo[4,5-c]quinolin-1-yl)-phenyl]-propylamine

3-[4-(7-Chloro-8-thiophen-2-yl-imidazo[4,5-c]quinolin-1-yl)-phenyl]-propylamine is obtained as described in Example 4 using 3-[4-(7-chloro-8-thiophen-2-yl-imidazo[4,5-c]quinolin-1-yl)phenyl]-propionitrile (Example 95) as starting material. 3-[4-(7-Chloro-8-thiophen-2-yl-imidazo[4,5-c]quinolin-1-yl)-phenyl]-propylamine, analytical HPLC: t_{R}=6.11 minutes (Grad 1); ES⁺-MS: m/eₒ=419.3.

### Example 101

### 3-[4-(8-Benzofuran-2-yl-7-chloro-imidazo[4,5-c]quinolin-1-yl)-phenyl]-propylamine

3-[4-(8-Benzofuran-2-yl-7-chloro-imidazo[4,5-c]quinolin-1-yl)-phenyl]-propylamine is obtained as described in Example 4 using 3-[4-(8-benzofuran-2-yl-7-chloro-imidazo[4,5-c]quinolin-1-yl)-phenyl]-propionitrile (Example 96) as starting material. 3-[4-(8-Benzofuran-2-yl-7-chloro-imidazo[4,5-c]quinolin-1-yl)-phenyl]-propylamine, analytical HPLC: t_{R}=6.62 minutes (Grad 1); ES⁺-MS: m/eₒ=453.4.

### Example 102

### 3-{4-[7-Chloro-8-(1H-indol-5-yl)-imidazo[4,5-c]quinolin-1-yl]-phenyl}-propylamine

3-[4-(7-Chloro-8-(1H-indol-5-yl)imidazo[4,5-c]quinolin-1-yl]-phenyl)propylamine is obtained as described in Example 4 using 3-{4-[7-chloro-8-(1H-indol-5-yl)-imidazo[4,5-c]quinolin-1-yl]-phenyl}-propionitrile (Example 97) as starting material. 3-{4-[7-Chloro-8-(1H-indol-5-yl)-imidazo[4,5-c]quinolin-1-yl]-phenyl}-propylamine, analytical HPLC: t_{R}=6.03 minutes (Grad 1); ES⁺-MS: m/eₒ=452.3.

### Example 103

### 3-[4-(8-Benzo[b]thiophen-2-yl-7-chloro-imidazo[4,5-c]quinolin-1-yl)-phenyl]-propylamine

3-[4-(8-Benzo[b]thiophen-2-yl-7-chloro-imidazo[4,5-c]quinolin-1-yl)-phenyl]-propylamine is obtained as described in Example 4 using 3-[4-(8-benzo[b]thiophen-2-yl-7-chloro-imidazo[4,5-c]quinolin-1-yl)-phenyl]-propionitrile (Example 98) as starting material. 3-[4-(8-Benzo[b]thiophen-2-yl-7-chloro-imidazo[4,5-c]quinolin-1-yl)-phenyl]-propylamine, analytical HPLC: t_{R}=6.62 minutes (Grad 1); ES⁺-MS: m/eₒ=469.3.

### Example 104

### [3-Chloro-4-(7-chloro-8-thiophen-2-yl-imidazo[4,5-c]quinolin-1-yl)-phenyl]-acetonitrile

[3-Chloro-4-(7-chloro-8-thiophen-2-yl-imidazo[4,5-c]quinolin-1-yl)-phenyl]-acetonitrile is obtained as described in Example 1 using 6-bromo-4,7-dichloro-3-nitro-quinoline (Example 84a), (4-amino-3-chloro-phenyl)-acetonitrile (Example 7a) and thiophene-2-boronic acid. [3-Chloro-4-(7-chloro-8-thiophen-2-yl-imidazo[4,5-c]quinolin-1-yl)-phenyl]-acetonitrile, analytical HPLC: t_{R}=7.93 minutes (Grad 1); ES⁺-MS: m/eₒ=434.9.

### Example 105

### [3-Chloro-4-(7-chloro-8-thiophen-3-yl-Imidazo[4,5-c]quinolin-1-yl)-phenyl]-acetonitrile

[3-Chloro-4-(7-chloro-8-thiophen-3-yl-imidazo[4,5-c]quinolin-1-yl)-phenyl]-acetonitrile is obtained as described in Example 1 using 6-bromo-4,7-dichloro-3-nitro-quinoline (Example 84a), (4-amino-3-chloro-phenyl)-acetonitrile (Example 7a) and thiophene-3-boronic acid. [3-Chloro-4-(7-chloro-8-thiophen-3-yl-imidazo[4,5-c]quinolin-1-yl)-phenyl]-acetonitrile, analytical HPLC: t_{R}=7.51 minutes (Grad 1); ES⁺-MS: m/eₒ=435.0.

### Example 106

### 2-[3-Chloro-4-(7-chloro-8-thiophen-2-yl-imidazo[4,5-c]quinolin-1-yl)-phenyl]-ethylamine

2-[3-Chloro-4-(7-chloro-8-thiophen-2-yl-imidazo[4,5-c]quinolin-1-yl)-phenyl]-ethylamine is obtained as described in Example 4 using [3-chloro-4-(7-chloro-8-thiophen-2-yl-imidazo[4,5-c]quinolin-1-yl)-phenyl]-acetonitrile (Example 104). 2-[3-Chloro-4-(7-chloro-8-thiophen-2-yl-imidazo[4,5-c]quinolin-1-yl)-phenyl]-ethylamine, analytical HPLC: t_{R}=6.16 minutes (Grad 1); ES⁺-MS: m/eₒ=439.0.

### Example 107

### 2-[3-Chloro-4-(7-chloro-8-thiophen-3-yl-imidazo[4,5-c]quinolin-1-yl)-phenyl]-ethylamine

2-[3-Chloro-4-(7-chloro-8-thiophen-3-yl-imidazo[4,5-c]quinolin-1-yl)-phenyl]-ethylamine is obtained as described in Example 4 using [3-chloro-4-(7-chloro-8-thiophen-3-yl-imidazo[4,5-c]quinolin-1-yl)-phenyl]-acetonitrile (Example 105) as starting material. 2-[3-Chloro-4-(7-chloro-8-thiophen-3-yl-imidazo[4,5-c]quinolin-1-yl)-phenyl]-ethylamine, analytical HPLC: t_{R}=6.12 minutes (Grad 1); ES⁺-MS: m/eₒ=438.9.

### Example 108

### 3-[3-Chloro-4-(7-chloro-8-thiophen-2-yl-imidazo[4,5]-quinolin-1-yl)-phenyl]-propionitrile

3-[3-Chloro-4-(7-chloro-8-thiophen-2-yl-imidazo[4,5-c]quinolin-1-yl)-phenyl]-propionitrile is obtained as described in Example 1 using 6-bromo-4,7-dichloro-3-nitro-quinoline (Example 84a), 3-(4-amino-3-chloro-phenyl)-propionitrile (Example 41a) and thiophene-2-boronic acid. 3-[3-Chloro-4-(7-chloro-8-thiophen-2-yl-imidazo[4,5-c]quinolin-1-yl)-phenyl]-propionitrile, analytical HPLC: t_{R}=7.98 minutes (Grad 1); ES⁺-MS: m/eₒ=449.2.

### Example 109

### 3-[3-Chloro-4-(7-chloro-8-thiophen-3-yl-imidazo[4,5-c]quinolin-1-yl)-phenyl]-propionitrile

3-[3-Chloro-4-(7-chloro-8-thiophen-3-yl-imidazo[4,5-c]quinolin-1-yl)-phenyl]-propionitrile is obtained as described in Example 1 using 6-bromo-4,7-dichloro-3-nitro-quinoline (Example 84a), 3-(4-amino-3-chloro-phenyl)-propionitrile (Example 41a) and thiophene-3-boronic acid. 3-[3-Chloro-4-(7-chloro-8-thiophen-3-yl-imidazo[4,5-c]quinolin-1-yl)-phenyl]-propionitrile, analytical HPLC: t_{R}=7.60 minutes (Grad 1); ES⁺-MS: m/eₒ=449.2.

### Example 110

### 3-{3-Chloro-4-[7-chloro-8-(1H-indol-5-yl)-imidazo[4,5-c]quinolin-1-yl]-phenyl}-propionitrile

3-{3-Chloro-4-[7-chloro-8-(1H-indol-5-yl)-imidazo[4,5-c]quinolin-1-yl]-phenyl}-propionitrile is obtained as described in Example 1 using 6-bromo-4,7-dichloro-3-nitro-quinoline (Example 84a), 3-(4-amino-3-chloro-phenyl)-propionitrile (Example 41a) and 5-indolylboronic acid. 3-{3-Chloro-4-[7-chloro-8-(1H-indol-5-yl)-imidazo[4,5-c]quinolin-1-yl]-phenyl}-propionitrile, analytical HPLC: t_{R}=7.29 minutes (Grad 1); ES⁺-MS: m/eₒ=481.8.

### Example 111

### 3-[3-Chloro-4-(7-chloro-8-thiophen-2-yl-imidazo[4,5-c]quinolin-1-yl)-phenyl]-propylamine

3-[3-Chloro-4-(7-chloro-8-thiophen-2-yl-imidazo[4,5-c]quinolin-1-yl)-phenyl]-propylamine is obtained as described in Example 4 using 3-[3-chloro-4-(7-chloro-8-thiophen-2-yl-imidazo[4,5-c]quinolin-1-yl)-phenyl]-propionitrile (Example 108) as starting material. 3-[3-Chloro-4-(7-chloro-8-thiophen-2-yl-imidazo[4,5-c]quinolin-1-yl)-phenyl]-propylamine, analytical HPLC: t_{R}=6.31 minutes (Grad 1); ES⁺-MS: m/eₒ=453.0.

### Example 112

### 3-[3-Chloro-4-(7-chloro-8-thiophen-3-yl-imidazo[4,5-c]quinolin-1-yl)-phenyl]-propylamine

3-[3-Chloro-4-(7-chloro-8-thiophen-3-yl-imidazo[4,5-c]quinolin-1-yl)-phenyl]-propylamine is obtained as described in Example 4 using 3-[3-chloro-4-(7-chloro-8-thiophen-3-yl-imidazo[4,5-c]quinolin-1-yl)-phenyl]-propionitrile (Example 109) as starting material. 3-[3-Chloro-4-(7-chloro-8-thiophen-3-yl-imidazo[4,5-c]quinolin-1-yl)-phenyl]-propylamine, analytical HPLC: t_{R}=6.22 minutes (Grad 1); ES⁺-MS: m/eₒ=453.0.

### Example 113

### 3-{3-Chloro-4-[7-chloro-8-(1H-indol-5-yl)-imidazo[4,5-c]quinolin-1-yl]-phenyl}-propylamine

3-{3-Chloro-4-[7-chloro-8-(1H-indol-5-yl)-imidazo[4,5-c]quinolin-1-yl]-phenyl}-propylamine is obtained as described in Example 4 using 3-{3-chloro-4-[7-chloro-8-(1H-indol-5-yl)-imidazo[4,5-c]quinolin-1-yl]-phenyl}-propionitrile (Example 110) as starting material. 3-{3-Chloro-4-[7-chloro-8-(1H-indol-5-yl)-imidazo[4,5-c]quinolin-1-yl]-phenyl}-propylamine, analytical HPLC: t_{R}=6.03 minutes (Grad 1); ES⁺-MS: m/eₒ=486.0.

### Example 114

### 3-[4-(2-Amino-7-chloro-8-thiophen-3-yl-imidazo[4,5-c]quinolin-1-yl)-phenyl]-propionitrile

3-[4-(2-Amino-7-chloro-8-thiophen-3-yl-imidazo[4,5-c]quinolin-1-yl)-phenyl]-propionitrile is obtained as described in Example 1 using 3-[4-(2-amino-8-bromo-7-chloro-imidazo[4,5-c]quinolin-1-yl)-phenyl]-propionitrile (Example 114a) and thiophene-3-boronic acid. 3-[4-(2-Amino-7-chloro-8-thiophen-3-yl-imidazo[4,5-c]quinolin-1-yl)-phenyl]-propionitrile, analytical HPLC: t_{R}=1.77 minutes (Grad 7); ES⁺-MS: m/e=430.1.

### Example 114a

### 3-[4-(2-Amino-8-bromo-7-chloro-imidazo[4,5-c]quinolin-1-yl)-phenyl]-propionitrile

1.8 g (4.48 mmol) of 3-[4-(3-amino-6-bromo-7-chloro-quinolin-4-ylamino)-phenyl]-propionitrile (Example 114b) are dissolved in 90 mL of ethanol and 1.4 g (0.75 mmol) of cyanogen bromide are added. The solution is stirred for 3 days at RT. After this time, the solvent is evaporated to dryness and the crude compound is purified by medium-pressure liquid chromatography to obtain 3-[4-(2-amino-8-bromo-7-chloro-imidazo[4,5-c]quinolin-1-yl)-phenyl]-propionitrile, analytical HPLC: t_{R}=1.68 minutes (Grad 7); ES⁺-MS: m/eₒ=428.0.

### Example 114b

### 3-[4-(3-Amino-6-bromo-7-chloro-quinolin-4-ylamino)-phenyl]-propionitrile

3-[4-(3-Amino-6-bromo-7-chloro-quinolin-4-ylamino)-phenyl]-propionitrile is obtained as described in Examples 1a to 1e using 6-bromo-4,7-dichloro-3-nitro-quinoline (Example 84a) and 3-(4-amino-phenyl)-propionitrile (Example 31a). 3-[4-(3-Amino-6-bromo-7-chloro-quinolin-4-ylamino)-phenyl]-propionitrile, analytical HPLC: t_{R}=2.11 minutes (Grad 7); ES⁺-MS: m/eₒ=433.0.

### Example 115

### 3-[4-(2-Amino-8-benzofuran-2-yl-7-chloro-imidazo[4,5-c]quinolin-1-yl)-phenyl]-propionitrile 3-[4-(2-Amino-8-benzofuran-2-yl-7-chloro-imidazo[4,5-c]quinolin-1-yl)-phenyl]-propionitrile is obtained as described in Example 1 using 3-[4-(2-amino-8-bromo-7-chloro-imidazo[4,5-c]quinolin-1-yl)-phenyl]-propionitrile (Example 114a) and benzo[b]furan-2-boronic acid. 3-[4-(2-Amino-8-benzofuran-2-yl-7-chloro-imidazo[4,5-c]quinolin-1-yl)-phenyl]-propionitrile, analytical HPLC: t_{R}=1.92 minutes (Grad 7); ES⁺-MS: m/eₒ=464.4.

### Example 116

### 1-[4-(3-Amino-propyl)-phenyl]-7-chloro-8-thiophen-3-yl-1H-imidazo[4,5-c]quinolin-2-ylamine

1-[4-(3-Amino-propyl)phenyl]-7-chloro-8-thiophen-3-yl-1H-imidazo[4,5-c]quinolin-2-ylamine is obtained as described in Example 4 using 3-[4-(2-amino-7-chloro-8-thiophen-3-yl-imidazo[4,5-c]quinolin-1-yl)-phenyl]-propionitrile (Example 114) as starting material. 1-[4-(3-Amino-propyl)-phenyl]-7-chloro-8-thiophen-3-yl-1H-imidazo[4,5-c]quinolin-2-ylamine, analytical HPLC: t_{R}=1.56 minutes (Grad 7); ES⁺-MS: m/eₒ=434.7.

### Example 117

### 1-[4-(3-Amino-propyl)-phenyl]-8-benzofuran-2-yl-7-chloro-1H-imidazo[4,5-c]quinolin-2-ylamine

1-[4-(3-Amino-propyl)-phenyl]-8-benzofuran-2-yl-7-chloro-1H-imidazo[4,5-c]quinolin-2-ylamine is obtained as described in Example 4 using 3-[4-(2-amino-8-benzofuran-2-yl-7-chloro-imidazo[4,5-c]quinolin-1-yl)-phenyl]-propionitrile (Example 115) as starting material. 1-[4-(3-Amino-propyl)-phenyl]-8-benzofuran-2-yl-7-chloro-1H-imidazo[4,5-c]quinolin-2-ylamine, analytical HPLC: t_{R}=1.70 minutes (Grad 7); ES⁺-MS: m/nₒ=468.7.

### Example 118

### 8-(2,4-Dimethoxy-phenyl)-1-(2-fluoro-phenyl)-1H-imidazo[4,5-c]quinoline

8-(2,4-Dimethoxy-phenyl)-1-(2-fluoro-phenyl)-1H-imidazo[4,5-c]quinoline is obtained as described in Example 1 using 2-fluoroaniline and 2,4-dimethoxyphenylboronic acid. 8-(2,4-Dimethoxy-phenyl)-1-(2-fluoro-phenyl)-1H-imidazo[4,5-c]quinoline, analytical HPLC: t_{R}=7.35 minutes (Grad 1); ES⁺-MS: m/eₒ=400.4.

### Example 119

### 8-(2,5-Dimethoxy-phenyl)-1-(2-fluoro-phenyl)-1H-imidazo[4,5-c]quinoline

8-(2,5-Dimethoxy-phenyl)-1-(2-fluoro-phenyl)-1H-imidazo[4,5-c]quinoline is obtained as described in Example 1 using 2-fluoroaniline and 2,5-dimethoxyphenylboronic acid. 8-(2,5-Dimethoxy-phenyl)-1-(2-fluoro-phenyl)-1H-imidazo[4,5-c]quinoline, analytical HPLC: t_{R}=7.38 minutes (Grad 1); ES⁺-MS: m/eₒ=400.2.

### Example 120

### 8-(3,4-Dimethoxy-phenyl)-1-(2-fluoro-phenyl)-1H-imidazo[4,5-c]quinoline

8-(3,4-Dimethoxy-phenyl)-1-(2-fluoro-phenyl)-1H-imidazo[4,5-c]quinoline is obtained as described in Example 1 using 2-fluoroaniline and 3,4-dimethoxyphenylboronic acid. 8-(3,4-Dimethoxy-phenyl)-1-(2-fluoro-phenyl)-1H-imidazo[4,5-c]quinoline, analytical HPLC: t_{R-}=7.09 minutes (Grad 1); ES⁺-MS: m/eₒ=400.3.

### Example 121

### 1-(2-Fluoro-phenyl)-8-phenyl-1H-imidazo[4,5-c]quinoline

1-(2-Fluoro-phenyl)-8-phenyl-1H-imidazo[4,5-c]quinoline is obtained as described in Example 1 using 2-fluoroaniline and phenylboronic acid. 1-(2-Fluoro-phenyl)-8-phenyl-1H-imidazo[4,5-c]quinoline, analytical HPLC: t_{R}=9.48 minutes (Grad 6); ES⁺-MS: m/eₒ=340.3.

### Example 122

### 1-(2-Fluoro-phenyl)-8-(3,4,5-trimethoxy-phenyl)-1H-imidazo[4,5-c]quinoline

1-(2-Fluoro-phenyl)-8-(3,4,5-trimethoxy-phenyl)-1H-imidazo[4,5-c]quinoline is obtained as described in Example 1 using 2-fluoroaniline and 3,4,5-trimethoxyphenylboronic acid. 1-(2-Fluoro-phenyl)-8-(3,4,5-trimethoxy-phenyl)-1H-imidazo[4,5-c]quinoline, analytical HPLC: t_{R}=7.14 minutes (Grad 1); ES⁺-MS: m/eₒ=430.4.

### Example 123

### 8-(2,3-Dimethoxy-phenyl)-1-(2-fluoro-phenyl)-1H-imidazo[4,5-c]quinoline

8-(2,3-Dimethoxy-phenyl)-1-(2-fluoro-phenyl)-1H-imidazo[4,5-c]quinoline is obtained as described in Example 1 using 2-fluoroaniline and 2,3-dimethoxyphenylboronic acid. 8-(2,3-Dimethoxy-phenyl)-1-(2-fluoro-phenyl)-1H-imidazo[4,5-c]quinoline, analytical HPLC: t_{R}=6.87 minutes (Grad 1); ES⁺-MS: m/eₒ=400.2.

### Example 124

### 1-(2-Fluoro-phenyl)-8-(2,3,4-trimethoxy-phenyl)-1H-imidazo[4,5-c]quinoline

1-(2-Fluoro-phenyl)-8-(2,3,4-trimethoxy-phenyl)-1H-imidazo[4,5-c]quinoline is obtained as described in Example 1 using 2-fluoroaniline and 2,3,4-trimethoxyphenylboronic acid. 1-(2-Fluorophenyl)-8-(2,3,4-trimethoxy-phenyl)-1H-imidazo[4,5-c]quinoline, analytical HPLC: t_{R}=7.24 minutes (Grad 1); ES⁺-MS: m/eₒ=430.4.

### Example 125

### 1-(2-Fluoro-phenyl)-8-pyridin-4-yl-1H-imidazo[4,5-c]quinoline

1-(2-Fluoro-phenyl)-8-pyridin-4-yl-1H-imidazo[4,5-c]quinoline is obtained as described in Example 1 using 2-fluoroaniline and pyridine-4-boronic acid. 1-(2-Fluoro-phenyl)-8-pyridin-4-yl-1H-imidazo[4,5-c]quinoline, analytical HPLC: t_{R}=5.39 minutes (Grad 1); ES⁺-MS: m/eₒ=341.0.

### Example 126

### 1-(2-Fluoro-phenyl)-8-pyridin-3-yl-1H-imidazo[4,5-c]quinoline

1-(2-Fluoro-phenyl)-8-pyridin-3-yl-1H-imidazo[4,5-c]quinoline is obtained as described in Example 1 using 2-fluoroaniline and pyridine-3-boronic acid. 1-(2-Fluoro-phenyl)-8-pyridin-3-yl-1H-imidazo[4,5-c]quinoline, analytical HPLC: t_{R}=5.87 minutes (Grad 1); ES⁺-MS: m/eₒ=341.0.

### Example 127

### 4-[1-(2-Fluoro-phenyl)-1H-imidazo[4,5-c]quinolin-8-yl]-phenol

4-[1-(2-Fluoro-phenyl)-1H-imidazo[4,5-c]quinolin-8-yl]-phenol is obtained as described in Example 1 using 2-fluoroaniline and 4-hydroxyphenylboronic acid. 4-[1-(2-Fluoro-phenyl)-1H-imidazo[4,5-c]quinolin-8-yl]-phenol, analytical HPLC: t_{R}=6.67 minutes (Grad 1); ES⁺-MS: m/eₒ=356.4.

### Example 128

### 1-(2-Fluoro-phenyl)-8-(3-methoxy-phenyl)-1H-imidazo[4,5-c]quinoline

1-(2-Fluoro-phenyl)-8-(3-methoxy-phenyl)-1H-imidazo[4,5-c]quinoline is obtained as described in Example 1 using 2-fluoroaniline and 3-methoxyphenylboronic acid. 1-(2-Fluoro-phenyl)-8-(3-methoxy-phenyl)-1H-imidazo[4,5-c]quinoline, analytical HPLC: t_{R}=7.55 minutes (Grad 1); ES⁺-MS: m/eₒ=370.2.

### Example 129

### {3-[1-(2-Fluoro-phenyl)-1H-imidazo[4,5-c]quinolin-8-yl]-benzyl}-dimethyl-amine 19 mg (0.23 mmol) of sodium acetate and 22.5 μL (0.13 mmol) of dimethylamine are added to a solution of 42 mg (0.11 mmol) of 3-[1-(2-fluoro-phenyl)-1H-imidazo[4,5-c]quinolin-8-yl]-benzaldehyde (Example 129a) in 5 mL of MeOH/AcOH (1:1). The solution is stirred for 10 min at RT and then 62 mg (0.13 mmol) of PS—BH₃CN are added. After stirring for 18 hours at RT, the suspension is filtered and evaporated to dryness. The crude product is purified by medium-pressure liquid chromatography to provide {3-[1-(2-Fluoro-phenyl)-1H-imidazo[4,5-c]quinolin-8-yl]-benzyl}-dimethyl-amine, analytical HPLC: t_{R}=6.51 minutes (Grad 1); ES⁺-MS: m/eₒ=397.4.

### Example 129a

### 3-[1-(2-Fluoro-phenyl)-1H-imidazo[4,5-c]quinolin-8-yl]-benzaldehyde

3-[1-(2-Fluorophenyl)-1H-imidazo[4,5-c]quinolin-8-yl]-benzaldehyde is obtained as described in Example 1 using 2-fluoroaniline and 3-formylphenylboronic acid. 3-[1-(2-Fluoro-phenyl)-1H-imidazo[4,5-c]quinolin-8-yl]-benzaldehyde, analytical HPLC: t_{R}=7.48 minutes (Grad 1); ES⁺-MS: m/eₒ=368.1.

### Example 130

### 1-(2-Fluoro-phenyl)-8-[3-(4-methyl-piperazin-1-ylmethyl)-phenyl]-1H-imidazo[4,5-c]quinoline

1-(2-Fluoro-phenyl)-8-[3-(4-methyl-piperazin-1-ylmethyl)-phenyl]-1H-imidazo[4,5-c]quinoline is obtained as described in Example 129 using 1-methylpiperazine. 1-(2-Fluoro-phenyl)-8-[3-(4-methyl-piperazin-1-ylmethyl)-phenyl]-1H-imidazo[4,5-c]quinoline, analytical HPLC: t_{R}=6.35 minutes (Grad 1); ES⁺-MS: m/eₒ=452.0.

### Example 131

### 1-(2-Fluoro-phenyl)-8-(3-morpholin-4-ylmethyl-phenyl)-1H-imidazo[4,5-c]quinoline

1-(2-Fluoro-phenyl)-8-(3-morpholin-4-ylmethyl-phenyl)-1H-imidazo[4,5-c]quinoline is obtained as described in Example 129 using morpholine. 1-(2-Fluoro-phenyl)-8-(3-morpholin-4-ylmethyl-phenyl-1H-imidazo[4,5c]quinoline, analytical HPLC: t_{R}=6.60 minutes (Grad 1); ES⁺-MS: m/eₒ=439.0.

### Example 132

### 1-(2-Fluoro-phenyl)-8-(3-piperazin-1-ylmethyl-phenyl)-1H-imidazo[4,5-c]quinoline

1-(2-Fluoro-phenyl)-8-(3-piperazin-1-ylmethyl-phenyl)-1H-imidazo[4,5-c]quinoline is obtained as described in Example 129 using piperazine. 1-(2-Fluoro-phenyl)-8-(3-piperazin-1-ylmethyl-phenyl)-1H-imidazo[4,5-c]quinoline, analytical HPLC: t_{R}=6.27 minutes (Grad 1); ES⁺-MS: m/eₒ=438.5.

### Example 133

### 1-(2-Fluoro-phenyl)-8-(3-pyrrolidin-1-ylmethyl-phenyl)-1H-imidazo[4,5-c]quinoline

1-(2-Fluoro-phenyl)-8-(3-pyrrolidin-1-ylmethyl-phenyl)-1H-imidazo[4,5-c]quinoline is obtained as described in Example 129 using pyrrolidine. 1-(2-Fluoro-phenyl)-8-(3-pyrrolidin-1-ylmethyl-phenyl)-1H-imidazo[4,5-c]quinoline, analytical HPLC: t_{R}=6.68 minutes (Grad 1); ES⁺-MS: m/eₒ=423.0.

### Example 134

### 1-Phenyl-8-(3-piperazin-1-yl-phenyl)-1H-imidazo[4,5-c]quinoline

75 mg (0.22 mmol) of 8-(3-fluoro-phenyl)-1-phenyl-1H-imidazo[4,5-c]quinoline (Example 134a) and 0.38 mL of piperazine are added to 1.7 mL of NMP. The solution is heated for 5 hours at 310° C. using a Woodsches-metal bath. After this time, the crude compound is purified by medium-pressure liquid chromatography to provide 1-phenyl-8-(3-piperazin-1-yl-phenyl)-1H-imidazo[4,5-c]quinoline, analytical HPLC: t_{R}=1.53 minutes (Grad 7); ES⁺-MS: m/eₒ=406.5.

### Example 134a

### 8-(3-Fluoro-phenyl)-1-phenyl-1H-imidazo[4,5-c]quinoline

8-(3-Fluoro-phenyl)-1-phenyl-1H-imidazo[4,5-c]quinoline is obtained as described in Example 1 using aniline and 3-fluorophenylboronic acid. 8-(3-Fluoro-phenyl)-1-phenyl-1H-imidazo[4,5-c]quinoline, analytical HPLC: t_{R}=1.63 minutes (Grad 7); ES⁺-MS: m/eₒ=340.1.

### Example 135

### 8-[3-(4-Methyl-piperazin-1-yl)-phenyl]-1-phenyl-1H-imidazo[4,5-c]quinoline

8-[3-(4-Methyl-piperazin-1-yl)-phenyl]-1-phenyl-1H-imidazo[4,5-c]quinoline is obtained as described in Example 134 using N-methylpiperazine. 8-[3-(4-Methyl-piperazin-1-yl)-phenyl]-1-phenyl-1H-imidazo[4,5-c]quinoline, analytical HPLC: t_{R}=1.52 minutes (Grad 7); ES⁺-MS: m/eₒ=420.5.

### Example 136

### 4-{1-[4-(4-Methyl-piperazin-1-yl)-phenyl]-1H-imidazo[4,5-c]quinolin-8-yl}-phenol

4-{1-[4-(4-Methyl-piperazin-1-yl)-phenyl]-1H-imidazo[4,5-c]quinolin-8-yl}-phenol is obtained as described in Example 1 using 8-bromo-1-[4-(4-methyl-piperazin-1-yl)-phenyl]-1H-imidazo[4,5-c]quinoline (Example 136a) and 4-hydroxyphenylboronic acid.

### Example 136a

### 8-Bromo-1-[4-(4-methyl-piperazin-1-yl)-phenyl]-1H-imidazo[4,5-c]quinoline

1.2 g (3.5 mmol) of 8-bromo-1-(4-fluoro-phenyl)-1H-imidazo[4,5-c]quinoline (Example 136b) are dissolved in 20 mL of DMSO, and 1.45 g (10.5 mmol) of potassium carbonate and 7.7 mL (69.7 mmol) of N-methylpyperazine are added. The suspension is stirred for 2 days at 120° C. and then added to 100 mL of ethyl acetate. After washing with water and brine, the organic solution is evaporated to dryness and the crude compound is purified by medium-pressure liquid chromatography to provide 8-Bromo-1-[4-(4-methyl-piperazin-1-yl)phenyl]-1H-imidazo[4,5-c]quinoline.

### Example 136b

### 8-bromo-1-(4-fluoro-phenyl)-1H-imidazo[4,5-c]quinoline

### 8-bromo-1-(4-fluoro-phenyl)-1H-imidazo[4,5-c]quinoline is obtained as described in Example 1 using 4-fluoroaniline. 8-bromo-1-(4-fluoro-phenyl)-1H-imidazo[4,5-c]quinoline, analytical HPLC: t_{R}=7.21 minutes (Grad 1); ES⁺-MS: m/eₒ=342.1, 344.1.

### Example 137

### 1-[4-(4-Methyl-piperazin-1-yl)-phenyl]-8-phenyl-1H-imidazo[4,5-c]quinoline

1-[4-(4-Methyl-piperazin-1-yl)-phenyl]-8-phenyl-1H-imidazo[4,5-c]quinoline is obtained as described in Example 136 using phenylboronic acid. 1-[4-(4-Methyl-piperazin-1-yl)-phenyl]-8-phenyl-1H-imidazo[4,5-c]quinoline, analytical HPLC: t_{R}=6.10 minutes (Grad 1); ES⁺-MS: m/eₒ=420.4.

### Example 138

### [4-(8-Pyridin-4-yl-imidazo[4,5-c]quinolin-1-yl)-phenyl]-acetonitrile

[4-(8-Pyridin-4-yl-imidazo[4,5-c]quinolin-1-yl)-phenyl]-acetonitrile is obtained as described in Example 1 using (4-amino-phenyl)-acetonitrile and pyridine-4-boronic acid. [4-(8-Pyridin-4-yl-imidazo[4,5-c]quinolin-1-yl)-phenyl]-acetonitrile, analytical HPLC: t_{R}=6.34 minutes (Grad 1); ES⁺-MS: m/eₒ=363.3.

### Example 139

### 1,8-Diphenyl-1H-imidazo[4,5-c]quinoline

1,8-Diphenyl-1H-imidazo[4,5-c]quinoline is obtained as described in Example 1 using aniline and phenylboronic acid. 1,8-Diphenyl-1H-imidazo[4,5c]quinoline, analytical HPLC: t_{R}=9.64 minutes (Grad 1); ES⁺-MS: m/eₒ=322.3.

In the following Examples 140-143 providing activity determinations of compounds of the preceding examples, the following letters are intended to symbolize the following IC₅₀ values (only examples with concrete measurement results are represented):

| Letter | IC₅₀ Range Class |
|---|---|
| A | ≦0.5 μM |
| B | >0.5 μM up to 1 μM |
| C | >1 μM up to 2 μM |

### Example 140

### Inhibition of RET by Compounds of the Present Invention

Using the testing method described above, with the following test compounds of formula (I), the following IC₅₀ values for inhibition of RET are obtained:

| Compound of Example | IC₅₀ Range Class |
|---|---|
| 119 | B |
| 125 | B/A |
| 127 | A |
| 129 | A |
| 134 | B |

### Example 141

### Inhibition of ALK by Compounds of the Present Invention

Using the test system described above, with the following test compounds of formula (I), the following IC₅₀ values for inhibition of ALK are obtained:

| Compound of Example | IC₅₀ Range Class |
|---|---|
| 18 | B/A |
| 11 | A |
| 48 | A |

### Example 142

### Inhibition of PKB by Compounds of the Present Invention

Using the test system described above, with the following test compounds of formula (I), the following IC₅₀ values for inhibition of PKB are obtained:

| Compound of Example | IC₅₀ Range Class |
|---|---|
| 61 | A |
| 64 | A |
| 74 | A |
| 90 | A |

### Example 143

### Inhibition of S6K1 by Compounds of the Present Invention

Using the testing method described above, with the following test compounds of formula (I), the following IC₅₀ values for inhibition of S6K1 are obtained:

| Compound of Example | IC₅₀ Range Class |
|---|---|
| 11 | A |
| 18 | A |
| 22 | A |
| 30 | A |
| 83 | A |

### Example 144

### Tablets 1 Comprising Compounds of the Formula (I)

Tablets comprising, as active ingredient, 50 mg of any one of the compounds of formula (I) mentioned in the preceding Examples 1-139 of the following composition are prepared using routine methods:

Composition

| | | |
|---|---|---|
| Active Ingredient | 50 | mg |
| Wheat starch | 60 | mg |
| Lactose | 50 | mg |
| Colloidal silica | 5 | mg |
| Talcum | 9 | mg |
| Magnesium stearate | 1 | mg |
| | 175 | mg |

Manufacture: The active ingredient is combined with part of the wheat starch, the lactose and the colloidal silica and the mixture pressed through a sieve. A further part of the wheat starch is mixed with the 5-fold amount of water on a water bath to form a paste and the mixture made first is kneaded with this paste until a weakly plastic mass is formed.

The dry granules are pressed through a sieve having a mesh size of 3 mm, mixed with a pre-sieved mixture (1 mm sieve) of the remaining corn starch, magnesium stearate and talcum and compressed to form slightly biconvex tablets.

### Example 145

### Tablets 2 Comprising Compounds of the Formula (I)

Tablets, comprising, as active ingredient, 100 mg of any one of the compounds of formula (I) of Examples 1-139 are prepared with the following composition, following standard procedures:

Composition

| | | |
|---|---|---|
| Active Ingredient | 100 | mg |
| Crystalline lactose | 240 | mg |
| Avicel | 80 | mg |
| PVPPXL | 20 | mg |
| Aerosil | 2 | mg |
| Magnesium stearate | 5 | mg |
| | 447 | mg |

Manufacture: The active ingredient is mixed with the carrier materials and compressed by means of a tabletting machine (Korsch EKO, Stempeldurchmesser 10 mm).

### Example 146

### Capsules

Capsules, comprising, as active ingredient, 100 mg of any one of the compounds of formula (I), given in Examples 1-139, of the following composition are prepared according to standard procedures:

Composition

| | | |
|---|---|---|
| Active Ingredient | 100 | mg |
| Avicel | 200 | mg |
| PVPPXL | 15 | mg |
| Aerosil | 2 | mg |
| Magnesium stearate | 1.5 | mg |
| | 318.5 | mg |

Manufacturing is done by mixing the components and filling them into hard gelatine capsules, size 1.

1. A compound of formula (I): 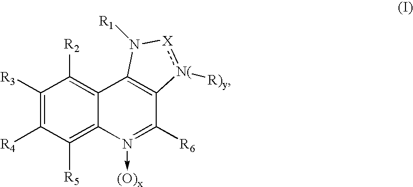
wherein
each of x and y is, independently of the other, 0 or 1;
R₁ is an organic moiety that can be bound to nitrogen;
X is C═O (especially preferred) or C═S with the proviso that then the dashed line bonding X to N is absent, so that X is bound to the adjacent N via a single bond and with the proviso that then y is 1 and R is hydrogen or an organic moiety that can be bound to nitrogen, or
X is (CR₇), wherein R₇ is hydrogen or an organic or inorganic moiety with the proviso that then the dashed line bonding X to N is a bond, so that X is bound to the adjacent N via a double bond, and with the proviso that then y is zero or y is 1 and then —R is →O; and
each of R₂, R₃, R₄, R₅ and R₆, independently of the others, is an organic moiety or hydrogen or an inorganic moiety, with the proviso that R₃ cannot be unsubstituted phenyl unless R₁ is phenyl substituted with a heterocyclic ring;
or a pharmaceutically acceptable salt thereof.

2. A compound according to claim 1,
wherein
each of x and y is, independently of the other, 0 or 1;
R₁ is substituted or unsubstituted aryl or heteroaryl, especially phenyl, which is substituted with up to 4 substituents, preferably up to 3 substituents, where the substituents are the same or different and are independently selected from halo, e.g., F or Cl; C₁-C₇lower alkyl which may be unsubstituted or substituted with halo, especially methyl, ethyl, propyl or trifluoromethyl; cyano; cyano-lower alkyl, e.g., cyanomethyl, cyanoethyl or cyanopropyl; lower alkoxy; amino; amino-lower alkyl; amino-lower alkoxy; amino-lower alkyl sulfanyl; or thiol-lower alkyl, wherein the amino group can be mono- or di-substituted, e.g., —(C₁-C₇)ₘNR₈R₉ or —O—(C₁-C₇)ₘNR₈R₉,
wherein
m is 0 or 1; and
R₈ and R₉ can be the same or different and are independently H; lower alkyl, e.g., methyl, ethyl or propyl; lower cycloalkyl, e.g., cyclopropyl, or
R₈ and R₉, together with the N atom, form a 3- to 8-membered heterocyclic ring containing 1-4 nitrogen, oxygen or sulfur atoms, e.g., azetidinyl, pyrrolidinyl, piperidino, morpholinyl, imidazolinyl, imidazolinyl-ethyl, piperazinyl or lower alkyl-piperazinyl; amino-carbonyl-lower alkyl, e.g., R₈R₉—N—C(O)—CH₂—, wherein R₈ and R₉ are as defined above; heterocyclyl; heterocyclyl-lower alkyl; heterocyclyl-lower alkoxy; or heterocyclyl-lower alkanesulfanyl, wherein the heterocyclyl is a 3- to 8-membered heterocyclic ring containing 1-4 nitrogen, oxygen or sulfur atoms, e.g., imidazolyl, imidazolinyl, pyrrolidinyl, morpholinyl, azetidinyl, pyridyl, piperidino, piperidyl, piperazinyl or lower alkyl-piperazinyl; substituted or unsubstituted amide; amide-lower alkyl, e.g., —CH₂—CH(NH₂)—C(O)—NH₂), wherein alkyl may be linear or cyclic, e.g., cyclopropylene; and the alkyl in any of the substituents above may optionally be substituted with —NR₈R₉, wherein R₈ and R₉ are as defined above;
X is C═O or C═S, with the proviso that then the dashed line bonding X to N is absent, so that X is bound to the adjacent N via a single bond and with the proviso that then y is 1 and R is hydrogen or an organic moiety that can be bound to nitrogen, or
X is (CR₇), wherein R₇ is hydrogen or an organic moiety, such as C₁-C₇lower alkyl; amino; or amino alkyl, wherein the alkyl may be unsubstituted or substituted with halo, e.g., methyl, ethyl, propyl or trifluoromethyl; lower alkoxy, e.g., methoxy; or cycloalkyl, e.g., cyclopropyl; with the proviso that then the dashed line bonding X to N is a bond, so that X is bound to the adjacent N via a double bond; and with the proviso that then y is zero, or y is 1 and then —R is →O;
R₂ is hydrogen;
R₃ is unsubstituted or substituted C₅-C₁₄heterocyclyl, e.g., thienyl, benzo[1,3]dioxolo, indolyl, benzofuranyl or pyridiyl; unsubstituted or substituted C₅-C₁₄aryl, e.g., phenyl or phenyl substituted with up to 4 substituents, preferably up to 3 substituents, which are the same or different and are selected from halo, e.g., Cl or F; hydroxy; C₁-C₄lower alkoxy, e.g., methoxy; lower alkyl, e.g., methyl; or —(C₁-C₄)ₘNR₈R₉,
wherein
m is 0 or 1; and
R₈ and R₉, are as defined above, e.g., piperazinyl, methylpiperazinyl, morpholinyl or pyrrolidinyl;
R₄ is hydrogen or halo, e.g., fluoro or chloro;
R₅ is hydrogen; and
R₆ is hydrogen, amino, amino alkyl or alkylamido, e.g., methylamido —NHC(O)—CH₃), with the proviso that R₃ cannot be unsubstituted phenyl unless R₁ is phenyl substituted with an heterocyclic ring;
or a pharmaceutically acceptable salt thereof.

3. A compound according to claim 1,
wherein
each of x and y is, independently of the other, 0 or 1;
R₁ is substituted or unsubstituted phenyl where the phenyl is substituted with up to 4 substituents, preferably up to 3 substituents, where the substituents are the same or different and are independently selected from halo, e.g., F or Cl; C₁-C₇lower alkyl, which may be unsubstituted or substituted with halo, especially methyl, ethyl, propyl or trifluoromethyl; cyano; cyano-lower alkyl, e.g., cyanomethyl, cyanoethyl or cyanopropyl; amino; amino-lower alkyl, wherein the amino group can be mono- or di-substituted, e.g., —(C₁-C₇)ₘNR₈R₉ or —O—(C₁-C₇)ₘNR₈R₉,
wherein
m is 0 or 1; and
R₈ and R₉ can be the same or different and are independently H; lower alkyl, e.g., methyl, ethyl or propyl; lower cycloalkyl, e.g., cyclopropyl, or
R₈ and R₉, together with the N atom, form a 3- to 8-membered heterocyclic ring containing 1-4 nitrogen, oxygen or sulfur atoms, e.g., imidazolinyl, imidazolinyl-ethyl, piperazinyl or lower alkyl-piperazinyl; amino-carbonyl-lower alkyl, e.g., R₈R₉—N—C(O)—CH₂—, wherein R₈ and R₉ are as defined above; heterocyclyl; heterocyclyl-lower alkyl; heterocyclyl-lower alkoxy; or heterocyclyl-lower alkanesulfanyl, wherein the heterocyclyl is a 3- to 8-membered heterocyclic ring containing 1-4 nitrogen, oxygen or sulfur atoms, e.g., imidazolinyl, imidazolinyl, imidazolinyl-ethyl, piperazinyl or lower alkyl-piperazinyl; substituted or unsubstituted amide; amide-lower alkyl, e.g., —CH₂—CH(NH₂)—C(O)—NH₂, wherein alkyl may be linear or cyclic, e.g., cyclopropylene; and the alkyl in any of the substituents above may optionally be substituted with —NR₈R₉, wherein R₈ and R₉ are as defined above;
X is (CR₇), wherein R₇ is hydrogen; lower alkyl, e.g., methyl or ethyl; amino; or amino alkyl, with the proviso that then the dashed line bonding X to N is a bond, so that X is bound to the adjacent N via a double bond, and with the proviso that then y is zero, or y is 1 and then —R is →O;
R₂ is hydrogen;
R₃ is unsubstituted or substituted C₅-C₁₄heterocyclyl, e.g., thienyl, benzo[1,3]dioxolo, indolyl, benzofuranyl or pyridiyl; unsubstituted or substituted C₅-C₁₄aryl, e.g., phenyl or phenyl substituted with up to 4 substituents, preferably up to 3 substituents, which are the same or different and are selected from halo, e.g., Cl or F; hydroxy; C₁-C₄lower alkoxy, e.g., methoxy; lower alkyl, e.g., methyl; or —(C₁-C₄)ₘNR₈R₉,
wherein
m is 0 or 1; and
R₈ and R₉ are as defined above, e.g., piperazinyl, methylpiperazinyl, morpholinyl or pyrrolidinyl;
R₄ is hydrogen or halo, (e.g. fluoro or chloro);
R₅ is hydrogen; and
R₆ is hydrogen, with the proviso that R₃ cannot be unsubstituted phenyl unless R₁ is phenyl substituted with an heterocyclic ring;
or a pharmaceutically acceptable salt thereof.

4. A pharmaceutical composition comprising a compound of formula (I), according to claim 1, or a pharmaceutically acceptable salt thereor, and a pharmaceutically acceptable carrier.

5. A compound of the formula (I), according to claim 1, for use in the treatment of the animal or human body, especially in the treatment of a protein kinase dependent disease.

6. A compound according to claim 5, wherein the protein kinase dependent disease is preferably one depending on PKB, ALK, S6K1 or RET and (especially aberrantly highly-expressed or activated) PKB, ALK, S6K1 or RET-dependent disease or disease dependent on the activation of the PKB, ALK, S6K1 or RET pathways, or a disease dependent on any two or more of the kinases just mentioned.

7-9. (canceled)

10. A method of treatment of a disease that responds to inhibition of a protein kinase, which comprises administering a prophylactically or therapeutically effective amount of a compound of formula (I) according to claim 1, or a pharmaceutically acceptable salt thereof, to a warm-blooded animal, e.g., a human, in need of such treatment.

11. A method according to claim 10, wherein the disease to be treated is a proliferative disease, preferably a benign or especially malignant tumor, more preferably carcinoma of the brain, kidney, liver, adrenal gland, bladder, breast, stomach, gastric tumors, ovaries, colon, rectum, prostate, pancreas, lung, vagina, thyroid, sarcoma, glioblastomas, multiple myeloma, gastrointestinal cancer, colon carcinoma, colorectal adenoma, tumor of the neck and head, an epidermal hyperproliferation, psoriasis, prostate hyperplasia, a neoplasia, especially of epithelial character, mammary carcinoma, leukemia, metabolic diseases, type II diabetes, obesity, hyperlipidemia and atherosclerosis.

12. A compound according to formula (I), for use in the treatment of a protein kinase dependent disease, especially one depending on ALK, S6K1 or RET and (especially aberrantly highly-expressed or activated) ALK, S6K1 or RET-dependent disease or disease dependent on the activation of the ALK, S6K1 or RET pathways or disease comprising administering a compound according to formula (I) 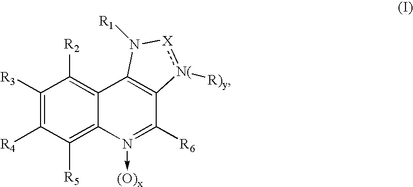
wherein
each of x and y is, independently of the other, 0 or 1;
R₁ is an organic moiety that can be bound to nitrogen;
X is C═O, especially preferred or C═S, with the proviso that then the dashed line bonding X to N is absent, so that X is bound to the adjacent N via a single bond and with the proviso that then y is 1 and R is hydrogen or an organic moiety that can be bound to nitrogen, or
X is (CR₇), wherein R₇ is hydrogen or an organic or inorganic moiety with the proviso that then the dashed line bonding X to N is a bond, so that X is bound to the adjacent N via a double bond, and with the proviso that then y is zero or y is 1 and then —R is →O; and
each of R₂, R₃, R₄, R₅ and R₆, independently of the others, is an organic moiety or hydrogen or an inorganic moiety;
or a pharmaceutically acceptable salt thereof.

13. A compound according to claim 12, comprising a the compound of the formula (I), or a pharmaceutically acceptable salt thereof,
wherein
each of x and y is, independently of the other, 0 or 1;
R₁ is phenyl or phenyl-lower alkyl, each of which, in the phenyl moiety, is unsubstituted or substituted by up to three moieties independently selected from halogen, especially fluoro, chloro, bromo or iodo; lower alkyl, especially methyl or ethyl; halo-lower alkyl, especially trifluoromethyl; hydroxy; lower alkoxy, especially methoxy; C₆-C₁₄aryl, especially phenyl; hydroxy-lower alkyl, especially 2-hydroxyethyl or hydroxymethyl; amino; amino-lower alkyl, especially aminomethyl or 2-aminoethyl; amidino; N-hydroxy-amidino; amidino-lower alkyl, such as 2-amidinoethyl; N-hydroxyamidino-lower alkyl, especially N-hydroxy-amidino-methyl or -2-ethyl; cyano-lower alkyl, especially cyanomethyl; and cyano or is C₃-C₈cycloalkyl, especially cyclohexyl; or hydroxyC₃-C₈cycloalkyl, especially hydroxy-cyclohexyl;
X is C═O or C═S, with the proviso that then the dashed line bonding X to N is absent, so that X is bound to the adjacent N via a single bond, and with the proviso that then y is 1 and R is hydrogen; lower alkyl, especially methyl, ethyl, n-propyl, isopropyl, n-butyl, isobutyl, 2,2-dimethylpropyl or 2-ethyl-n-butyl; mono- or di-hydroxy-lower alkyl, especially 2,3-dihydroxy-propyl or 3-hydroxy-2,2-dimethylpropyl; C₆-C₁₄aryl, which is unsubstituted or substituted by 1-3 substituents selected from lower alkyl, especially methyl or ethyl; halo-lower alkyl, especially trifluoromethyl; halogen, especially chloro; amino; lower alkanoylamino; lower alkoxy, especially methoxy and nitro; C₃-C₈cycloalkyl, especially cyclopropylmethyl or cyclohexylmethyl; or furanyl-lower alkyl, especially 3-furanyl-methyl, or
X is (CR₇), wherein R₇ is hydrogen or an organic or inorganic moiety that can be bound to nitrogen with the proviso that then the dashed line bonding X to N is a bond, so that X is bound to the adjacent N via a double bond, and with the proviso that then y is zero, or y is 1 and then —R is →O;
R₂ is hydrogen;
R₃ is hydrogen; lower alkyl, especially ethyl; halo, especially fluoro, chloro or bromo;
lower alkoxy, especially methoxy; or unsubstituted or substituted C₆-C₁₄aryl, especially phenyl, hydroxyphenyl or methoxyphenyl;
R₄ is hydrogen or halo, especially chloro;
R₅ is hydrogen or lower alkoxy, especially n-lower hexyloxy; and
R₆ is hydrogen, halo, especially chloro; C₆-C₁₄aryl, especially phenyl; C₃-C₈cycloalkyl, especially cyclopropyl; amino; lower alkyl-amino, especially methylamino or n-butylamino; hydroxy-lower alkylamino, especially 2-hydroxyethyl-amino; or
C₆-C₁₄arylcarbonylamino, especially benzoylamino.

14. A method according to claim 16, for treatment of a protein kinase dependent disease, especially one depending on ALK, S6K1 or RET and (especially aberrantly highly-expressed or activated) ALK, S6K1 or RET-dependent disease or disease dependent on the activation of the ALK, S6K1 or RET pathways or disease.

15. A compound according to claim 1, selected from the group consisting of:
[4-(8-Benzo[1,3]dioxol-5-yl-imidazo[4,5-c]quinolin-1-yl)-phenyl]-acetonitrile;
[4-(8-Thiophen-2-yl-imidazo[4,5-c]quinolin-1-yl)-phenyl]-acetonitrile;
[4-(8-Benzofuran-2-yl-imidazo[4,5-c]quinolin-1-yl)-phenyl]-acetonitrile;
2-[4-(8-Benzo[1,3]dioxol-5-yl-imidazo[4,5-c]quinolin-1-yl)-phenyl]-ethylamine;
2-[4-(8-Thiophen-2-yl-imidazo[4,5-c]quinolin-1-yl)-phenyl]-ethylamine;
2-[4-(8-Benzofuran-2-yl-imidazo[4,5-c]quinolin-1-yl)-phenyl]-ethylamine;
[3-Chloro-4-(8-thiophen-2-yl-imidazo[4,5-c]quinolin-1-yl)-phenyl]-acetonitrile;
(4-Amino-3-chloro-phenyl)-acetonitrile;
{3-Chloro-4-[8-(1H-indol-5-yl)-imidazo[4,5-c]quinolin-1-yl]-phenyl}-acetonitrile;
[3-Chloro-4-(8-thiophen-3-yl-imidazo[4,5-c]quinolin-1-yl)-phenyl]-acetonitrile;
2-[3-Chloro-4-(8-thiophen-2-yl-imidazo[4,5-c]quinolin-1-yl)-phenyl]-ethylamine;
2-{3-Chloro-4-[8-(1H-indol-5-yl)-imidazo[4,5-c]quinolin-1-yl]-phenyl}-ethylamine;
2-[3-Chloro-4-(8-thiophen-3-yl-imidazo[4,5-c]quinolin-1-yl)-phenyl]-ethylamine;
[2-Fluoro-4-(8-thiophen-2-yl-imidazo[4,5-c]quinolin-1-yl)-phenyl]-acetonitrile;
[4-(8-Benzofuran-2-yl-imidazo[4,5-c]quinolin-1-yl)-2-fluoro-phenyl]-acetonitrile;
{2-Fluoro-4-[8-(1H-indol-5-yl)-imidazo[4,5-c]quinolin-1-yl]-phenyl}-acetonitrile;
2-[2-Fluoro-4-(8-thiophen-2-yl-imidazo[4,5-c]quinolin-1-yl)-phenyl]-ethylamine;
2-[4-(8-Benzofuran-2-yl-imidazo[4,5-c]quinolin-1-yl)-2-fluoro-phenyl]-ethylamine;
2-{2-Fluoro-4-[8-(1H-indol-5-yl)-imidazo[4,5-c]quinolin-1-yl]-phenyl}-ethylamine;
[3-Methyl-4-(8-thiophen-3-yl-imidazo[4,5-c]quinolin-1-yl)-phenyl]-acetonitrile;
{4-[8-(1H-Indol-5-yl)-imidazo[4,5-c]quinolin-1-yl]-3-methyl-phenyl}-acetonitrile;
2-[3-Methyl-4-(8-thiophen-3-yl-imidazo[4,5-c]quinolin-1-yl)-phenyl]-ethylamine;
2-{4-[8-(1H-Indol-5-yl)-imidazo[4,5-c]quinolin-1-yl]-3-methyl-phenyl}-ethylamine;
(R)-2-Amino-3-[4-(8-thiophen-2-yl-imidazo[4,5-c]quinolin-1-yl)-phenyl]-propionamide;
(R)-2-Amino-3-[4-(8-benzo[b]thiophen-2-yl-imidazo[4,5-c]quinolin-1-yl)-phenyl]-propionamide;
[3,5-Dichloro-4-(8-thiophen-2-yl-imidazo[4,5-c]quinolin-1-yl)-phenyl]-acetonitrile;
[3,5-Dichloro-4-(8-thiophen-3-yl-imidazo[4,5-c]quinolin-1-yl)-phenyl]-acetonitrile;
2-[3,5-Dichloro-4-(8-thiophen-2-yl-imidazo[4,5-c]quinolin-1-yl)-phenyl]-ethylamine;
2-[3,5-Dichloro-4-(8-thiophen-3-yl-imidazo[4,5-c]quinolin-1-yl)-phenyl]-ethylamine;
{4-[8-(4-Hydroxy-phenyl)-imidazo[4,5-c]quinolin-1-yl]-3-trifluoromethyl-phenyl}-acetonitrile;
4-{1-[4-(2-Amino-ethyl)-2-trifluoromethyl-phenyl]-1H-imidazo[4,5-c]quinolin-8-yl}-phenol;
3-[4-(8-Benzo[1,3]dioxol-5-yl-imidazo[4,5-c]quinolin-1-yl)-phenyl]-propionitrile;
3-[4-(8-Thiophen-2-yl-imidazo[4,5-c]quinolin-1-yl)-phenyl]-propionitrile;
3-[4-(8-Benzo[b]thiophen-2-yl-imidazo[4,5-c]quinolin-1-yl)-phenyl]-propionitrile;
3-[4-(8-Thiophen-3-yl-imidazo[4,5-c]quinolin-1-yl)-phenyl]-propionitrile;
3-[4-(8-Benzofuran-2-yl-imidazo[4,5-c]quinolin-1-yl)-phenyl]-propionitrile;
3-[4-(8-Benzo[1,3]dioxol-5-yl-imidazo[4,5-c]quinolin-1-yl)-phenyl]-propylamine;
3-[4-(8-Thiophen-2-yl-imidazo[4,5-c]quinolin-1-yl)-phenyl]-propylamine;
3-[4-(8-Benzo[b]thiophen-2-yl-imidazo[4,5-c]quinolin-1-yl)-phenyl]-propylamine;
3-[4-(8-Thiophen-3-yl-imidazo[4,5-c]quinolin-1-yl)-phenyl]-propylamine;
3-[4-(8-Benzofuran-2-yl-imidazo[4,5-c]quinolin-1-yl)-phenyl]-propylamine;
3-[4-(8-Benzo[1,3]dioxol-5-yl-imidazo[4,5-c]quinolin-1-yl)-3-chloro-phenyl]-propionitrile;
3-[3-Chloro-4-(8-thiophen-2-yl-imidazo[4,5-c]quinolin-1-yl)-phenyl]-propionitrile;
3-[3-Chloro-4-(8-thiophen-3-yl-imidazo[4,5-c]quinolin-1-yl)-phenyl]-propionitrile;
3-{3-Chloro-4-[8-(1H-indol-5-yl)-imidazo[4,5-c]quinolin-1-yl]-phenyl}-propionitrile;
3-[4-(8-Benzo[1,3]dioxol-5-yl-imidazo[4,5-c]quinolin-1-yl)-3-chloro-phenyl]-propylamine;
3-[3-Chloro-4-(8-thiophen-2-yl-imidazo[4,5-c]quinolin-1-yl)-phenyl]-propylamine;
3-[3-Chloro-4-(8-thiophen-3-yl-imidazo[4,5-c]quinolin-1-yl)-phenyl]-propylamine;
3-{3-Chloro-4-[8-(1H-indol-5-yl)-imidazo[4,5-c]quinolin-1-yl]-phenyl}-propylamine;
8-Benzo[1,3]dioxol-5-yl-1-{4-[2-(4,5-dihydro-1H-imidazol-2-yl)-ethyl]-phenyl}-1H-imidazo[4,5-c]quinoline;
[3-Chloro-4-(2-methyl-8-thiophen-2-yl-imidazo[4,5-c]quinolin-1-yl)-phenyl]-acetonitrile;
[3-Chloro-4-(2-methyl-8-thiophen-3-yl-imidazo[4,5-c]quinolin-1-yl)-phenyl]-acetonitrile;
2-[3-Chloro-4-(2-methyl-8-thiophen-2-yl-imidazo[4,5-c]quinolin-1-yl)-phenyl]-ethylamine;
2-[3-Chloro-4-(2-methyl-8-thiophen-3-yl-imidazo[4,5-c]quinolin-1-yl)-phenyl]-ethylamine;
[4-(8-Benzo[1,3]dioxol-5-yl-7-fluoro-imidazo[4,5-c]quinolin-1-yl)-phenyl]-acetonitrile;
[4-(7-Fluoro-8-thiophen-2-yl-imidazo[4,5-c]quinolin-1-yl)-phenyl]-acetonitrile;
[4-(7-Fluoro-8-thiophen-3-yl-imidazo[4,5-c]quinolin-1-yl)-phenyl]-acetonitrile;
[4-(8-Benzofuran-2-yl-7-fluoro-imidazo[4,5-c]quinolin-1-yl)-phenyl]-acetonitrile;
{4-[7-Fluoro-8-(1H-indol-5-yl)-imidazo[4,5-c]quinolin-1-yl]-phenyl}-acetonitrile;
[4-(8-Benzo[b]thiophen-2-yl-7-fluoro-imidazo[4,5-c]quinolin-1-yl)-phenyl]-acetonitrile;
2-[4-(8-Benzo[1,3]dioxol-5-yl-7-fluoro-imidazo[4,5-c]quinolin-1-yl)-phenyl]-ethylamine;
2-[4-(7-Fluoro-8-thiophen-2-yl-imidazo[4,5-c]quinolin-1-yl)-phenyl]-ethylamine;
2-[4-(7-Fluoro-8-thiophen-3-yl-imidazo[4,5-c]quinolin-1-yl)-phenyl]-ethylamine;
2-[4-(8-Benzofuran-2-yl-7-fluoro-imidazo[4,5-c]quinolin-1-yl)-phenyl]-ethylamine;
2-{4-[7-Fluoro-8-(1H-indol-5-yl)-imidazo[4,5-c]quinolin-1-yl]-phenyl}-ethylamine;
2-[4-(8-Benzo[b]thiophen-2-yl-7-fluoro-imidazo[4,5-c]quinolin-1-yl)-phenyl]-ethylamine;
3-[4-(8-Benzo[1,3]dioxol-5-yl-7-fluoro-imidazo[4,5-c]quinolin-1-yl)-phenyl]-propionitrile;
3-[4-(7-Fluoro-8-thiophen-2-yl-imidazo[4,5-c]quinolin-1-yl)-phenyl]-propionitrile;
3-[4-(7-Fluoro-8-thiophen-3-yl-imidazo[4,5-c]quinolin-1-yl)-phenyl]-propionitrile;
3-[4-(8-Benzofuran-2-yl-7-fluoro-imidazo[4,5-c]quinolin-1-yl)-phenyl]-propionitrile;
3-[4-(8-Benzo[b]thiophen-2-yl-7-fluoro-imidazo[4,5-c]quinolin-1-yl)-phenyl]-propionitrile;
3-{4-[7-Fluoro-8-(1H-indol-5-yl)-imidazo[4,5-c]quinolin-1-yl]-phenyl}-propionitrile;
3-[4-(8-Benzo[1,3]dioxol-5-yl-7-fluoro-imidazo[4,5-c]quinolin-1-yl)-phenyl]-propylamine;
3-[4-(7-Fluoro-8-thiophen-2-yl-imidazo[4,5-c]quinolin-1-yl)-phenyl]-propylamine;
3-[4-(7-Fluoro-8-thiophen-3-yl-imidazo[4,5-c]quinolin-1-yl)-phenyl]-propylamine;
3-[4-(8-Benzofuran-2-yl-7-fluoro-imidazo[4,5-c]quinolin-1-yl)-phenyl]-propylamine;
3-[4-(8-Benzo[b]thiophen-2-yl-7-fluoro-imidazo[4,5-c]quinolin-1-yl)-phenyl]-propylamine;
3-{4-[7-Fluoro-8-(1H-indol-5-yl)-imidazo[4,5-c]quinolin-1-yl]-phenyl}-propylamine;
[3-Chloro-4-(7-fluoro-8-thiophen-2-yl-imidazo[4,5-c]quinolin-1-yl)-phenyl]-acetonitrile;
[3-Chloro-4-(7-fluoro-8-thiophen-3-yl-imidazo[4,5-c]quinolin-1-yl)-phenyl]-acetonitrile;
{3-Chloro-4-[7-fluoro-8-(1H-indol-5-yl)-imidazo[4,5-c]quinolin-1-yl]-phenyl}-acetonitrile;
2-[3-Chloro-4-(7-fluoro-8-thiophen-2-yl-imidazo[4,5-c]quinolin-1-yl)-phenyl]-ethylamine;
2-[3-Chloro-4-(7-fluoro-8-thiophen-3-yl-imidazo[4,5-c]quinolin-1-yl)-phenyl]-ethylamine;
2-{3-Chloro-4-[7-fluoro-8-(1H-indol-5-yl)-imidazo[4,5-c]quinolin-1-yl]-phenyl}-ethylamine;
[4-(7-Chloro-8-thiophen-2-yl-imidazo[4,5-c]quinolin-1-yl)-phenyl]-acetonitrile;
[4-(7-Chloro-8-thiophen-3-yl-imidazo[4,5-c]quinolin-1-yl)-phenyl]-acetonitrile;
[4-(8-Benzofuran-2-yl-7-chloro-imidazo[4,5-c]quinolin-1-yl)-phenyl]-acetonitrile;
{4-[7-Chloro-8-(1H-indol-5-yl)-imidazo[4,5-c]quinolin-1-yl]-phenyl}-acetonitrile;
[4-(8-Benzo[b]thiophen-2-yl-7-chloro-imidazo[4,5-c]quinolin-1-yl)-phenyl]-acetonitrile;
2-[4-(7-Chloro-8-thiophen-2-yl-imidazo[4,5-c]quinolin-1-yl)-phenyl]-ethylamine;
2-[4-(7-Chloro-8-thiophen-3-yl-imidazo[4,5-c]quinolin-1-yl)-phenyl]-ethylamine;
2-[4-(8-Benzofuran-2-yl-7-chloro-imidazo[4,5-c]quinolin-1-yl)-phenyl]-ethylamine;
2-{4-[7-Chloro-8-(1H-indol-5-yl)-imidazo[4,5-c]quinolin-1-yl]-phenyl}-ethylamine;
2-[4-(8-Benzo[b]thiophen-2-yl-7-chloro-imidazo[4,5-c]quinolin-1-yl)-phenyl]-ethylamine;
3-[4-(8-Benzo[1,3]dioxol-5-yl-7-chloro-imidazo[4,5-c]quinolin-1-yl)-phenyl]-propionitrile;
3-[4-(7-Chloro-8-thiophen-2-yl-imidazo[4,5-c]quinolin-1-yl)-phenyl]-propionitrile;
3-[4-(8-Benzofuran-2-yl-7-chloro-imidazo[4,5-c]quinolin-1-yl)-phenyl]-propionitrile;
3-{4-[7-Chloro-8-(1H-indol-5-yl)-imidazo[4,5-c]quinolin-1-yl]-phenyl}-propionitrile;
3-[4-(8-Benzo[b]thiophen-2-yl-7-chloro-imidazo[4,5-c]quinolin-1-yl)-phenyl]-propionitrile;
3-[4-(8-Benzo[1,3]dioxol-5-yl-7-chloro-imidazo[4,5-c]quinolin-1-yl)-phenyl]-propylamine;
3-[4-(7-Chloro-8-thiophen-2-yl-imidazo[4,5-c]quinolin-1-yl)-phenyl]-propylamine;
3-[4-(8-Benzofuran-2-yl-7-chloro-imidazo[4,5-c]quinolin-1-yl)-phenyl]-propylamine;
3-{4-[7-Chloro-8-(1H-indol-5-yl)-imidazo[4,5-c]quinolin-1-yl]-phenyl}-propylamine;
3-[4-(8-Benzo[b]thiophen-2-yl-7-chloro-imidazo[4,5-c]quinolin-1-yl)-phenyl]-propylamine;
[3-Chloro-4-(7-chloro-8-thiophen-2-yl-imidazo[4,5-c]quinolin-1-yl)-phenyl]-acetonitrile;
[3-Chloro-4-(7-chloro-8-thiophen-3-yl-imidazo[4,5-c]quinolin-1-yl)-phenyl]-acetonitrile;
2-[3-Chloro-4-(7-chloro-8-thiophen-2-yl-imidazo[4,5-c]quinolin-1-yl)-phenyl]-ethylamine;
2-[3-Chloro-4-(7-chloro-8-thiophen-3-yl-imidazo[4,5-c]quinolin-1-yl)-phenyl]-ethylamine;
3-[3-Chloro-4-(7-chloro-8-thiophen-2-yl-imidazo[4,5-c]quinolin-1-yl)-phenyl]-propionitrile;
3-[3-Chloro-4-(7-chloro-8-thiophen-3-yl-imidazo[4,5-c]quinolin-1-yl)-phenyl]-propionitrile;
3-{3-Chloro-4-[7-chloro-8-(1H-indol-5-yl)-imidazo[4,5-c]quinolin-1-yl]-phenyl}-propionitrile;
3-[3-Chloro-4-(7-chloro-8-thiophen-2-yl-imidazo[4,5-c]quinolin-1-yl)-phenyl]-propylamine;
3-[3-Chloro-4-(7-chloro-8-thiophen-3-yl-imidazo[4,5-c]quinolin-1-yl)-phenyl]-propylamine;
3-{3-Chloro-4-[7-chloro-8-(1H-indol-5-yl)-imidazo[4,5-c]quinolin-1-yl]-phenyl}-propylamine;
3-[4-(2-Amino-7-chloro-8-thiophen-3-yl-imidazo[4,5-c]quinolin-1-yl)-phenyl]-propionitrile;
3-[4-(2-Amino-8-bromo-7-chloro-imidazo[4,5-c]quinolin-1-yl)-phenyl]-propionitrile;
3-[4-(3-Amino-6-bromo-7-chloro-quinolin-4-ylamino)-phenyl]-propionitrile;
3-[4-(2-Amino-8-benzofuran-2-yl-7-chloro-imidazo[4,5-c]quinolin-1-yl)-phenyl]-propionitrile;
1-[4-(3-Amino-propyl)-phenyl]-7-chloro-8-thiophen-3-yl-1H-imidazo[4,5-c]quinolin-2-ylamine;
1-[4-(3-Amino-propyl)-phenyl]-8-benzofuran-2-yl-7-chloro-1H-imidazo[4,5-c]quinolin-2-ylamine;
8-(2,4-Dimethoxy-phenyl)-1-(2-fluoro-phenyl)-1H-imidazo[4,5-c]quinoline;
8-(2,5-Dimethoxy-phenyl)-1-(2-fluoro-phenyl)-1H-imidazo[4,5-c]quinoline;
8-(3,4-Dimethoxy-phenyl)-1-(2-fluoro-phenyl)-1H-imidazo[4,5-c]quinoline;
1-(2-Fluoro-phenyl)-8-(3,4,5-trimethoxy-phenyl)-1H-imidazo[4,5-c]quinoline;
8-(2,3-Dimethoxy-phenyl)-1-(2-fluoro-phenyl)-1H-imidazo[4,5-c]quinoline;
1-(2-Fluoro-phenyl)-8-(2,3,4-trimethoxy-phenyl)-1H-imidazo[4,5-c]quinoline;
1-(2-Fluoro-phenyl)-8-pyridin-4-yl-1H-imidazo[4,5-c]quinoline;
1-(2-Fluoro-phenyl)-8-pyridin-3-yl-1H-imidazo[4,5-c]quinoline;
1-(2-Fluoro-phenyl)-8-(3-methoxy-phenyl)-1H-imidazo[4,5-c]quinoline;
{3-[1-(2-Fluoro-phenyl)-1H-imidazo[4,5-c]quinolin-8-yl]-benzyl}-dimethyl-amine;
1-(2-Fluoro-phenyl)-8-[3-(4-methyl-piperazin-1-ylmethyl)-phenyl]-1H-imidazo[4,5-c]quinoline;
1-(2-Fluoro-phenyl)-8-(3-morpholin-4-ylmethyl-phenyl)-1H-imidazo[4,5-c]quinoline;
1-(2-Fluoro-phenyl)-8-(3-piperazin-1-ylmethyl-phenyl)-1H-imidazo[4,5-c]quinoline;
1-(2-Fluoro-phenyl)-8-(3-pyrrolidin-1-ylmethyl-phenyl)-1H-imidazo[4,5-c]quinoline;
1-Phenyl-8-(3-piperazin-1-yl-phenyl)-1H-imidazo[4,5-c]quinoline;
8-(3-Fluoro-phenyl)-1-phenyl-1H-imidazo[4,5-c]quinoline;
8-[3-(4-Methyl-piperazin-1-yl)-phenyl]-1-phenyl-1H-imidazo[4,5-c]quinoline;
4-{1-[4-(4-Methyl-piperazin-1-yl)-phenyl]-1H-imidazo[4,5-c]quinolin-8-yl}-phenol;
1-[4-(4-Methyl-piperazin-1-yl)-phenyl]-8-phenyl-1H-imidazo[4,5-c]quinoline; and
[4-(8-Pyridin-4-yl-imidazo[4,5-c]quinolin-1-yl)-phenyl]-acetonitrile.

16. The method according to claim 10, wherein the protein kinase dependent disease is preferably one depending on PKB, ALK, S6K1 or RET and (especially aberrantly highly-expressed or activated) PKB, ALK, S6K1 or RET-dependent disease or disease dependent on the activation of the PKB, ALK, S6K1 or RET pathways, or a disease dependent on any combination thereof.
